(19) United States
(12) Patent Application Publication
(10) Pub. No.: US 2017/0058314 A1
(43) Pub. Date: Mar. 2, 2017

(54) NOVEL METHODOLOGY FOR IDENTIFYING ANTI-PERSISTER ACTIVITY AND ANTIMICROBIAL SUSCEPTIBILITY FOR BORRELIA BURGDORFERI AND OTHER BACTERIA

(71) Applicants: YING ZHANG, ELLICOTT CITY, MD (US); JIE FENG, BALTIMORE, MD (US); REBECCA YEE, BALTIMORE, MD (US); SHUO ZHANG, BALTIMORE, MD (US); WENHONG ZHANG, SHANGHAI (CN)

(72) Inventors: YING ZHANG, ELLICOTT CITY, MD (US); JIE FENG, BALTIMORE, MD (US); REBECCA YEE, BALTIMORE, MD (US); SHUO ZHANG, BALTIMORE, MD (US); WENHONG ZHANG, SHANGHAI (CN)

(21) Appl. No.: 15/347,285

(22) Filed: Nov. 9, 2016

Related U.S. Application Data
(63) Continuation-in-part of application No. PCT/US15/24122, filed on Apr. 2, 2015.
(60) Provisional application No. 62/073,605, filed on Oct. 31, 2014.
(60) Provisional application No. 62/136,678, filed on Mar. 23, 2015.

(51) Int. Cl. C12Q 1/18; C12Q 1/04

(52) U.S. Cl. CPC C12Q 1/18; C12Q 1/04

(57) ABSTRACT

The presently disclosed subject matter provides methods, compositions, and kits for assessing the viability of bacteria from a selected genus, assessing the antibiotic susceptibility of bacteria from the selected genus, and identifying compounds with anti-persister activity for bacteria from the selected genus. The bacteria include, but are not limited to, Borrelia burgdorferi, Staphylococcus aureus, Escherichia coli, Klebsiella pneumoniae, Acinetobacter baumanii, and Mycobacterium tuberculosis. Compositions include compounds with high activity against Borrelia persisters and their combinations with current Lyme antibiotics for more effect treatment of Lyme disease. Methods for inhibiting the growth and/or survival of bacteria from the Borrelia genus and for treating Lyme disease using appropriate drug combinations in a subject are also provided.

### CROSS-REFERENCE TO RELATED APPLICATIONS

This application is a Continuation-in-Part of international application PCT/US2015/24122, having an international filing date of Apr. 2, 2015, which claims the benefit of U.S. Provisional Application No. 62/073,605, filed Oct. 31, 2014, and U.S. Provisional Application No. 62/136,678, filed Mar. 23, 2015, each of which are incorporated herein by reference in their entirety.

### FEDERALLY SPONSORED RESEARCH OR DEVELOPMENT

This invention was made with government support under A1099512 and AI108535 awarded by the National Institutes of Health (NIH). The government has certain rights in the invention.

### BACKGROUND

Lyme disease is a multisystem disease caused by the spirochetal bacterium Borrelia burgdorferi (Meek et al., 1996; Stricker et al., 2011). The disease is transmitted by tick vectors that can be spread by rodents, reptiles, birds and deer (Stricker et al., 2011; Radolf et al., 2012). In the United States, the number of Lyme disease cases has doubled in the last 15 years (Orloski et al., 2000; Bacon et al., 2008) and is estimated to be about 300,000 cases each year (Centers for Disease Control, 2014). Currently, Lyme disease is considered the most common tick-borne disease in the United States and Europe (Bacon et al., 2008; Armed Forces Health Surveillance; 2001-2012; CDC, Lyme Disease, 2014). The clinical manifestations of early Lyme disease are most often characterized by an erythema migrans rash often accompanied by flu-like symptoms. An inflammatory arthritis or neurological dysfunction can be frequent sequelae of untreated infection.

The majority of patients with Lyme disease can be cured with the antibiotics doxycycline or amoxicillin used for a duration of 2-4 weeks, especially during the early phase of the disease. However, a subset of patients experience persistent symptoms despite antimicrobial therapy including fatigue, neurocognitive difficulties (“brain fog”) or musculoskeletal pains. When symptoms last longer than 6 months after antibiotic treatment, this has been proposed as a non-infectious, post-treatment Lyme disease syndrome (PTLDS) due to the inability to find viable, remaining organisms and lack of substantial efficacy with longer term monotherapy with ceftriaxone, doxycycline or amoxicillin (CDC, Post-Treatment Lyme Disease Syndrome, 2014; Wormser et al., 2006; Klempner et al., 2013). About 10-20% of patients suffer from PTLDS or chronic persistent Lyme disease (Centers for Disease Control, 2014), especially those patients who missed early diagnosis and treatment because of unnoticed tick-bites or inaccurate testing (Stricker et al., 2011).

It is unclear what mechanisms promulgate this condition in these patients. Concepts raised have included host responses, although slow or ineffective killing of B. burgdorferi persisters has been voiced as a possible explanation, even though evidence of viable organisms present in PTLDS is lacking (Bemdtson, 2013). Previous studies showed B. burgdorferi might still persist in a number of patients after a short course of antibiotics (Stricker et al., 2011; Hodzic et al., 2008; Diterich et al., 2003).

While PTLDS has only subjective symptom complexes, about 10% of patients with late Lyme arthritis have objective, persistent joint swelling despite antibiotic therapy (antibiotic refractory Lyme arthritis; Steere and Glickstein, 2004). Though part of this response may include autoimmune mimicry induced by B. burgdorferi in certain hosts, an additional explanation rests on immunological responses driven by continued infection or presence of antigenic debris (Bockenstedt et al., 2012).

The question of whether B. burgdorferi might still persist in some patients after antibiotic therapy and further evade host immune clearance has been raised by some, but is controversial (Stricker et al., 2011; Hodzic et al., 2008; Diterich et al., 2003). In various animal models, such as mice, dogs and monkeys, antibiotic therapy with doxycycline, ceftriaxone or tigecycline could not fully eradicate detection of B. burgdorferi as shown by xenodiagnoses and PCR even though viable organisms could not be cultured in conventional culture medium (Barthold et al., 2010; Embers et al., 2012; Hodzic et al., 2014; Straubinger et al., 1997). Recently demonstrated, post-antibiotic persistence was present with resurgence of non-culturable B. burgdorferi DNA found in mice 12 months after antibiotic treatment (Hodzic et al., 2014). A human study with tick xenodiagnosis showed some patients after treatment still had Borrelia bacteria (Marques et al., 2014). These observations suggest some form of persistent B. burgdorferi that antibiotic dosings employed are not able to completely eradicate, though antibiotic levels in the animal experiments may have been inadequate.

No effective antibiotic for treating chronic persistent Lyme disease is currently available. The currently used frontline drugs, such as doxycycline, amoxicillin, and minocycline, have limited activity on persistent B. burgdorferi. A number of prospective, randomized clinical studies have found neither significant beneficial effect of additional prolonged antibiotic therapy with conventionally employed antibiotic monotherapy nor evidence of continued presence of B. burgdorferi in patients with long-term symptoms (Klempner et al., 2013; Fallon et al., 2008). One study did report some improvement in fatigue symptoms with prolonged intravenous administration with ceftriaxone, though ultimately not thought to be worth the risks to administer for this benefit alone (Krupp et al., 2003). Ceftriaxone has recently been shown to be more active against B. burgdorferi persisters than doxycycline or amoxicillin (Feng, Wang, Shi, et al., 2014). Intriguingly, a recent study in humans demonstrated the recovery of B. burgdorferi DNA by xenodiagnosis in a single patient with PTLDS despite antibiotic treatment (Marques et al., 2014).

B. burgdorferi is capable of a complex life style in vitro characterized by multiple pleomorphic forms including spirochetal, spheroplast (or L-form), cyst or round body (RB), and microcolony forms (Diterich et al., 2003; Brorson et al., 2009; Sapi et al., 2012; Miklossy et al., 2008; Alban et al., 2000; Hodzic et al., 2008). These morphological variants of B. burgdorferi have different antibiotic susceptibilities (Sapi et al., 2011). RB forms appear as coccoid, membrane-bound atypical variants of B. burgdorferi, forming under experimental stress conditions, such as starvation, oxidative stress, pH variations, heating, or antibiotic treatment in culture (Brorson et al., 2009; Murgia and Cinco, 2004; Brorson and Brorson, 1997; Kersten et al., 1995). The RB forms, which have lower metabolism and resist diverse stresses, might be a protective mechanism to overcome adverse environmental conditions (Murgia and Cinco, 2004; Brorson et al., 2009). These are relatively refractory to killing by many antibiotics including doxycycline and amoxicillin (Feng, Wang, Shi, et al., 2014; Brorson et al., 2009), and can revert to classical helical spirochetal forms in fresh nonantibiotic-containing subculture (Brorson et al., 2009; Brorson and Brorson, 1998; Murgia and Cinco, 2004).

The round body forms of B. burgdorferi are also found in vivo during B. burgdorferi infection as seen in the spinal fluid (Brorson and Brorson, 1998) and the brain tissues of chronic Lyme neuroborreliosis patients (Miklossy et al., 2008). These findings suggest that the round body form of B. burgdorferi might play a role in chronic Lyme disease.

Although atypical cystic or granular forms have been described in humans (Miklossy et al., 2008), there is neither good evidence that such morphologic variants are common with human infection nor that additional antibiotics improves patients with persistent symptoms after initial treatment (Lantos et al., 2014). While frontline drugs doxycycline, amoxicillin, and minocycline kill replicating spirochetal form of B. burgdorferi quite effectively, they have little activity against non-replicating persisters or biofilm-like aggregates or microcolonies of B. burgdorferi enriched within stationary phase cultures (Feng, Wang, Shi, et al., 2014; Sapi et al., 2011).

In addition, although some antibiotics have been tested for their activity against B. burgdorferi, the full spectrum of antibiotic susceptibility in B. burgdorferi has not been determined (Hunfeld and Brade, 2006). Thus, searching for effective antibiotics and their combinations is important to develop effective therapy for chronic Lyme disease. The FDA-approved drugs already have relatively clear safety and pharmacokinetic profiles in patients, as well as manufacturing and distribution networks. Therefore, approved drugs could rapidly be applied in treatments for Lyme disease if they prove to have activity.

Screening for new antibiotics with activity against B. burgdorferi is difficult with the current viability assays, which are primarily based on microscopic counting and PCR. These assays are tedious and cannot be used for high-throughput screens. The commonly used LIVE/DEAD BacLight assay has a high background problem and cannot be used directly for viability assessment of bacteria, such as Borrelia burgdorferi, or other bacteria of interest, in a high-throughput format.

### SUMMARY

In one aspect, the presently disclosed subject matter provides methods for assessing the viability or assessing the antimicrobial susceptibility of spirochetal organisms, such as from the Borrelia genus, and more preferably, the Borrelia burgdorferi species, and related organisms, and other bacteria, including, but not limited to, Staphylococcus aureus, Escherichia coli, Klebsiella pneumoniae, Acinetobacter baumanii, and Mycobacterium tuberculosis.

Accordingly, in some aspects, the presently disclosed subject matter provides a method for assessing the viability of bacteria from a selected genus, the method comprising: (a) establishing a bacterial culture comprising isolated bacteria from the selected genus; (b) incubating the bacterial culture with a staining mixture comprising: (i) a first agent which emits fluorescence of a first color that is indicative of live bacterial cells in the culture, and (ii) a second agent which emits fluorescence of a second color that contrasts from the first color and is indicative of dead bacterial cells in the culture; and (c) calculating a ratio of the intensity of emitted fluorescence of the first color to the intensity of emitted fluorescence the second color; and (d) assessing the viability of the bacteria in the culture, wherein the ratio calculated in (c) is indicative of the percentage of live bacteria in the culture.

In certain aspects, the presently disclosed subject matter provides a method for assessing the susceptibility of bacteria from a selected genus to at least one antimicrobial agent, the method comprising: (a) establishing a bacterial culture comprising isolated bacteria from the selected genus; (b) incubating the culture under suitable conditions for bacterial growth to occur with: (i) at least one dose of at least one antimicrobial agent; and (ii) a staining mixture comprising a first agent which emits fluorescence of a first color that is indicative of live bacteria in the culture, and a second agent which emits fluorescence of a second color that contrasts from the first color and is indicative of dead bacteria in the culture, wherein a ratio of the intensity of emitted fluorescence of the first color to the intensity of emitted fluorescence of the second color is indicative of the percentage of live bacteria in the culture; and (c) assessing the susceptibility of bacteria from the selected genus to at least one antimicrobial agent by calculating the ratio in (b)(ii) without the need for bacterial growth, after a period of exposure to at least one dose of at least one antimicrobial agent, wherein the bacteria are assessed as susceptible to at least one antimicrobial agent if the ratio in (b)(ii) remains the same or decreases after the period of exposure to at least one dose of at least one antimicrobial agent, and wherein the bacteria are assessed as resistant to at least one antimicrobial agent if the ratio in (b)(ii) increases after the period of exposure to at least one dose of at least one agent.

In particular aspects, the method is performed in a high-throughput format, such as for drug screens.

In other aspects, the presently disclosed subject matter provides a method for identifying a candidate agent that is capable of inhibiting growth or survival of bacteria from a selected genus, the method comprising: (a) establishing a culture comprising isolated bacteria from the selected genus; (b) contacting the culture with a test agent; (c) assessing a viability of the bacteria in the culture in the presence of the test agent as compared to the viability of the bacteria in a control culture which lacks the test agent for drug screens on non-growing persisters in continuous time lapse manner over different time span and rapid antimicrobial susceptibility testing in a growth independent manner such that susceptibility to a test agent can be rapidly determined without relying on bacterial growth as in conventional antibiotic susceptibility testing, wherein assessing the viability of the bacteria in the culture comprises: (i) incubating the culture with a staining mixture comprising a first agent which emits fluorescence of a first color that is indicative of live bacteria, and a second agent which emits fluorescence of a second color that contrasts from the first color and is indicative of dead bacteria; (ii) calculating a ratio of the intensity of emitted fluorescence of the first color to the intensity of emitted fluorescence the second color, wherein the ratio is indicative of the percentage of live bacteria in the culture; and (d) identifying the test agent as a candidate agent that is capable of inhibiting growth or survival of bacteria from the selected genus if the calculated ratio for the culture is decreased relative to a similarly calculated ratio for the control culture.

In some aspects, the presently disclosed subject matter provides a kit for screening for at least one agent that is capable of inhibiting growth or survival of bacteria from a selected genus, the kit comprising isolated non-replicating persister forms of bacteria from the selected genus and reagents for performing a SYBR Green I/Propidium iodide viability assay.

In certain aspects, the presently disclosed subject matter provides a kit for assessing the viability and sensitivity of cultures of bacteria from a selected genus for at least one agent (e.g., current Lyme disease antibiotics or any new agents) that is capable of inhibiting growth or survival of bacteria from the selected genus, the kit comprising cultures of bacteria from the selected genus, reagents for performing a SYBR Green I/Propidium iodide assay, and optionally at least one test agent.

In other aspects, the presently disclosed subject matter provides a kit for screening at least one candidate agent that is capable of inhibiting growth or survival of bacteria from a selected genus, the kit comprising: (a) a population of isolated bacteria comprising bacteria from the selected genus or a culture thereof; (b) a staining mixture comprising: (i) a first agent which emits fluorescence of a first color that is indicative of live bacterial cells, and (ii) a second agent which emits fluorescence of a second color that contrasts from the first color and is indicative of dead bacterial cells, wherein when the staining mixture is incubated with the bacteria population or culture thereof a calculated ratio of the intensity of emitted fluorescence of the first color to the intensity of emitted fluorescence the second color is indicative of the percentage of live bacteria in the population or culture thereof; and (c) instructions for using the bacteria in (a) and the staining mixture in (b) to screen for at least one candidate agent that is capable of inhibiting growth or survival of bacteria from the selected genus.

In certain aspects of the presently disclosed methods, the selected genus is the Borrelia genus. In particular aspects, the bacteria are Borrelia burgdorferi. In other aspects, the selected genus is selected from the group consisting of Staphylococcus, Escherichia, Klebsiella, Acinetobacter, and Mycobacterium. In yet more particular aspects, the bacteria are selected from the group consisting of Staphylococcus aureus, Escherichia coli, Klebsiella pneumoniae, Acinetobacter baumanii, and Mycobacterium tuberculosis.

In certain aspects, the presently disclosed subject matter provides a method for inhibiting the growth and/or survival of bacteria from a selected genus, the method comprising contacting bacteria from the selected genus with an effective amount of: (a) at least one compound selected from the group consisting of daptomycin, artemisinin, ciprofloxacin, sulfacetamide, sulfamethoxypyridazine, nifuroxime, fosfomycin, chlortetracycline, sulfathiazole, clofazimine, cefmenoxime, cefoperazone, carbomycin, cefotiam, cefepime, amodiaquin, fosfomycin and streptomycin; (b) at least one compound selected from the group consisting of daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]aminol-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne)dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; (c) a combination of at least two compounds comprising: (i) a first compound selected from the group consisting of daptomycin, cefoperazone, miconazole and sulfamethoxypyridazine; and (ii) a second compound other than the first compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne)dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; or (d) a combination of at least three compounds comprising: (i) doxycycline as a first compound; (ii) a second compound selected from the group consisting of daptomycin or cefoperazone; and (iii) a third compound other than the second compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne)dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone.

In other aspects, the presently disclosed subject matter provides a method for treating Lyme disease in a subject in need thereof, the method comprising administering to a subject an effective amount of: (a) at least one compound selected from the group consisting of daptomycin, artemisinin, ciprofloxacin, sulfacetamide, sulfamethoxypyridazine, nifuroxime, fosfomycin, chlortetracycline, sulfathiazole, clofazimine, cefmenoxime, cefoperazone, carbomycin, cefotiam, cefepime, amodiaquin, fosfomycin and streptomycin; (b) at least one compound selected from the group consisting of daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne)dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; (c) a combination of at least two compounds comprising: (i) a first compound selected from the group consisting of daptomycin, cefoperazone, miconazole and sulfamethoxypyridazine; and (ii) a second compound other than the first compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; or (d) a combination of at least three compounds comprising: (i) doxycycline as a first compound; (ii) a second compound selected from the group consisting of daptomycin or cefoperazone; and (iii) a third compound other than the second compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone.

In some aspects, the presently disclosed subject matter provides a method for treating Lyme disease in a subject in need thereof, the method comprising: (a) administering to the subject an effective amount of a combination of at least two agents comprising: (i) at least one agent that inhibits growth and/or survival of replicating forms of bacteria from the Borrelia genus; and (ii) at least one agent that inhibits growth and/or survival of non-replicating persister forms of bacteria from the Borrelia genus.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows representative morphological changes of Borrelia cells following treatment with antibiotics;

FIG. 2 shows a comparison of methods for assaying B. burgdorferi viability in a 96-well plate;

FIG. 3 shows a linear relationship between the percentage of live B. burgdorferi and the green/red fluorescence ratio of a SYBR Green/propidium iodide (PI) assay (¹BSK-H medium was removed in the washing steps prior to the LIVE/DEAD assay);

FIG. 4 shows the B. burgdorferi B31 strain with commonly used viability assays MTT, XTT, fluoroscein diacetate assay (FDA), the commercially available LIVE/DEAD BacLight assay, and the presently disclosed SYBR Green I/PI assay;

FIG. 5A, FIG. 5B, FIG. 5C, and FIG. 5D show the B. burgdorferi B31 strain observed with: (FIG. 5A) the fluorescent microscopy LIVE/DEAD BacLight stain; (FIG. 5B) SYBR Green/PI stain; (FIG. 5C) FDA stain; and (FIG. 5D) the B. burgdorferi biofilm stained by SYBR Green/PI;

FIG. 6 shows a SYBR Green/PI assay showing correlation with direct microscope counting in an antibiotic exposure (¹Samples were stained with LIVE/DEAD BacLight kit);

FIG. 7 shows a representative drawing of the Yin-Yang model of bacterial persisters and latent infections where it is proposed to target both growing and non-growing bacterial populations for more effective treatment of difficult to cure or persistent bacterial infections and even cancer (Zhang, 2014);

FIG. 8A, FIG. 8B, and FIG. 8C show: (FIG. 8A) a growth curve of B. burgdorferi strain B31 in vitro; (FIG. 8B) representative images of log phase (3 day culture) and stationary phase (7 day culture) of the B. burgdorferi B31 strain observed with fluorescent microscopy using SYBR Green I/PI stain; the arrows indicate multiple morphological forms of B. burgdorferi in stationary phase; and (FIG. 8C) susceptibility of log phase (3 days) and stationary phase (7 days) B. burgdorferi to 50 M drugs after 5-day treatment. The percentages of residual live cells were determined by SYBR Green I/PI assay;

FIG. 9 shows the screening of a FDA-approved drug library (2,000 compounds) on stationary phase Borrelia persisters. In vitro activity of some effective antibiotics against stationary phase B. burgdorferi (cultured for 7 days) is shown;

FIG. 10A, FIG. 10B, FIG. 10C, and FIG. 10D show representative images of the stationary phase of B. burgdorferi strain B31 treated by: (FIG. 10A) daptomycin; (FIG. 10B) cefoperazone; (FIG. 10C) tetracycline; and (FIG. 10D) drug-free control. Treatment was followed by staining with SYBR Green I/PI;

FIG. 11 shows representative images of the stationary phase of B. burgdorferi strain B31 treated by carbomycin (left) and clofazimine (right);

FIG. 12 shows antibiotic minimum inhibitory concentrations (MICs) of some persister-active antibiotics for B. burgdorferi strain B31;

FIG. 13A, FIG. 13B, and FIG. 13C show representative images of: (FIG. 13A) 3-day-old log; (FIG. 13B) 7-day-old stationary; and (FIG. 13C) 10-day-old stationary phase B. burgdorferi cultures. The B. burgdorferi cultures of varying ages were stained with SYBR Green I/PI assay and observed under the microscope (400× magnification). The arrows indicate the spirochete (s), round body (r), and microcolony (m) forms of B. burgdorferi in stationary phase cultures;

FIG. 14 shows the effect of drugs (50 μg/mL) and combinations on stationary phase Borrelia. Susceptibility of stationary phase B. burgdorferi to drugs alone and their combinations after 5 days treatment. G/R: Green/Red ratio. Bracketed values: microscope counting percentages of residual viable cell. Dox: doxcycline; Amox: amoxillin; Cef-P: cefoperazone; Cef-T:ceftriaxone; MTZ: metronidazone; CFZ: clofazimine; MCZ: miconazole; PMB: polymyxin B;

FIG. 15 shows representative drug combinations against Borrelia biofilm. Images captured with epi-fluorescence inverted microscope (20× magnification). Drug concentration, 50 μg/mL;

FIG. 16 shows the activity of representative drug combinations against Borrelia biofilm. Fluorescence intensity and area of image were calculated by Image Pro Plus software;

FIG. 17A and FIG. 17B show the effect of antibiotics alone and in combinations on aggregated microcolony form and planktonic forms of B. burgdorferi. Stationary phase B. burgdorferi culture (10-day old) was treated with 10 μg/mL drugs (labeled on the image) for 7 days followed by staining by SYBR Green I/PI assay. Green cells indicate live cells whereas red cells indicate dead cells: (FIG. 17A) the B. burgdorferi aggregated microcolony (MC) form was more resistant to different antibiotics or their combinations than the planktonic form (round body and spirochetal form) (PT) as observed by fluorescence microscopy at 400× magnification; and (FIG. 17B) susceptibility of the B. burgdorferi microcolony form to antibiotics and antibiotic combinations was assessed by fluorescence microscopy at 200× magnification. The luminance of an individual RB is much weaker than that of a microcolony, which made the individual cells hard to observe when the microcolonies were being examined. Abbreviation: Dox, doxycycline; CefP, cefoperazone; Cfz, clofazimine; Dap, daptomycin; Smx, sulfamethoxazole; Cab, carbencillin; Car, carbomycin;

FIG. 18A, FIG. 18B, FIG. 18C, FIG. 18D, FIG. 18E, FIG. 18F, FIG. 18G, FIG. 18H, FIG. 18I show subculture (15 days) of 10-day-old B. burgdorferi stationary phase culture treated with different antibiotics alone or in combinations. Representative images were taken with fluorescence microscopy (400× magnification) using SYBR Green I/PI staining. Only Dox+Dap+CefP completely killed all forms including the microcolony form of B. burgdorferi persisters as shown by lack of any viable green spirochetal form after 15 day subculture. Abbreviation: Dox, doxycycline; CefP, cefoperazone; Cfz, clofazimine; Dap, daptomycin; Smx, sulfamethoxazole;

FIG. 19A, FIG. 19B, FIG. 19C, FIG. 19D show microscopy demonstrating round body formation in the presence of amoxicillin and subsequent reversion to spirochetal form of B. burgdorferi during subculture: (FIG. 19A) 5-day old B. burgdorferi culture consisting primarily of spirochetal form; (FIG. 19B) coccoid round body forms formed from B. burgdorferi spirochetes upon treatment with amoxicillin (50 μg/mL) for 3 days; (FIG. 19C) reversion of round body form of B. burgdorferi from (FIG. 19B) to spirochetal form after 5 day subculture in fresh BSK-medium; and (FIG. 19D) 7-day old stationary phase B. burgdorferi treated with 100 μg/mL amoxicillin for 3 days;

FIG. 20 shows exposure of 5-day old spirochetes and amoxicillin-induced round body form of B. burgdorferi (5 days) to 50 μM doxycycline, cefuroxime, and ceftriaxone for 5 days. The percentages of residual live cells were determined by SYBR Green I/PI assay followed by fluorescence microscopy counting;

FIG. 21A, FIG. 21B, FIG. 21C, FIG. 21D, FIG. 21E, FIG. 21F, FIG. 21G, FIG. 21H, FIG. 21I show representative images of amoxicillin-induced round body form of B. burgdorferi (6-day old culture induced with 50 μg/mL amoxicillin for 72 hours) treated with different antibiotics (50 μM) for 7 days followed by staining with SYBR Green I/PI assay and fluorescence microscopy;

FIG. 22A, FIG. 22B, FIG. 22C, FIG. 22D, FIG. 22E, FIG. 22F, FIG. 22G, FIG. 22H, FIG. 22I, FIG. 22J, FIG. 22K, FIG. 22L, FIG. 22M, FIG. 22N, FIG. 22O, FIG. 22P show the effect of antibiotics alone or in combinations on stationary phase B. burgdorferi microcolonies. Stationary phase culture of B. burgdorferi (10-day old) was treated with 10 μg/mL drugs alone or in combinations (labeled on the image) for 7 days followed by staining with SYBR Green I/PI assay. Green cells indicate live cells whereas red cells indicate dead cells. Abbreviation: Dox, doxycycline; CefP, cefoperazone; Art, Artemisinin; Dap, daptomycin; CefM, cefmetazole; Scp, sulfachlorpyridazine;

FIG. 23A, FIG. 23B, FIG. 23C, FIG. 23D, FIG. 23E, FIG. 23F, FIG. 23G, FIG. 23H, FIG. 23I show subculture (20 days) of the amoxicillin-induced round body form of B. burgdorferi (6-day old culture induced with 50 μg/mL amoxicillin for 72 hours) treated with different antibiotics alone or in combinations. Representative images were taken with fluorescence microscopy using SYBR Green I/PI staining. Only Dox+Dap+CefP completely killed all round body form of B. burgdorferi persisters as shown by lack of any viable green spirochetal form after 20-day subculture (FIG. 23G). Abbreviation: Dox, doxycycline; CefP, cefoperazone; Dap, daptomycin; Art: artemisinin; Scp, sulfachlorpyridazine;

FIG. 24 shows representative images of stationary phase B. burgdorferi treated with different compounds (50 μM) followed by staining with SYBR Green I/PI assay. Abbreviation: DOX: doxycycline, AMO: amoxicillin, DAP: daptomycin, DAU: daunomycin, NOG: nogalamycin, PYR: pyrromycin, RHO: Rhodomycin A, CHA: chaetochromin, PRO: prodigiosin, MIT: mitomycin, NAN: nanaomycin, DAC: dactinomycin, EMO: emodin;

FIG. 25 shows representative images of stationary phase B. burgdorferi strain B31 treated with different compounds (20 μM) followed by staining with SYBR Green I/PI assay. Abbreviation: DOX: doxycycline, DAP: daptomycin, DAU: daunomycin, NOG: nogalamycin, PYR: pyrromycin, RHO: Rhodomycin A, CHA: chaetochromin, PRO: prodigiosin, NAN: nanaomycin;

FIG. 26A, FIG. 26B, FIG. 26C, FIG. 26D, FIG. and 26E show a linear relationship between the percentage of live cells of FIG. 26A, Staphylococcus aureus (USA300), FIG. 26B, Escherichia coli (W3110), FIG. 26C, Klebsiella pneumoniae (Isolate 7), FIG. 26D Acinetobacter baumanii, and FIG. 26E, Mycobacterium tuberculosis (H37Ra), and the green/red fluorescence ratio from the SYBR GREEN/propidium iodide (PI) viability assay;

FIG. 27A, FIG. 27B, FIG. 27C, FIG. 27D, FIG. 27E, and FIG. 27F show the results of S. aureus USA300 strain (FIG. 27A, FIG. 27B, and FIG. 27C) and K. pneumoniae Isolate 7 strain (FIG. 27D, FIG. 27E, and FIG. 27F) observed with fluorescence microscopy after SYBR Green/PI staining of different viable proportions. Live cells are stained in green and dead cells treated with 70% isopropyl alcohol for 1 hr are stained in red;

FIG. 28A and FIG. 28B show the correlation of the SYBR Green/PI stain and traditional broth microdilution method for minimum inhibitory concentration (MIC) determination for (FIG. 28A) S. aureus USA300 strain and (FIG. 28B) E. coli W3110 strain. After following the AST protocol as described in CLSI, application of SYBR Green/PI staining to the bacteria cultures show that both SYBR Green/PI and OD600 reading indicate that the MIC of chloramphenicol against S. aureus USA300 is 4 μg/mL (FIG. 28A) and the MIC of ampicillin against E. coli W3110 is 8 μg/mL (FIG. 28B);

FIG. 29 shows the antimicrobial susceptibility testing results for S. aureus strains as determined by the traditional Kirby-Bauer Disk Diffusion method. Abbreviation: Gen, gentamicin; Kan, kanamycin; Tet, tetracycline; Ery, erythromycin; Rif, rifampicin; Cip, ciprofloxacin; Cm, chloramphenicol; --, did not test; S, sensitive; R, resistant; IR, intermediate resistance;

FIG. 30 shows that the SYBR Green/PI stain can distinguish between R (resistant) and S (sensitive) strains of S. aureus against various antibiotics in one and a half hours. Treatment of varying concentrations of bacteriostatic and bactericidal antibiotics (0 μg/mL, 5 μg/mL, 30 μg/mL, 50 μg/mL, and 100 μg/mL) were added to overnight S. aureus samples diluted to 1:500 in tryptic soy broth. After incubation with antibiotics for 1.5 hours, SYBR Green/PI staining was performed and distinguished the strains and their respective susceptibility categories. All susceptibility results were in concordance with results from the Kirby-Bauer disk diffusion test;

FIG. 31 shows the quantitative cut-off that can be used to distinguish susceptible and resistant strains of S. aureus. Using the green and red fluorescence values determined from the fluorescence microtiter plate reader, the proportion of killed cells was calculated with the formula (LD_{treated}−LD_{untreated})/LD_{untreated}, where LD is equal to the ratio of live (green fluorescence) and dead (red fluorescence) cells with treatment of 100 μg/mL of the respective drugs. The cut-offs distinguishing resistant and susceptible strains for the different antibiotics tested were established by averaging all the values (i.e. proportion of killed cells) from the respective susceptibility categories for all susceptible and resistant strains that were tested;

FIG. 32 shows that the SYBR Green/PI stain can distinguish between R (resistant) and S (sensitive) strains of S. aureus against various antibiotics in 30 minutes. Treatment of varying concentrations of bacteriostatic and bactericidal antibiotics (0 μg/mL to 400 μg/mL) were added to overnight S. aureus samples diluted to 1:25 in tryptic soy broth. After incubation with antibiotics for 30 minutes, SYBR Green/PI staining was performed and distinguished the strains and their respective susceptibility categories. All susceptibility results were in concordance with results from the Kirby-Bauer disk diffusion test;

FIG. 33 shows the antimicrobial susceptibility testing results for E. coli K-12 lab strain (W3110) and E. coli clinical strains as determined by the traditional Kirby-Bauer Disk Diffusion method. Abbreviation: Cm, chloramphenicol; Cip, ciprofloxacin; Trim, trimethoprim; Gen, gentamicin; Kan, kanamycin; Tet, tetracycline; Amp, ampicillin; Sm, streptomycin; S, sensitive; R, resistant; IR, intermediate resistance;

FIG. 34 shows that SYBR Green/PI stain can distinguish between R (resistant), IR (intermediate resistant), S (susceptible) strains of E. coli against various antibiotics in 1.5 hr. Treatment of varying concentrations of bacteriostatic and bactericidal antibiotics (0 μg/mL, 5 μg/mL, 30 μg/mL, 50 μg/mL, and 100 μg/mL) were added to overnight E. coli samples diluted to 1:500 in tryptic soy broth. After incubation with antibiotics for 1.5 hours, SYBR Green/PI staining was performed and distinguished the strains. All the susceptibility results were in concordance with results from the Kirby-Bauer disk diffusion test;

FIG. 35 shows the quantitative cut-off that can be used to distinguish susceptible and resistant strains of E. coli. Using the green and red fluorescence values determined from the fluorescence microtiter plate reader, the proportion of killed cells was calculated with the formula (LD_{treated}−LD_{untreated})/LD_{untreated}, where LD is equal to the ratio of Live (green fluorescence) and Dead (red fluorescence) cells. The cut-offs distinguishing resistant and susceptible strains for the different antibiotics at the designated concentrations were established by averaging all the values (i.e. proportion of killed cells) from the respective susceptibility categories for all susceptible and resistant strains that were tested. N.D=not determined;

FIG. 36 shows that SYBR Green/PI stain can distinguish between R (resistant) and S (susceptible) strains of E. coli against various antibiotics in 30 minutes. Treatment of varying concentrations of bacteriostatic and bactericidal antibiotics (0 μg/mL, 25 μg/mL, 50 μg/mL, 100 μg/mL, and 200 μg/mL) were added to overnight E. coli samples diluted to 1:25 in tryptic soy broth. After incubation with antibiotics for 30 minutes, SYBR Green/PI staining was performed and distinguished the strains. All the susceptibility results were in concordance with results from the Kirby-Bauer disk diffusion test;

FIG. 37 shows that SYBR Green/PI stain can distinguish between R (resistant) and S (susceptible) strains of K. pneumoniae against various antibiotics in 30 minutes. Treatment of varying concentrations of antibiotics (0 μg/mL, 25 μg/mL, 50 μg/mL, 100 μg/mL, and 200 μg/mL) were added to overnight E. coli W3110 and K. pneumoniae (Isolate 7) samples diluted to 1:25 in tryptic soy broth. After incubation with antibiotics for 30 minutes, SYBR Green/PI staining was performed and distinguished the strains. All the susceptibility results were in concordance with results from the Kirby-Bauer disk diffusion test;

FIG. 38 shows that the SYBR Green/PI stain can detect a similar trend in the number of E. coli persisters surviving antibiotic exposure compared to traditional colony forming unit (CFU) enumeration. The decrease in the level of persisters after exposure with the combination of polymycin B and gentamicin starting at Day 3 can be detected by both CFU enumeration and also green/red fluorescence ratio from SYBR Green/PI staining;

FIG. 39 shows linear relationship between M. tuberculosis H37Ra viability and Green/Red fluorescence ratio of the SYBR Green I/PI assay. Emission spectra of suspensions of various proportions of live and isopropyl alcohol-killed M. tuberculosis H37Ra were obtained, and the Green/Red fluorescence ratios were calculated for each proportion of live/dead cells. The line is a least-square fit of the relationship between percentage of live bacteria and Green/Red fluorescence ratio performed as described;

FIG. 40 shows representative images of M. tuberculosis H37Ra WT and PZA resistant mutants P5, P2 culture (20 day old), observed with fluorescence microscopy using SYBR Green I/PI stain. H37Ra WT and PZA resistant mutants were treated with 2 mg/mL PZA overnight (b, f, j). Salicylic acid (c, g, k) and acetic acid (d, h, 1) were used as enhancer to help increase the activity of PZA; and

FIG. 41 shows residual viability of M. tuberculosis H37Ra WT and PZA resistant mutants P5, P2 (20 day old) after overnight treatment with 2 mg/mL PZA. The residual viability was calculated according to the untreated control with Green/Red fluorescence ratios of SYBR Green I/PI assay. The final concentration of salicylic acid is 40 μg/mL and the final concentration of acetic acid is 8 μM. Compared with the H37Ra WT, the samples with statistically significant difference (P<0.05) compared with the control were marked with a star.

The patent or application file contains at least one drawing executed in color. Copies of this patent or patent application publication with color drawings will be provided by the Office upon request and payment of the necessary fee.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Methods for Identifying Anti-Persister Activity and Antimicrobial Susceptibility in Borrelia Burgdorferi and Other Bacteria

The presently disclosed subject matter relates to methods for assessing the viability of bacteria (e.g., from the Borrelia genus, e.g., replicating and/or non-replicating persister forms of B. burgdorferi and, in some embodiments, Staphylococcus aureus, Escherichia coli, Klebsiella pneumoniae, Acinetobacter baumanii, and Mycobacterium tuberculosis), methods for assessing the susceptibility of bacteria to candidate antimicrobial agents, methods for screening for at least one agent that inhibits the growth or survival of bacteria, methods for inhibiting the growth and/or survival of bacteria, methods of treating Lyme disease, and related compositions and kits that can be used to perform the methods.

Accordingly, in some embodiments, the presently disclosed subject matter provides a method for assessing the viability of bacteria from a selected genus, the method comprising: (a) establishing a bacterial culture comprising isolated bacteria from the selected genus; (b) incubating the bacterial culture with a staining mixture comprising: (i) a first agent which emits fluorescence of a first color that is indicative of live bacterial cells in the culture, and (ii) a second agent which emits fluorescence of a second color that contrasts from the first color and is indicative of dead bacterial cells in the culture; and (c) calculating a ratio of the intensity of emitted fluorescence of the first color to the intensity of emitted fluorescence the second color; and (d) assessing the viability of the bacteria in the culture, wherein the ratio calculated in (c) is indicative of the percentage of live bacteria in the culture.

In some embodiments, the presently disclosed subject matter provides a new SYBR Green I/propidium iodide (PI) (also termed SYBR Green/PI) assay based on a green fluorescence to red fluorescence ratio for rapid viability assessment of bacteria, such as those from the Borrelia genus or a genus selected from the group consisting of Staphylococcus, Escherichia, Klebsiella, Acinetobacter, and Mycobacterium. This assay is superior to other assays commonly used for measuring the viability and for rapid drug susceptibility testing of B. burgdorferi, and other bacteria, such as the current commercially available LIVE/DEAD BacLight viability assay (Invitrogen, Carlsbad, Calif.). The term “viability assay” as used herein refers to an assay to determine the ability of cells to maintain or recover viability, such as the ability to grow.

In other embodiments, the presently disclosed subject matter provides a method for assessing the viability of bacteria from a selected genus, the method comprising: (a) obtaining a culture comprising bacterial cells from the selected genus; (b) performing a viability assay of the bacterial cells in the culture by using a SYBR Green I/Propidium Iodide assay based on a ratio of green fluorescence, indicative of live bacterial cells, to red fluorescence, indicative of dead bacterial cells, comprising: (i) mixing the culture with a staining mixture comprising SYBR Green I and propidium iodide; (ii) allowing the culture and staining mixture to incubate in the dark; (iii) determining the fluorescence intensity of the culture and staining mixture at 535 nm, which measures green fluorescence, and 635 nm, which measures red fluorescence; and (iv) calculating the ratio of green fluorescence to red fluorescence; and wherein a ratio of green fluorescence to red fluorescence of more than approximately 7 means that the bacterial cells are viable.

In some embodiments, the first color is green and the second color is red or orange. In other embodiments, the first agent is SYBR Green I and the second agent is propidium iodide. In further embodiments, the SYBR Green I is present in the culture in a concentration range of between about 0.1× and about 100× and propidium iodide is present in the culture in a range of between about 0.1 mM and about 5 mM. In still further embodiments, the concentration of SYBR Green I in the culture is 10× and the concentration of propidium iodide is 2 mM.

In some embodiments, the culture further comprises a BSK-H medium. In other embodiments, the step of incubating the culture with the mixture is performed for approximately 15 minutes. In further embodiments, the step of incubating is performed in the dark.

Borrelia is a genus of bacteria of the Spirochete phylum. The Borrelia burgdorferi sensu lato complex includes at least 18 genospecies. Non-limiting examples of bacteria in this genus include B. burgdorferi, B. garinii, B. afzelii, B. americana, B. carolinensis, B. lusitaniae, B. japonica, B. miyamotoi and B. sinica. In some embodiments, the bacteria are Borrelia burgdorferi. In other embodiments, the Borrelia burgdorferi comprise a morphological form selected from the group consisting of a spirochete form, a spheroplast form, a cystic or round body form, a microcolony form, a biofilm-like and biofilm form, and combinations thereof.

In some embodiments, the method is performed in a high-throughput format, e.g., for drug screening. Unexpectedly, it has been found that the presently disclosed methods, in some embodiments, can be used without a washing step and for high-throughput screens. That is, the methods can be used to assess viability, susceptibility of bacteria to antimicrobials, and in drug screening directly in a high-throughput manner in the absence of washing. In other embodiments, the high-throughput format uses at least one multi-well microplate. Non-limiting examples of suitable multi-well microplates include, without limitation, a 6-well microplate, a 24-well microplate, a 96-well microplate, a 384-well microplate, and a 1536-well microplate. In still other embodiments, the multi-well microplate comprises a 96-well microplate.

In some embodiments, the presently disclosed subject matter provides a method for assessing the susceptibility of bacteria from a selected genus to at least one antimicrobial agent, the method comprising: (a) establishing a bacterial culture comprising isolated bacteria from the selected genus; (b) incubating the culture under suitable conditions for bacterial growth to occur with: (i) at least one dose of at least one antimicrobial agent; and (ii) a staining mixture comprising a first agent which emits fluorescence of a first color that is indicative of live bacteria in the culture, and a second agent which emits fluorescence of a second color that contrasts from the first color and is indicative of dead bacteria in the culture, wherein a ratio of the intensity of emitted fluorescence of the first color to the intensity of emitted fluorescence of the second color is indicative of the percentage of live bacteria in the culture; and (c) assessing the susceptibility of bacteria from the selected genus to at least one antimicrobial agent by calculating the ratio in (b)(ii) without the need for bacterial growth, after a period of exposure to at least one dose of at least one antimicrobial agent, wherein the bacteria are assessed as susceptible to at least one antimicrobial agent if the ratio in (b)(ii) remains the same or decreases after the period of exposure to at least one dose of at least one antimicrobial agent, and wherein the bacteria are assessed as resistant to at least one antimicrobial agent if the ratio in (b)(ii) increases after the period of exposure to at least one dose of at least one agent.

In other embodiments, the presently disclosed subject matter provides a method for screening for a compound that is capable of inhibiting bacteria from a selected genus, the method comprising: (a) obtaining a stationary phase bacterial culture that comprises bacterial cells from the selected genus; (b) contacting the culture with a test compound; (c) performing a viability assay of the bacterial cells in the culture by using a SYBR Green I/Propidium Iodide assay based on a ratio of green fluorescence, indicative of live bacterial cells, to red fluorescence, indicative of dead bacterial cells, comprising: (i) mixing the culture with a staining mixture comprising SYBR Green I and propidium iodide; (ii) allowing the culture and staining mixture to incubate in the dark; (iii) determining the fluorescence intensity of the culture and staining mixture at 535 nm, which measures green fluorescence, and 635 nm, which measures red fluorescence; (iv) calculating the ratio of green fluorescence to red fluorescence; and (v) comparing the ratio of green fluorescence to red fluorescence to the ratio of green fluorescence to red fluorescence of a set of controls with known amounts of live and dead bacterial cells to determine the percentage of live bacterial cells and dead bacterial cells in the culture; and (d) comparing the percentage of the live bacterial cells in the culture treated with the test compound to a control under identical conditions, but in the absence of the test compound; wherein a significant decrease in the percentage of the live bacterial cells and/or a significant increase in the percentage of dead bacterial cells in the culture with the test compound compared to the percentage of the live bacterial cells or dead bacterial cells in the control under identical conditions, but in the absence of the test compound is indicative that the test compound is capable of inhibiting the bacterial cells.

In some embodiments, the method further comprises determining a minimum inhibitory concentration breakpoint for at least one antimicrobial agent. In other embodiments, the first color is green and the second color is red or orange. In still other embodiments, the first agent is SYBR Green I and the second agent is propidium iodide. In still other embodiments, the concentration of SYBR Green I in the culture is about 10× and the concentration of propidium iodide is about 2 mM. In further embodiments, the culture further comprises a BSK-H medium.

In some embodiments, the bacteria are Borrelia burgdorferi. In other embodiments, the Borrelia burgdorferi comprise a morphological form selected from the group consisting of a spirochete form, a spheroplast form, a cystic or round body form, a microcolony form, a biofilm-like and biofilm form, and combinations thereof.

In some embodiments, the method is performed in a high-throughput format. In other embodiments, the high-throughput format uses at least one multi-well microplate. In further embodiments, the multi-well microplate comprises a 96-well microplate.

In some embodiments, the presently disclosed subject matter provides a method for identifying a candidate agent that is capable of inhibiting growth or survival of bacteria from a selected genus, the method comprising: (a) establishing a culture comprising isolated bacteria from the selected genus; (b) contacting the culture with a test agent; (c) assessing a viability of the bacteria in the culture in the presence of the test agent as compared to the viability of the bacteria in a control culture which lacks the test agent for drug screens on non-growing persisters in continuous time lapse manner over different time span and rapid antimicrobial susceptibility testing in a growth independent manner such that susceptibility to a test agent can be rapidly determined without relying on bacterial growth as in conventional antibiotic susceptibility testing, wherein assessing the viability of the bacteria in the culture comprises: (i) incubating the culture with a staining mixture comprising a first agent which emits fluorescence of a first color that is indicative of live bacteria, and a second agent which emits fluorescence of a second color that contrasts from the first color and is indicative of dead bacteria; (ii) calculating a ratio of the intensity of emitted fluorescence of the first color to the intensity of emitted fluorescence the second color, wherein the ratio is indicative of the percentage of live bacteria in the culture; and (d) identifying the test agent as a candidate agent that is capable of inhibiting growth or survival of bacteria from the selected genus if the calculated ratio for the culture is decreased relative to a similarly calculated ratio for the control culture. As used herein, a “similarly calculated ratio” means that the ratio for the control culture is calculated in the same manner as the ratio calculated for the bacterial culture.

In some embodiments, the bacteria are Borrelia burgdorferi. In other embodiments, the culture comprises a stationary phase culture. In still other embodiments, the stationary phase culture comprises non-replicating persister cells. In further embodiments, the stationary phase culture comprises morphological forms selected from the group consisting of round bodies, planktonic, and biofilm.

In some embodiments, the first color is green and the second color is red or orange. In other embodiments, the first agent is SYBR Green I and the second agent is propidium iodide. In still other embodiments, the concentration of SYBR Green I in the culture is about 10× and the concentration of propidium iodide is about 2 mM. In further embodiments, the culture further comprises a BSK-H medium.

In some embodiments, the method is performed in a high-throughput format. In other embodiments, the high-throughput format uses at least one multi-well microplate. In still other embodiments, the multi-well microplate comprises a 96-well microplate.

In some embodiments, the method includes conducting a microscopic counting rescreen to confirm that at least one test agent is a candidate agent for inhibiting growth or survival of bacteria.

As used herein, the terms “inhibit”, “inhibits”, or “significant decrease” means to decrease, suppress, attenuate, diminish, or arrest, for example the growth and/or survival of bacteria in a culture or in a subject, by at least 10%, 20%, 30%, 40%, 50%, 60%, 70%, 80%, 90%, 95%, 98%, 99%, or even 100% compared to an untreated control culture or subject. Inhibiting “survival” of bacteria in this context refers to killing bacteria or reducing live bacterial cell count. In some embodiments, the growth of the bacteria is inhibited by more than approximately 50%. In other embodiments, the percentage of live bacterial cells in the culture after the treatment with the test compound is less than approximately 50% compared to the percentage of live bacterial cells in the control under identical conditions, but in the absence of the test compound. In still other embodiments, the stationary phase bacterial culture comprises non-replicating persister cells. Further, as used herein, the term “significant increase” means an increase by at least 10%, 20%, 30%, 40%, 50%, 60%, 70%, 80%, 90%, 95%, 98%, 99%, or even 100%.

The test compound (used synonymously with agent herein) can be any compound or drug that is desirable to test for inhibitory activity, especially for anti-persister activity. The term “anti-persister activity” means that the compound has inhibitory activity against those bacterial cells that might still persist in a culture or subject after contact or administration with a course of antibiotics. The persistent bacteria may evade host immune clearance and result in chronic persistent infection in a subject. The test compound may be a known compound, such as an identified drug found to be effective in at least one disease or disorder, or may be an unknown compound that is not known to be effective in any disease or disorder. In some embodiments, the test compound is a known compound that has been approved by a health regulatory agency (e.g., FDA or EMA) for an indication other than treating chronic persistent Lyme disease. In some embodiments, the test compound is a compound that is known to exhibit antibiotic activity against bacteria other than those from the Borellia genus. In some embodiments, the test compound is a known compound that has not previously been reported to exhibit antibiotic activity against non-replicating persister forms of bacteria.

In some embodiments, the method is performed in a high-throughput format. By “high-throughput” format, it is meant that many samples, such as test compounds, can be tested at one time. For example, in other embodiments, the high-throughput format uses at least one 96-well plate. Of course, the ordinarily skilled artisan will appreciate that larger or smaller microplates can be used in a high-throughput format to carry out a method of the present disclosure.

As used herein, the term “bacterial culture” or “culture” refers to bacteria growing in a medium conducive for growth of those bacteria. The bacterial culture can be found in any type of container, such as a flask, a tube, a microwell plate, and the like. Generally, bacteria have different phases of growth. When a population of bacteria first enters a high-nutrient environment that allows growth, the cells need to adapt to their new environment. The first phase of growth is the lag phase, a period of slow growth when the cells are adapting to the high-nutrient environment and preparing for fast growth. The lag phase has high biosynthesis rates, as proteins necessary for rapid growth are produced. The second phase of growth is the log phase, also known as the logarithmic or exponential phase, in which the bacteria undergo rapid exponential growth. During log phase, nutrients are metabolized at maximum speed until one of the nutrients is depleted and starts limiting growth. The third phase of growth is the stationary phase and is caused by depleted nutrients. In some embodiments, a bacterial culture in “stationary phase” means that the bacteria in the culture have an approximately equal growth rate and death rate. As used herein, the term “growing forms” of bacteria generally refers to bacteria that are in lag or log phase and not in stationary phase. In some embodiments, the stationary phase bacterial culture has been grown for approximately 7 days. In other embodiments, the stationary phase bacterial culture comprises non-replicating persister cells. By “non-replicating persister cells,” it is meant bacterial cells that enter a state in which they stop replicating and are able to tolerate antibiotics.

### II. Kits for Identifying Anti-Persister Activity and Drug Susceptibility Evaluation

The presently disclosed subject matter also relates to kits for practicing the methods of the presently disclosed subject matter. In general, a presently disclosed kit contains some or all of the components, reagents, supplies, and the like to practice a method according to the presently disclosed subject matter. In some embodiments, the term “kit” refers to any intended any article of manufacture (e.g., a package or a container) comprising bacteria from the selected genus and an effective amount of reagents for performing a presently disclosed assay. The kit may also include a set of particular instructions for practicing the methods of the presently disclosed subject matter. In other embodiments, the presently disclosed subject matter provides a kit for screening for a compound that is capable of inhibiting bacteria from the selected genus, the kit comprising bacterial cells from the selected genus and reagents for performing a presently disclosed assay.

Accordingly, in some embodiments, the presently disclosed subject matter provides a kit for screening for at least one agent that is capable of inhibiting growth or survival of bacteria from a selected genus, the kit comprising isolated non-replicating persister forms of bacteria from the selected genus and reagents for performing a SYBR Green I/Propidium iodide viability assay.

In other embodiments, the presently disclosed subject matter provides a kit for screening at least one candidate agent that is capable of inhibiting growth or survival of bacteria from a selected genus, the kit comprising: (a) a population of isolated bacteria comprising bacteria from the selected genus or a culture thereof; (b) a staining mixture comprising: (i) a first agent which emits fluorescence of a first color that is indicative of live bacterial cells, and (ii) a second agent which emits fluorescence of a second color that contrasts from the first color and is indicative of dead bacterial cells, wherein when the staining mixture is incubated with the bacteria population or culture thereof a calculated ratio of the intensity of emitted fluorescence of the first color to the intensity of emitted fluorescence the second color is indicative of the percentage of live bacteria in the population or culture thereof; and (c) instructions for using the bacteria in (a) and the staining mixture in (b) to screen for at least one candidate agent that is capable of inhibiting growth or survival of bacteria from the selected genus.

In some embodiments, the kit further comprises at least one test agent to screen for its ability to inhibit the growth or survival of bacteria from a selected genus. In other embodiments, the kit further comprises instructions for contacting the population of bacteria or population thereof with at least one test agent. In still other embodiments, the kit further comprises instructions for incubating the staining mixture with the population of bacteria or culture thereof. In further embodiments, the kit further comprises instructions for assessing the viability of the bacteria in the population or culture thereof. In still further embodiments, the kit further comprises instructions for calculating a ratio of the intensity of emitted fluorescence of the first color to the intensity of emitted fluorescence the second color.

In some embodiments, the calculated intensity ratio in is indicative of the percentage of live bacteria in the population or culture thereof after a period of exposure to at least one test agent. In particular embodiments, the bacteria are Borrelia burgdorferi. In other embodiments, the bacteria are selected from the group consisting of Staphylococcus aureus, Escherichia coli, Klebsiella pneumoniae, Acinetobacter baumanii, and Mycobacterium tuberculosis.

In still other embodiments, the culture comprises a stationary phase culture comprising non-replicating persister cells. In further embodiments, the stationary phase culture comprises at least one morphological form selected from the group consisting of round bodies, planktonic, biofilm and combinations thereof.

In some embodiments, the first agent emits green fluorescence and the second agent emits red or orange fluorescence. In other embodiments, the first agent is SYBR Green I and the second agent is propidium iodide. In still other embodiments, the kit further comprises instructions for using SYBR Green I in the screen at a concentration of about 10× and using propidium iodide in the screen at a concentration of about 2 mM.

In some embodiments, the presently disclosed subject matter provides a kit for assessing the viability and sensitivity of cultures of bacteria from a selected genus for at least one agent (e.g., current Lyme disease antibiotics or any new agents) that is capable of inhibiting growth or survival of bacteria from the selected genus, the kit comprising cultures of bacteria from the selected genus, reagents for performing a SYBR Green I/Propidium iodide assay, and optionally at least one test agent.

### III. Methods for Inhibiting the Growth and/or Survival of Bacteria from the Borrelia Genus and Other Bacteria

The presently disclosed subject matter provides methods for killing, inhibiting, and/or preventing the growth of bacterial cells. In some embodiments, the presently disclosed subject matter provides a method for inhibiting the growth and/or survival of bacteria from a selected genus, the method comprising contacting bacteria from the selected genus with an effective amount of: (a) at least one compound selected from the group consisting of daptomycin, artemisinin, ciprofloxacin, sulfacetamide, sulfamethoxypyridazine, nifuroxime, fosfomycin, chlortetracycline, sulfathiazole, clofazimine, cefmenoxime, cefoperazone, carbomycin, cefotiam, cefepime, amodiaquin, fosfomycin and streptomycin; (b) at least one compound selected from the group consisting of daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; (c) a combination of at least two compounds comprising: (i) a first compound selected from the group consisting of daptomycin, cefoperazone, miconazole and sulfamethoxypyridazine; and (ii) a second compound other than the first compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; or (d) a combination of at least three compounds comprising: (i) doxycycline as a first compound; (ii) a second compound selected from the group consisting of daptomycin or cefoperazone; and (iii) a third compound other than the second compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone. In other embodiments, the compounds in (c) (ii) and (d) (iii) can be any currently used first-line treatment for Lyme disease. In still other embodiments, the presently disclosed method comprises administering any combination of the compounds in (a), (b), (c), and (d), e.g., at least two compounds comprising a first compound selected from (a), (b), (c) and (d), and a second compound other than the first selected from (a), (b), (c) and (d), at least three compounds comprising a first compound selected from (a), (b), (c) and (d), a second compound other than the first compound selected from (a), (b), (c) and (d), and a third compound other than the first or second compound selected from (a), (b), (c) and (d), etc.

In some embodiments, at least one compound in (b) is selected from the group consisting of daunomycin 3-oxime, dimethyldaunomycin, daunorubicin, 9,10-anthracenedione, 1-hydroxy-4-[[2-[(2-hydroxyethyl)amino]ethyl]amino]-, anthracene-9,10-dione, 1,5-bis[3-[[(2-hydroxyethyl)amino]propyl]amino]-9,10-dihydro-, and nogalamycin. In other embodiments, the combination of at least two compounds in (c) is daptomycin and doxycycline. In still other embodiments, the combination of at least two compounds in (c) is daptomycin and cefoperazone.

In some embodiments, the combination of at least three compounds in (d) is daptomycin, doxycycline, and cefoperazone. In still further embodiments, the combination of at least three compounds in (d) is daptomycin, doxycycline, and sulfamethoxypyridazine. In other embodiments, the combination of at least three compounds in (d) is daptomycin, doxycycline, and clofazamine. In still other embodiments, the combination of at least three compounds in (d) is daptomycin, doxycycline, and carbencillin. In further embodiments, the combination of at least three compounds in (d) is cefoperazone, doxycycline, and clofazamine. In still further embodiments, the combination of at least three compounds in (d) is cefoperazone, doxycycline, and sulfamethoxypyridazine. In other embodiments, the combination of at least three compounds in (d) is cefoperazone, doxycycline, and miconazole.

In some embodiments, the bacteria are Borrelia burgdorferi. In other embodiments, the bacteria comprise replicating forms of Borrelia burgdorferi, non-replicating persister forms of Borrelia burgdorferi, and combinations of replicating forms of Borrelia burgdorferi, non-replicating persister forms of Borrelia burgdorferi. In still other embodiments, the bacteria comprise a morphological form of Borrelia burgdorferi selected from the group consisting of round bodies, planktonic, and biofilm. In other embodiments, the bacteria are selected from the group consisting of Staphylococcus aureus, Escherichia coli, Klebsiella pneumoniae, Acinetobacter baumanii, and Mycobacterium tuberculosis. In further embodiments, the contacting occurs in vitro or in vivo.

The term “contacting” as used herein refers to any action that results in at least one compound of the presently disclosed subject matter physically contacting at least one bacterial cell or the environment in which at least one bacterial cell resides (e.g., a culture medium).

### IV. Methods for Treating Lyme Disease

In some embodiments, the presently disclosed subject matter provides methods for treating Lyme disease, for example in a subject that has post-treatment Lyme disease syndrome (PTLDS) and/or antibiotic refractory Lyme arthritis. It has been found that an effective amount of particular antibiotics in combination with an effective amount of at least one other particular antibiotic is able to kill non-replicating persister cells. In other embodiments, the method inhibits a bacterial infection in a subject, such as a Borrelia burgdorferi infection.

Accordingly, in some embodiments, the presently disclosed subject matter provides a method for treating Lyme disease in a subject in need thereof, the method comprising administering to a subject an effective amount of: (a) at least one compound selected from the group consisting of daptomycin, artemisinin, ciprofloxacin, sulfacetamide, sulfamethoxypyridazine, nifuroxime, fosfomycin, chlortetracycline, sulfathiazole, clofazimine, cefmenoxime, cefoperazone, carbomycin, cefotiam, cefepime, amodiaquin, fosfomycin and streptomycin; (b) at least one compound selected from the group consisting of daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; (c) a combination of at least two compounds comprising: (i) a first compound selected from the group consisting of daptomycin, cefoperazone, miconazole and sulfamethoxypyridazine; and (ii) a second compound other than the first compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; or (d) a combination of at least three compounds comprising: (i) doxycycline as a first compound; (ii) a second compound selected from the group consisting of daptomycin or cefoperazone; and (iii) a third compound other than the second compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone. In other embodiments, the compounds in (c) (ii) and (d) (iii) can be any currently used first-line treatment for Lyme disease. In still other embodiments, the presently disclosed method comprises administering any combination of the compounds in (a), (b), (c), and (d).

In some embodiments, at least one compound in (b) is selected from the group consisting of daunomycin 3-oxime, dimethyldaunomycin, daunorubicin, 9,10-anthracenedione, 1-hydroxy-4-[[2-[(2-hydroxyethyl)amino]ethyl]amino]-, anthracene-9,10-dione, 1,5-bis[3-[[(2-hydroxyethyl)amino]propyl]amino]-9,10-dihydro-, and nogalamycin. In other embodiments, the combination of at least two compounds in (c) is daptomycin and doxycycline. In still other embodiments, the combination of at least two compounds in (c) is daptomycin and cefoperazone.

In some embodiments, the combination of at least three compounds in (d) is daptomycin, doxycycline, and cefoperazone. In other embodiments, the combination of at least three compounds in (d) is daptomycin, doxycycline, and sulfamethoxypyridazine. In still other embodiments, the combination of at least three compounds in (d) is daptomycin, doxycycline, and clofazamine. In further embodiments, the combination of at least three compounds in (d) is daptomycin, doxycycline, and carbencillin. In still further embodiments, the combination of at least three compounds in (d) is cefoperazone, doxycycline, and clofazamine. In some embodiments, the combination of at least three compounds in (d) is cefoperazone, doxycycline, and sulfamethoxypyridazine. In other embodiments, the combination of at least three compounds in (d) is cefoperazone, doxycycline, and miconazole.

In some embodiments, the bacteria are Borrelia burgdorferi. In other embodiments, the bacteria comprise replicating forms of Borrelia burgdorferi, non-replicating persister forms of Borrelia burgdorferi, and combinations of replicating forms of Borrelia burgdorferi, non-replicating persister forms of Borrelia burgdorferi. In still other embodiments, the bacteria comprise a morphological form of Borrelia burgdorferi selected from the group consisting of round bodies, planktonic, and biofilm. In further embodiments, the subject has, or is suspected of having, post-treatment Lyme disease syndrome (PTLDS) and/or antibiotic refractory Lyme arthritis.

In some embodiments, the presently disclosed subject matter provides a method for treating Lyme disease in a subject in need thereof, the method comprising: (a) administering to the subject an effective amount of a combination of at least two agents comprising: (i) at least one agent that inhibits growth and/or survival of replicating forms of bacteria from the Borrelia genus; and (ii) at least one agent that inhibits growth and/or survival of non-replicating persister forms of bacteria from the Borrelia genus. In other embodiments, the method further comprises one or more steps selected from the group consisting of: (b) obtaining from the subject a biological sample comprising one or more morphological forms of bacteria from the Borrelia genus; (c) isolating at least one of the morphological forms of the bacteria; (d) culturing the isolated bacteria; and (e) assessing the susceptibility of the cultured bacteria to the at least one agent that inhibits the growth and/or survival of replicating forms of bacteria, the at least one agent that inhibits the growth and/or survival of non-replicating persister forms of bacteria, or both.

In some embodiments, at least one agent that inhibits growth and/or survival of replicating forms of bacteria is selected from the group consisting of a beta-lactam, an antibiotic that damages DNA, and an energy inhibitor. In other embodiments, at least one agent that inhibits the growth and/or survival of replicating forms of bacteria is incubated in vitro with a culture comprising non-replicating persister forms of bacteria from the Borrelia genus, at least one agent that inhibits growth and/or survival of replicating forms of bacteria inhibits the growth and/or survival of less than 25 percent of the population of non-replicating persister bacteria in the culture. In still other embodiments, at least one agent that inhibits the growth and/or survival of replicating forms of bacteria is selected from the group consisting of doxycycline, cefoperazone, carbenicillin, clofazimine, and combinations thereof. In further embodiments, at least one agent that inhibits the growth and/or survival of non-replicating persister forms of bacteria is an anthraquinone-containing compound. In still further embodiments, at least one agent that inhibits the growth and/or survival of non-replicating persister forms of bacteria is incubated in vitro with a culture comprising non-replicating persister forms of bacteria from the Borrelia genus, the at least one agent that inhibits growth and/or survival of the non-replicating persister forms of bacteria inhibits the growth and/or survival of greater than about 50 percent of the population of non-replicating persister forms of bacteria in the culture. In some embodiments, at least one agent inhibits the growth and/or survival of greater than about 75 percent of the population of non-replicating persister forms of bacteria in the culture. In some embodiments, at least one agent inhibits the growth and/or survival better than the current antibiotics used for Lyme disease, such as doxycycline, amoxicillin, cefuroxime or ceftriaxone, metronidazole, tinidazole, and combinations thereof. In other embodiments, at least one agent that inhibits the growth and/or survival better than the current antibiotics used for Lyme disease can be determined using the presently disclosed methods.

In some embodiments, at least one agent that inhibits the growth and/or survival of non-replicating persister forms of bacteria is selected from the group consisting of daptomycin, artemisinin, ciprofloxacin, sulfacetamide, sulfamethoxypyridazine, nifuroxime, fosfomycin, chlortetracycline, sulfathiazole, clofazimine, cefmenoxime, cefoperazone, carbomycin, cefotiam, cefepime, amodiaquin, fosfomycin and streptomycin; daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone.

In some embodiments, the bacteria are Borrelia burgdorferi. In other embodiments, the bacteria comprise replicating forms of Borrelia burgdorferi, non-replicating persister forms of Borrelia burgdorferi, and combinations of replicating forms of Borrelia burgdorferi, non-replicating persister forms of Borrelia burgdorferi. In still other embodiments, the bacteria comprise a morphological form of Borrelia burgdorferi selected from the group consisting of round bodies, planktonic, and biofilm.

As used herein, the term “antibiotic” refers to a compound that has the ability to kill or inhibit the growth of bacteria, particularly bacteria selected from a genus including, but not limited to, Borrelia, Staphylococcus, Escherichia, Klebsiella, Acinetobacter, and Mycobacterium. More generally, an antimicrobial is an agent that kills microorganisms or inhibits their growth. Antimicrobials medicines can be grouped according to the microorganisms they act primarily against. For example, antibiotics are used against bacteria. As used herein, the term ‘beta-lactam” or “beta-lactam antibiotic” refers to an antibiotic with a beta-lactam ring as part of its core structure, such as penicillin and penicillin derivatives (penams), cephalosporins (cephems), monobactams, and carbapenems. Many of these antibiotics work by inhibiting bacterial cell wall biosynthesis.

In some embodiments, the subject has, or is suspected of having, post-treatment Lyme disease syndrome (PTLDS) and/or antibiotic refractory Lyme arthritis. The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

In some embodiments, the term “effective amount” refers to the amount of antibiotic or compound required to inhibit or kill a bacterial cell. In other embodiments, the term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy. In particular embodiments, the disease, disorder, or condition is Lyme disease.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. The terms “treat,” “treating,” “treatment,” and the like, as used herein can refer to curative therapy, prophylactic therapy, and preventative therapy. The treatment, administration, or therapy can be consecutive or intermittent. Consecutive treatment, administration, or therapy refers to treatment on at least a daily basis without interruption in treatment by one or more days. Intermittent treatment or administration, or treatment or administration in an intermittent fashion, refers to treatment that is not consecutive, but rather cyclic in nature. Treatment according to the presently disclosed methods can result in complete relief or cure from a disease, disorder, or condition, or partial amelioration of one or more symptoms of the disease, disease, or condition, and can be temporary or permanent. The term “treatment” also is intended to encompass prophylaxis, therapy and cure.

The term “combination” is used in its broadest sense and means that a subject is administered at least two agents, for example an antibiotic, and one or more antibacterial agents. More particularly, the term “in combination” refers to the concomitant administration of two (or more) active agents for the treatment of a, e.g., single and multiple disease states with heterogeneous bacterial populations consisting of growing and non-growing or any in between bacterial cells. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one, but not the other). The single dosage form may include additional active agents for the treatment of the disease state.

Further, the compounds described herein can be administered alone or in combination with adjuvants that enhance stability of the compounds, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the compounds can be varied so long as the beneficial effects of the combination of these agents are achieved. Accordingly, the phrase “in combination with” refers to the administration of a compound, and at least one additional therapeutic agent, such as an antibiotic or other compound, either simultaneously, sequentially, or a combination thereof. Therefore, a subject administered a combination of a compound and at least one additional therapeutic agent, can receive the compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### V. GENERAL DEFINITIONS

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Methods for Identifying Novel Anti-Persister Activity for Borrelia burgdorferi

### Materials and Methods

Bacterial Strain, Media and Culture:

Borrelia burgdorferi strain B31 was obtained from the American Type Tissue Collection. Borrelia burgdorferi was cultured in BSK-H medium (HiMedia Laboratories Pvt. Ltd.), with 6% rabbit serum (Sigma-Aldrich, St. Louis, Mo.). All culture media were filter-sterilized by 0.2-μm filter. Cultures were incubated in sterile 50-mL closed conical tubes (BD Biosciences, San Diego, Calif.) at 33° C. without antibiotics. After 7 days, 100-μL stationary-phase B. burgdorferi cultures (1×10⁶ cells) were transferred to 96-well tissue culture microplates for drug screening.

Microscopy Techniques:

Specimens were examined on a Nikon Eclipse E800 microscope equipped with differential interference contrast (DIC) and epi-fluorescent illumination, and recorded with a Spot slider color camera. Cell proliferation assays were performed by directly counting using a bacterial counting chamber (Hausser Scientific Partnership, Horsham, Pa.) and DIC microscopy. To assay the viability of B. burgdorferi, the SYBR Green I/PI assay or LIVE/DEAD BacLight bacterial viability assay was performed. The ratio of live (green) and dead (red) B. burgdorferi was calculated by counting these cells using a bacterial counting chamber and epi-fluorescent microscopy.

Antibiotics and FDA-Approved Drug Library:

Doxycycline, amoxicillin, metronidazole, clofazimine, ketoconazole, miconazole, aspirin, polymyxin B (PMB), and sulfamethoxazole (SMX) (all purchased from Sigma-Aldrich) were dissolved in appropriate solvents (Clinical and Laboratory Standards Institute, 2007) to form stock solutions. All the antibiotic stocks were filter-sterilized by a 0.2-μm filter. The stocks then were pre-diluted into 500-μM pre-diluted stocks and stored at −20° C.

Each drug in the Johns Hopkins Clinical Compound Library (JHCCL) (Chong et al., 2006) was made into 10-mM stock solutions with DMSO. The stock solutions were arrayed in a total of 24 96-well plates, leaving the first and the last columns in each plate for controls. Each solution in these master plates was diluted with PBS to make 500-μM pre-diluted plates. The first and the last columns in each pre-diluted plate were included as blank controls, doxycycline control, and amoxicillin control. The pre-diluted drug plates were sealed and stored at −20° C.

Antibiotic Susceptibility Test:

To qualitatively determine the effect of the antibiotics, 10 μL of each compound from the pre-diluted plate or pre-diluted stock was added to the B. burgdorferi culture in the screening plate. The final volume per well was adjusted to 100 μL. The plates were sealed and placed in a 33° C. incubator for 7 days.

For assaying the live and dead cells in the screening plates, the SYBR Green I/PI assay was used as described in a previous study (Feng, Wang, Shi, et al., 2014). SYBR Green I (10,000×stock, Invitrogen, Carlsbad, Calif.) (10 μL) was mixed with 30 μL propidium iodide (20 mM, Sigma-Aldrich) into 1.0 mL of sterilized dH₂O and mixed thoroughly. Staining mixture (10 μL) was added to each well and mixed thoroughly. The plates at room temperature were incubated in the dark for 15 minutes. With the excitation wavelength at 485 nm, the fluorescence intensities at 535 nm (green emission) and 635 nm (red emission) were measured for each well of the screening plate using a HTS 7000 plus Bio Assay Reader (PerkinElmer Inc., Waltham, Mass.). Meanwhile the B. burgdorferi suspensions (live and 70% isopropyl alcohol-killed) were mixed with five different proportions of live:dead cells (0:10, 2:8, 5:5, 8:2, 10:0) the mixture was added in wells of a 96-well plate. SYBR Green I/PI reagent was then added to each of the five samples and the green/red fluorescence ratios for each proportion of live/dead B. burgdorferi were measured using a HTS 7000 plus Bio Assay Reader as described above. The regression equation and regression curve of the relationship between the percentage of live bacteria and green/red fluorescence ratios were obtained with least-square fitting analysis. The regression equation was used to calculate the percentage of live cells in each well of the screening plate. Some effective candidates were further confirmed by epi-fluorescence microscope counting.

MIC Determination:

The standard microdilution method was used to determine the antibiotic minimum inhibitory concentration (MIC) that would inhibit visible growth of B. burgdorferi after a 72-hour incubation period (Sapi et al., 2011; Dever et al., 1992; Boerner et al., 1995). B. burgdorferi cells (1×10⁵) were inoculated into each well of a 96-well microplate containing 90 μL fresh BSK-H medium per well. Each diluted antibiotic (10 μL) was added to the culture. All experiments were run in triplicate. The 96-well plate was sealed and placed in the incubator at 33° C. for 5 days. Cell proliferation was assessed using a SYBR Green I/PI assay and a bacterial counting chamber after the incubation.

Establishment of a Stationary Phase Model for Drug Screens:

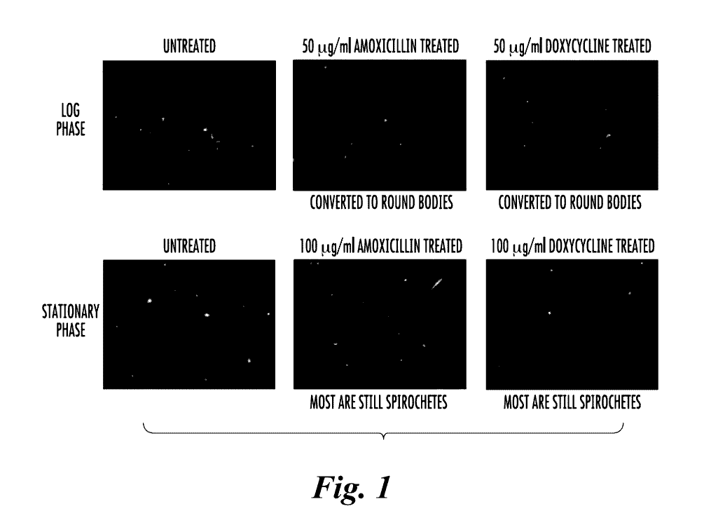

Representative morphological changes of Borrelia cells following treatment with some currently used antibiotics is shown in FIG. 1. During the log phase, the Borrelia cells treated with amoxicillin and doxycycline adopt cystic or round body forms. During the stationary phase, the Borrelia cells treated with the same antibiotic adopt a spirochete form. These morphological variants of B. burgdorferi have different antibiotic susceptibilities.

### Results

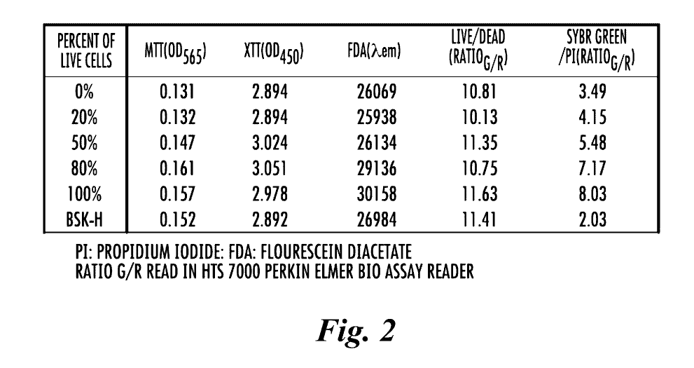

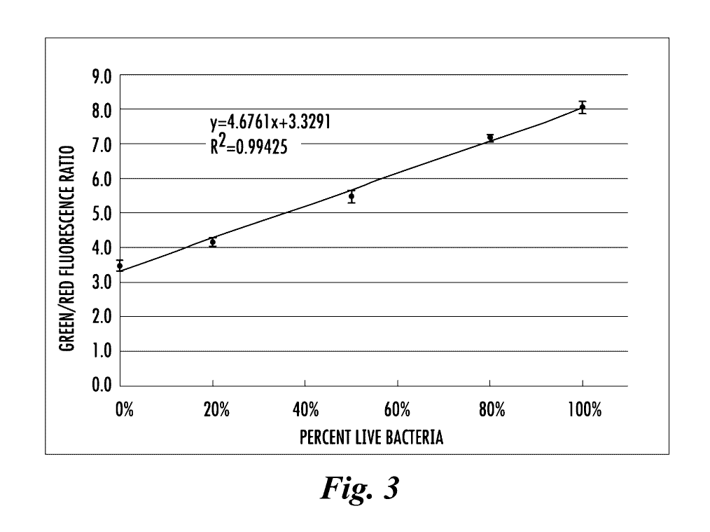

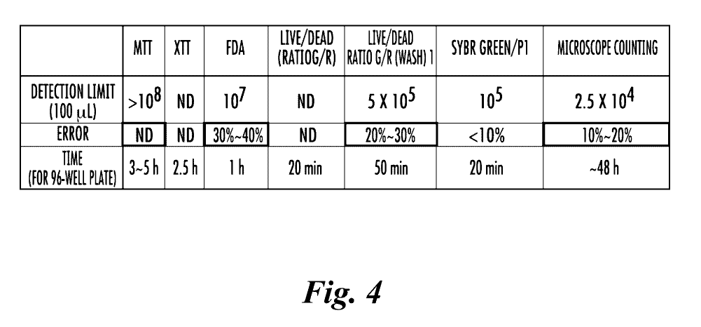

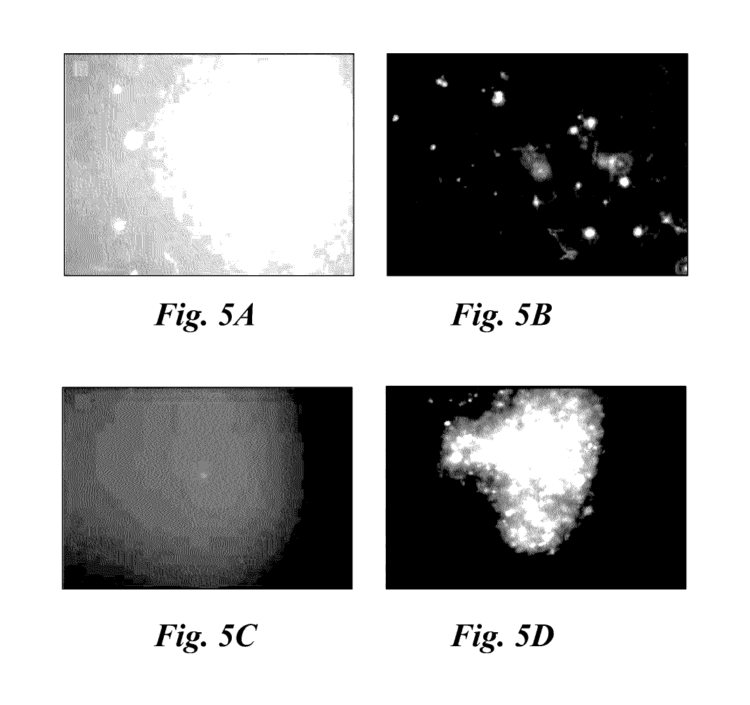

A comparison of methods for assaying B. burgdorferi viability in a 96-well plate showed that the SYBR Green/propidium iodide (PI) assay using the green fluorescence to red fluorescence ratio resulted in a consistent correlation between the percent of live cells and the ratio (FIG. 2). When plotted, this was found to be a linear ratio (FIG. 3).

FIG. 4 shows the B. burgdorferi B31 strain with commonly used viability assays MTT, XTT, fluoroscein diacetate assay (FDA), the commercially available LIVE/DEAD BacLight assay, and the presently disclosed SYBR Green I/PI assay. The SYBR Green I/PI assay had a less than 10% error and could be completed in approximately 20 minutes.

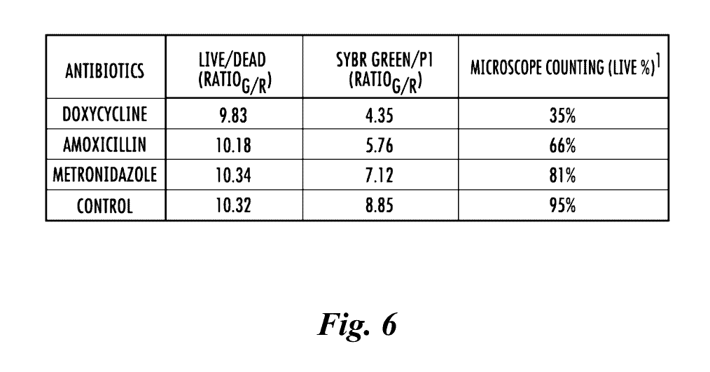

FIG. 5A, FIG. 5B, FIG. 5C, and FIG. 5D show the B. burgdorferi B31 strain observed with: (FIG. 5A) the fluorescent microscopy LIVE/DEAD BacLight stain; (FIG. 5B) SYBR Green/PI stain; (FIG. 5C) FDA stain; and (FIG. 5D) the B. burgdorferi biofilm stained by SYBR Green/PI. The SYBR Green/PI assay showed correlation with direct microscope counting with antibiotic exposure (FIG. 6).

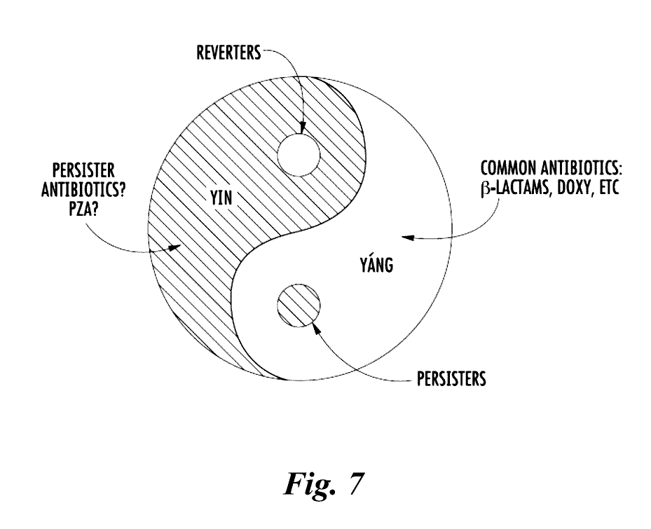

FIG. 7 shows a representative drawing of the Yin-Yang model of bacterial persisters and latent infections (Zhang, 2014). The Yin-Yang model depicts a dynamic and complex bacterial population consisting of growing (Yang, in red) and non-growing populations (Yin, in black) which are in varying metabolic states in continuum and can interconvert. In the growing population (Yang), there is a small population of non-growing or slowly growing persisters (Yin), which in turn contain a small number of growing bacteria. The persister population is again heterogeneous and composed of various sub-populations in continuum and includes a varying hierarchy of persisters. In the case of TB, isoniazid (INH) kills growing bacteria (Yang), rifampin (RIF) kills some growing bacteria and slowly growing persisters, whereas pyrazinamide (PZA) kills only persisters. Persisters not killed by antibiotics could revert to replicating forms (reverters) and cause relapse. The current Lyme disease antibiotics doxycycline and amoxicillin or ceftriaxone only kill the growing Borrelia burgdorferi bacteria (Yang) and have little or no activity for dormant non-growing Borrelia burgdorferi persisters (Yin). The Yin-Yang model proposes targeting both replicating and non-replicating cells for better treatment of both persistent bacterial infections, including Lyme disease. For example, newly identified persister active drugs or antibiotics, such as daptomycin, clofazimine, cefoperazone, carbomycin, sulfa drugs, and/or quinolones, can be used in combination with currently used Lyme disease antibiotics, such as doxycycline, amoxicillin and/or ceftriaxone, which are active against the growing Borrelia burgdorferi bacteria (Yang) for more effective treatment of all forms of Lyme disease, especially the chronic and persistent forms of the disease.

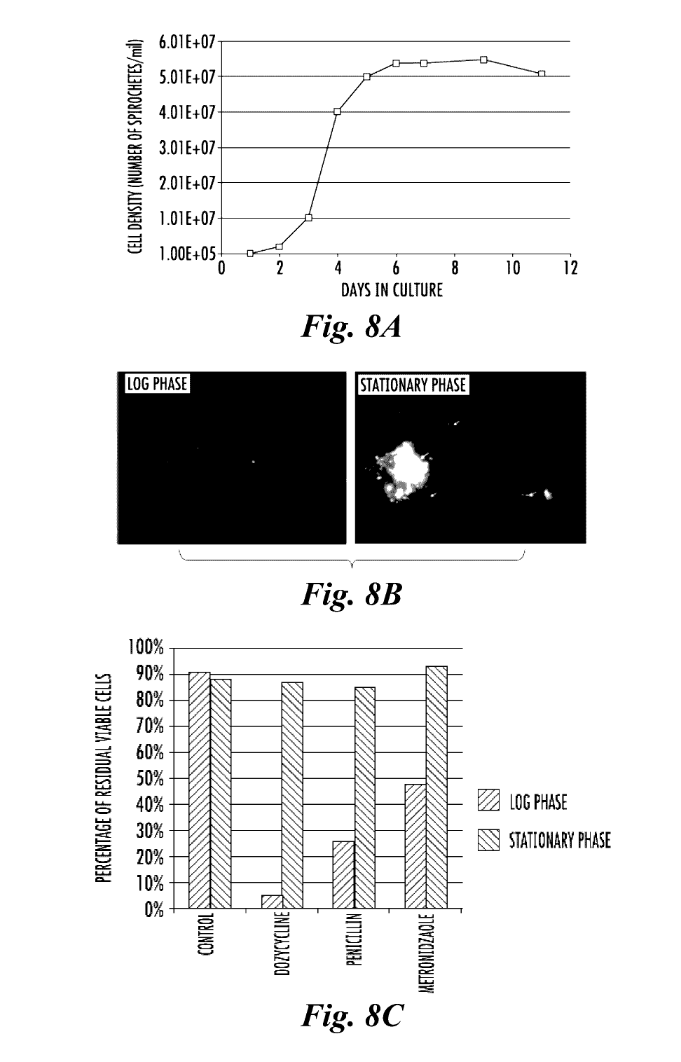

For FIG. 8, B. burgdorferi culture was grown in BSK-H medium for 7 days, and cell number was determined by microscope counting at different time points. The B. burgdorferi growth reached peaks (5×10⁷ spirochetes/mL) after 5-6 days. The microscope counts of cell density remained relatively constant until 11 days of incubation (FIG. 8A). Studies showed that persistent B. burgdorferi might be in different morphological forms, namely round bodies (cysts) and biofilm. These multiple morphological forms of B. burgdorferi might have different antibiotic susceptibilities (Brorson et al., 2009; Sapi et al., 2011). By microscope examination, the ratio of round bodies and biofilm-like colonies significantly increased in the stationary phase B. burgdorferi culture (FIG. 8B). These stationary phase cultures may represent a mixed population of different morphological forms. The B. burgdorferi stationary phase culture of 7 days was chosen as a persistence model to screen for drugs.

Evaluation of In Vitro Antibiotic Susceptibility of Stationary Phase B. burgdorferi:

Previous studies and clinical experiences demonstrated that doxycycline and amoxicillin exhibit bactericidal activity againstB. burgdorferi (Hunfeld and Brade, 2006). The conventional antibiotics used for Lyme disease, such as doxycycline and penicillin, do not kill the cystic form of B. burgdorferi, yet some studies showed that metronidazole could kill the cystic form of B. burgdorferi (Brorson and Brorson, 1999). The presently disclosed subject matter discloses the effect of these frontline drugs (doxycycline, amoxillin and metronidazole) on log phase and stationary phase B. burgdorferi and evaluation and susceptibility by the SYBR Green I/PI assay. Treatment with clinical commonly used antibiotics showed these frontline drugs were effective against log phase B. burgdorferi, but had little effect against stationary phase B. burgdorferi (FIG. 8C). The result of microscope counting correlated with the result of the SYBR Green I/PI assay.

Screening FDA-Approved Drug Library for Effective Drugs Against Dormant B. burgdorferi:

For screening the effect of antibiotics on persister B. burgdorferi, stationary phase B. burgdorferi were used as a persistence model to screen an FDA-approved drugs library. Meanwhile, doxycycline and amoxicillin were added to each test plate as control drugs. Several measurements for the activity of control drugs revealed that the relative error of using the SYBR Green I/PI assay was less than 15%. Of the 1,514 drugs tested, dozens of antibiotics were considered to have a higher activity than the clinical commonly used antibiotics against the B. burgdorferi persisters (Table 1). Epi-fluorescence microscope counting further validated some effective candidates measured by SYBR Green I/PI, and the agreement of them was good with the largest difference less than 20%.

TABLE 1
Activity of top 27 active hits that had good activity (better than current
clinical drugs) against stationary phase B. burgdorferi persisters ^{a}

| | Residual | Residual | Ratio of Green/Red fluoresce | | | | |
|---|---|---|---|---|---|---|---|
| | viable | viable | Primary | | | | |
| Drugs (50 μM) | cells^{b} | cells^{c} | screening | Rescreening | Rescreening | p-value^{d} | p-value^{e} |
| Control | 93% | 94% | 8.67 | 8.38 | 8.59 | — | — |
| Amoxicillin | 76% | 76% | 7.98 | 7.86 | 7.82 | 1.000000 | 0.2336 |
| Doxycycline | 75% | 67% | 7.62 | 7.35 | 7.58 | 0.233596 | 1.0000 |
| Penicillin G | 75% | 68% | 7.41 | 7.68 | 7.92 | 0.699416 | 0.3987 |
| Tetracycline | 54% | 50% | 7.59 | 6.14 | 7.18 | 0.102366 | 0.1712 |
| Ceftriaxone | 50% | 44% | 6.74 | 6.89 | 6.78 | 0.000182 | 0.0029 |
| Cefuroxime | 49% | 43% | 6.59 | 6.84 | 6.67 | 0.000317 | 0.0029 |
| Clarithromycin | 70% | 65% | 7.70 | 7.36 | 7.59 | 0.038775 | 0.3422 |
| Azithromycin | 77% | 80% | 8.33 | 8.10 | 7.92 | 0.071492 | 0.0703 |
| Daptomycin | 35% | 28% | 6.10 | 6.20 | 6.09 | 0.000008 | 0.0002 |
| Clofazimine | 45% | 32% | 6.56 | 6.23 | 6.02 | 0.000599 | 0.0017 |
| Cefoperazone | 37% | 34% | 6.54 | 6.32 | 6.23 | 0.000126 | 0.0008 |
| Carbomycin | 41% | 37% | 6.37 | 6.81 | 6.32 | 0.001045 | 0.0033 |
| Vancomycin | 48% | 38% | 6.65 | 6.58 | 6.37 | 0.000152 | 0.0011 |
| Cephalothin | 49% | 40% | 6.74 | 6.49 | 6.55 | 0.000133 | 0.0012 |
| Cefotiam | 42% | 43% | 6.41 | 7.55 | 6.21 | 0.000503 | 0.0840 |
| Control | 93% | 94% | 8.67 | 8.38 | 8.59 | — | — |
| Cefmetazole | — | 43% | 6.80 | 7.38 | 6.00 | 0.045064 | 0.0767 |
| Cefepime | — | 44% | 6.67 | 7.16 | 6.45 | 0.006368 | 0.0162 |
| Amodiaquin | — | 45% | 6.79 | 6.44 | 6.85 | 0.000946 | 0.0040 |
| Streptomycin | — | 45% | 6.72 | 6.93 | 6.76 | 0.000175 | 0.0022 |
| Ticarcillin | — | 46% | 6.82 | 6.72 | 6.93 | 0.000163 | 0.0023 |
| Cefonicid | — | 46% | 6.86 | 7.54 | 6.07 | 0.067661 | 0.1130 |
| Piperacillin - | 47% | 47% | 7.18 | 6.47 | 6.98 | 0.009594 | 0.0253 |
| tazobactam | | | | | | | |
| Cefdinir | — | 48% | 6.88 | 7.51 | 6.29 | 0.049107 | 0.0911 |
| Ceforanide | — | 48% | 6.89 | 7.49 | 6.33 | 0.043847 | 0.0839 |
| Cefmenoxime | — | 48% | 6.82 | 7.59 | 6.32 | 0.058674 | 0.1062 |
| Bismuth | — | 48% | 6.94 | 6.82 | 6.92 | 0.000082 | 0.0024 |
| Ceftizoxime | — | 49% | 6.94 | 6.83 | 7.03 | 0.000223 | 0.0036 |
| Ceftibuten | 51% | 49% | 6.81 | 6.78 | 7.27 | 0.004888 | 0.0177 |
| Amphotericin B | — | 50% | 7.14 | 6.88 | 6.87 | 0.000783 | 0.0065 |
| Cefamandole | — | 50% | 6.71 | 7.73 | 6.52 | 0.076304 | 0.1378 |
| Quinine | — | 50% | 7.00 | 6.85 | 6.88 | 0.000124 | 0.0028 |
| hydrobromide | | | | | | | |
| Cyclacillin | 51% | 53% | 6.81 | 6.88 | 7.64 | 0.045210 | 0.1052 |
| Collistin | 50% | 54% | 7.15 | 7.26 | 7.23 | 0.000319 | 0.0135 |
| Sulfameter | | 54% | 7.13 | 7.46 | 6.98 | 0.009635 | 0.0451 |
| Tigecycline | 58% | 51% | 6.98 | 7.06 | 6.96 | 0.001557 | 0.0138 |

^{a} Stationary phase B. burgdorferi (7-day old) cells were treated with drugs for 7 days.
^{b}Residual viable B. burgdorferi was assayed by epifluorescence microscope counting.
^{c}Residual viable B. burgdorferi was calculated according to the regression equation and ratio of Green/Red fluorescence obtained by SYBR Green I/PI assay.
^{d}the p-value of standard T-test for treated group and amoxicillin treated samples.
^{e}the p-value of standard T-test for treated group and doxycycline treated samples.

Based on primary screening, some active candidates were selected (with residual viable cells less than 50%) for re-screening by SYBR Green I/PI assay and microscope counting. The re-screen confirmed the result of primary screening.

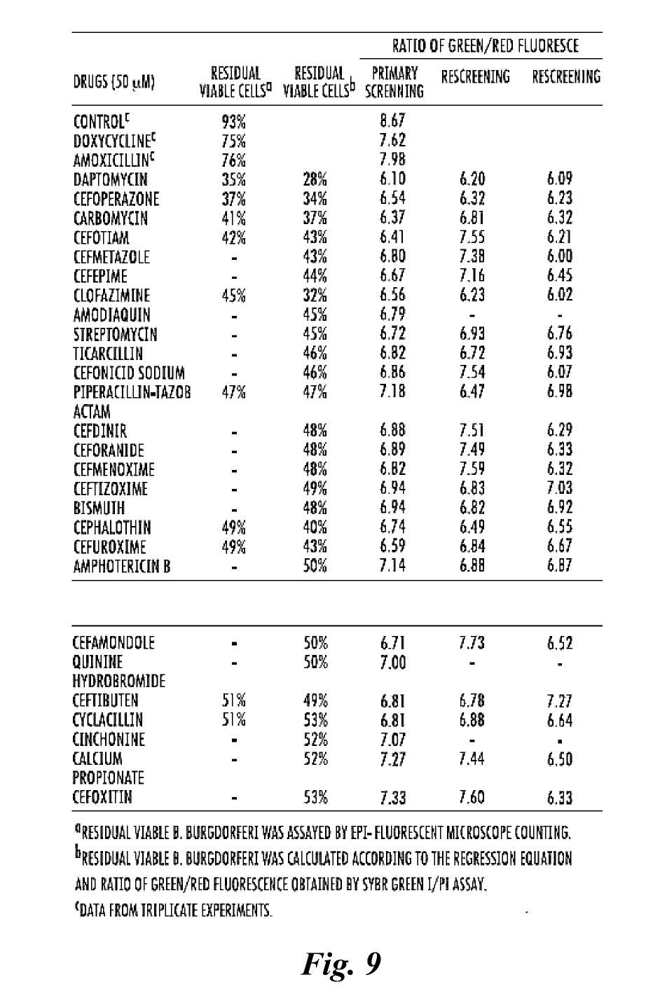

Several FDA-approved drugs were identified showing good bactericidal activity against stationary phase B. burgdorferi. Bactericidal activities of some drugs were significantly higher than that of frontline antibiotics doxycycline or amoxicillin (FIG. 9). For example, daptomycin, clofazimine, carbomycin, some cephalosporin antibiotics (such as cefoperazone, cefotiam and cefepime), streptomycin, and antimalarial antibiotic amodiaquim, showed relatively high bactericidal activities against stationary phase B. burgdorferi.

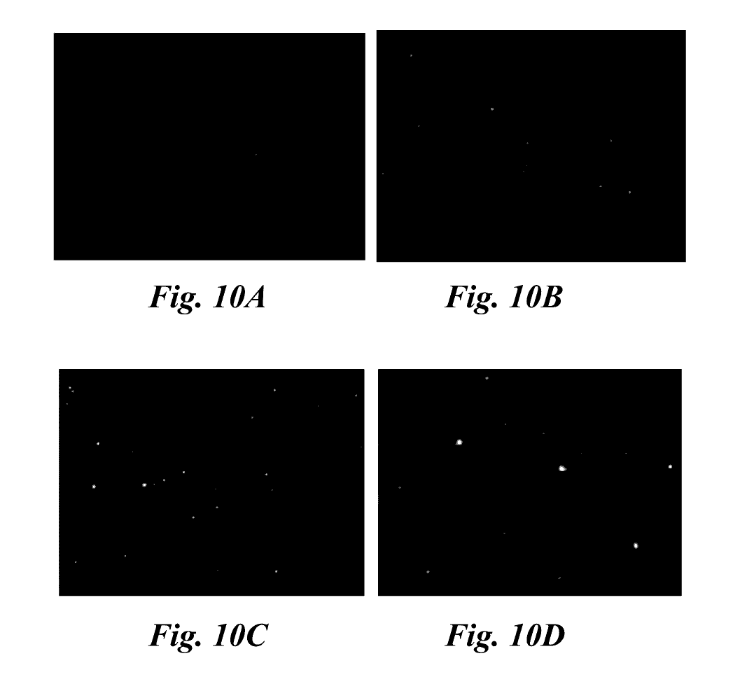

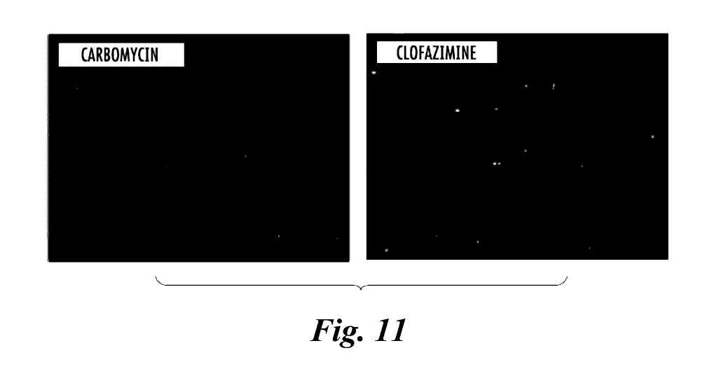

Representative images are shown of the stationary phase of B. burgdorferi strain B31 treated by daptomycin, cefoperazone, and tetracycline (FIG. 10A, FIG. 10B, FIG. 10C, and FIG. 10D), as well as carbomycin and clofazimine (FIG. 11). Some of these compounds may block protein synthesis. For example, macrolides have greater activity than penicillin or ceftriaxone for B. burgdorferi persisters. Carbomycin, a macrolide, showed higher activity against B. burgdorferi than other macrolides, such as erythromycin. Other compounds may damage DNA and/or energy, such as clofazimine. Compounds also might block DNA synthesis. Some sulfa drugs, such as sulfameter and sulfisoxazole, were effective against stationary phase B. burgdorferi, while sulfamethoxazole also exhibited a low MIC (≦0.2 g/mL).

Although most drugs did not affect the SYBR Green I/PI assay, some colored compounds caused interference to the SYBR Green I/PI assay. For example, pyrvinium pamoate and doxorubicin showed very high activity by the SYBR Green I/PI assay, but no bactericidal activity was observed by microscopic counting. It was found that these red compounds could make the background red and cause false positive results. Thus, validation by other methods, such as microscopic counting, to confirm the SYBR Green I/PI data is suggested.

MIC Test:

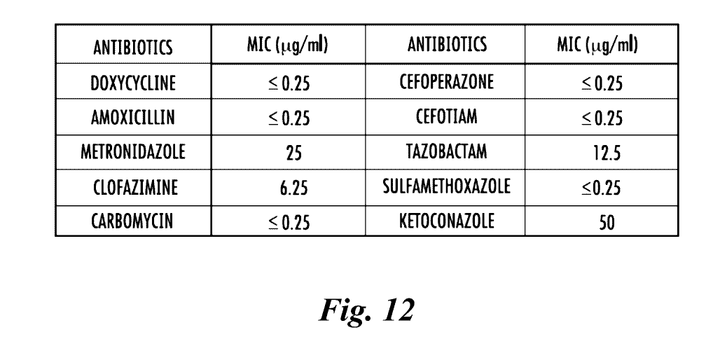

The MICs of some effective antibiotics against stationary phase B. burgdorferi were determined by the new SYBR Green I/PI assay and microscope counting. The results obtained from the two methods were the same. The MIC values (FIG. 12) of doxycycline, amoxicillin and metronidazole were in agreement with previous studies (Sapi et al., 2011; Hunfeld and Brade, 2006). Meanwhile it was found that log phase B. burgdorferi was very sensitive to carbomycin, cefoperazone, cefotiam and sulfamethoxazole (FIG. 12). On the other hand metronidazole, clofazimine, tazobactam, ketoconazole, miconazole were less potent against multiplying B. burgdorferi (FIG. 12).

The presently disclosed subject matter provides a rapid and convenient viability assay (e.g., SYBR Green I/PI) that is suitable for high-throughput screening for identifying new drugs and for rapidly evaluating antibiotic susceptibility of B. burgdorferi (Feng, Wang, Shi, et al., 2014). Using this rapid method, a FDA-approved compound library was screened for activity against non-replicating persisters of B. burgdorferi. A number of drug candidates were identified that have activity for Borrelia persisters.

Daptomycin is a lipopeptide antibiotic used in the treatment of infections caused by Gram-positive organisms. The presently disclosed data showed daptomycin had the highest activity against stationary phase B. burgdorferi persisters among all the active hits. Daptomycin could disrupt multiple aspects of bacterial cell membrane function. It inserts into the membrane, and creates pores that allow cells to leak ions, which causes rapid depolarization, resulting in a loss of membrane potential and bacterial cell death (Pogliano et al., 2012). The B. burgdorferi cells treated by daptomycin showed almost all red fluorescence as spirochetes (FIG. 10A) after staining. This result indicated the daptomycin could disrupt the cell membrane of B. burgdorferi, resulting in propidium iodide permeating into the cell. Microscope counting revealed few spheroplasts after daptomycin treatment; without wishing to be bound to any one particular theory, it is thought that the daptomycin could induce lysis of spheroplast cells.

Macrolides and ketolides were chosen as candidate antibiotics for clinical therapy of Lyme disease in previous studies (Hunfeld and Brade, 2006). Here, it has been found that carbomycin, a 16-membered macrolide, showed higher bactericidal activity against stationary phase B. burgdorferi than the classic macrolides, such as erythromycin and roxithromycin. The MIC data (FIG. 12) showed carbomycin also was effective against multiplying B. burgdorferi.

Treatment of B. burgdorferi with beta-lactams was commonly used therapy in a clinical setting (Hunfeld and Brade, 2006). Beta-lactams might induce round-body propagules of B. burgdorferi by disrupting the synthesis of the peptidoglycan layer of cell walls (Kersten et al., 1995). Microscopic examination also showed that round-body propagules (Brorsen et al., 2009) were the majority in the cefoperazone treated stationary phase B. burgdorferi (FIG. 10B). According to the MICs measured by previous studies, all beta-lactams showed good activity against multiplying B. burgdorferi. The presently disclosed subject matter shows, however, that a difference exists in the effects of beta-lactam antibiotics on stationary phase B. burgdorferi cells.

Cefoperazone, a third generation cephalosporin, appears to be the best beta-lactam antibiotic against stationary phase B. burgdorferi, followed by some second generation cephalosporins, such as cefotiam, cefmetazole and cefonicid. As in previous studies (Hunfeld and Brade, 2006), first generation cephalosporins showed very limited activity against stationary phase B. burgdorferi. It has been found that the activities of cephalosporins against stationary phase B. burgdorferi did not completely fit with the classic generation grouping of these antibiotics according to their spectrum of activity against Gram-negative and Gram-positive bacteria. This observation is probably related to the differences of B. burgdorferi from common gram-negative or gram-positive bacteria (Bergström and Zückert, 2010). Although beta-lactamase inhibitor tazobactam had a limited effect on multiplying B. burgdorferi, piperacillin-tazobactam was active against stationary phase B. burgdorferi. Genomic data showed B. burgdorferi possessed a gene coding putative beta-lactamase PhnP. Therefore, a beta-lactamase inhibitor might be helpful in reducing the resistance rates of B. burgdorferi to beta-lactams. In some embodiments, the optimized combination of beta-lactams and lactamase inhibitor has good activity against B. burgdorferi persistence.

Tetracycline antibiotics, especially doxycycline, are used in clinic settings as frontline drugs for Lyme disease. These antibiotics have lower MIC values (Hunfeld and Brade, 2006) and have good activity on multiplying B. burgdorferi. Interestingly, it was found that tetracycline had higher activity against stationary phase B. burgdorferi than doxycycline. It was observed that most cells were round-body propagules in the stationary phase B. burgdorferi treated by tetracycline (FIG. 10C). B. burgdorferi could form different morphological shapes in stationary phase or under adverse conditions, while tetracycline antibiotics might be ineffective on some morphological cells, such as round bodies (cysts) cells (Sapi et al., 2011).

In addition, some FDA-approved antibiotics were found to have bactericidal activity against stationary phase B. burgdorferi in the presently disclosed subject matter. Clofazimine was originally developed for the treatment of tuberculosis, although now it is commonly used for the treatment of leprosy (Arbiser and Moschella, 1995). The presently disclosed data showed that clofazimine was effective on the stationary phase B. burgdorferi, although the MIC of clofazimine is relatively high (6.25 μg/mL). Also, some sulfonamides, such as sulfameter and sulfisoxazole, were found to be effective against stationary phase B. burgdorferi, while sulfamethoxazole exhibited low values for MIC (≦0.2 μg/mL). These effective antibiotics may be regarded as candidates for further drug combination studies in animal models and clinical investigations.

In summary, a FDA-approved drug library consisting of about 2,000 compounds was screened on stationary phase B. burgdorferi enriched in non-replicating persisters using a newly developed SYBR Green I/propidium iodide (PI) assay. A number of drug candidates that have excellent activity against B. burgdorferi persisters were identified from existing drugs used for treating other diseases or conditions. These drugs include daptomycin, clofazimine, carbomycin, sulfa drugs like sulfamethoxazole and certain cephalosporins, such as cefoperazone. The presently disclosed subject matter provides methods that can be used to identify novel anti-persister activity against bacteria in the Borrelia genus.

### Example 2

### Drug Combinations Against Borrelia burgdorferi Persisters In Vitro: Eradication Achieved by Using Daptomycin, Cefoperazone and Doxycycline

### Materials and Methods

Strain, Media and Culture:

The strain, media, and culture were obtained and used as in Example I.

Antibiotics:

Doxycycline (Dox), amoxicillin (Amo), cefoperazone (CefP), clofazimine (Cfz), miconazole (Mcz), polymyxin B (Pmb), sulfamethoxazole (Smx), daptomycin (Dap), carbomycin (magnamycin A), vancomycin, nisin, carbencillin, ofloxacin, tigecycline, hydroxychloroquine, rifampin, and clarithromycin (Sigma-Aldrich) were dissolved in suitable solvents (Clinical and Laboratory Standards Institute, 2007) to obtain stock solutions. The antibiotic stocks were filter-sterilized by 0.2-μm filter except clofazimine, which was dissolved in DSMO (dimethylsulfoxide) and not filtered. Then the stocks were stored at −20° C.

Microscopy Techniques:

Specimens were examined on a Nikon Eclipse E800 microscope equipped with differential interference contrast (DIC) and epi-fluorescence illumination, and recorded with a Spot slider color camera. Cell proliferation assays were performed by direct counting using a bacterial counting chamber (Hausser Scientific Partnership) and DIC microscopy. SYBR Green I/PI assay was performed to assess the viability of B. burgdorferi. The ratio of live (green) and dead (red) B. burgdorferi was calculated by counting these cells using a bacterial counting chamber and epi-fluorescence microscopy. The three representative images of every sample were captured for quantitative analysis. Image Pro-Plus software was applied for measuring the biomass (fluorescence intensity) of different forms (spirochetes, round body, and microcolony) of B. burgdorferi as previously described (Shopov and Williams, 2000).

Antibiotic Exposure Assay:

To qualitatively determine the effect of antibiotics, 10 μL of each compound from the pre-diluted plate or pre-diluted stock was added to stationary phase B. burgdorferi culture in the 96-well plate. The final volume per well was adjusted to 100 μL at a concentration of 10 μg/mL for each antibiotic. Plates were sealed and placed in a 33° C. incubator for 7 days. The SYBR Green I/PI viability assay was used to assess the live and dead cells after antibiotic exposure as described (Feng, Wang, Shi, et al., 2014). Briefly, 10 μL of SYBR Green I (10,000×stock, Invitrogen) was mixed with 30 μL propidium iodide (PI, 20 mM) into 1.0 mL of sterile dH₂O. Then, 10 μL of staining mixture was added to each well and mixed thoroughly. The plates were incubated at room temperature in the dark for 15 minutes followed by plate reading at excitation wavelength at 485 nm and the fluorescence intensity at 535 nm (green emission) and 635 nm (red emission) in microplate reader (HTS 7000 plus Bio Assay Reader, PerkinElmer Inc., USA). With least-square fitting analysis, the regression equation and regression curve of the relationship between percentage of live bacteria and green/red fluorescence ratios was obtained. The regression equation was used to calculate the percentage of live cells in each well of the 96-well plate.

Subculture of Antibiotic-Treated B. burgdorferi to Assess Viability of the Organisms:

Seven-day-old B. burgdorferi culture (1×10⁷ spirochetes/mL) (500 μL) was treated with drugs or drug combinations in Eppendorf tubes. After incubation at 33° C. for 7 days without shaking, the cells were collected by centrifugation and rinsed with 1 mL fresh BSK-H medium followed by resuspension in 500 μL fresh BSK-H medium without antibiotics. Then 50 μL of cell suspension was transferred to 1 mL fresh BSK-H medium for subculture at 33° C. for 2 weeks. Cell proliferation was assessed using SYBR Green I/PI assay and bacterial counting chamber (Hausser Scientific Partnership) by microscopy as described above.

### Results

Taking advantage of a newly developed SYBR Green I/PI viability assay, an FDA-approved drug library was recently screened against stationary phase B. burgdorferi persisters and 27 drug candidates were identified that individually have higher activity than the currently recommended Lyme antibiotics doxycycline or amoxicillin (Example 1; Feng, Wang, Shi, et al., 2014). Among the top 27 confirmed drug candidates, daptomycin, clofazimine, carbomycin, sulfa drugs, such as sulfamethoxazole, and certain cephalosporins, such as cefoperazone, showed higher activity against B. burgdorferi persisters (Feng, Wang, Shi, et al., 2014). Interestingly, some drug candidates, such as daptomycin and clofazimine, with the highest activity against non-growing persisters had poor activity against actively growing B. burgdorferi with high MICs, at 12.5-25 μg/mL and 6.25 μg/mL, respectively (Feng, Wang, Shi, et al., 2014). Although these drug candidates active against persisters may not have good activity when used alone due to their poor activity against growing B. burgdorferi, it raises the question whether they may be used with another antibiotic, such as doxycycline, that is effective at inhibiting or killing the growing forms of B. burgdorferi. Such combinations may yield more effective treatment of Lyme disease.

Experimentally, since a stationary phase culture contains mixed populations of growing and non-growing bacteria that have different morphological variants, such as round bodies and microcolonies, that are tolerant to antibiotics (Feng, Wang, Shi, et al., 2014; Brorson et al., 2009; Sapi et al., 2011), it is most likely that a single drug may not effectively kill all bacterial populations including morphological variants. In the presently disclosed subject matter, a range of drug combinations was evaluated with the aim to identify optimal drug combinations that are most effective at killing B. burgdorferi persisters.

B. burgdorferi Culture Possesses Different Proportions of Morphological Variants Including Round Body and Microcolony Forms as the Culture Ages:

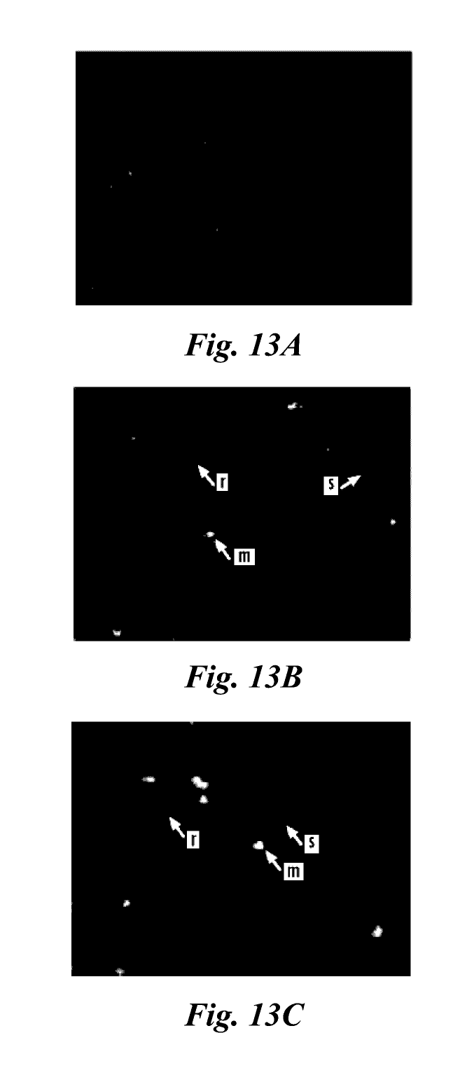

As shown in a previous study (Feng, Wang, Shi, et al., 2014), the stationary phase culture was enriched with morphological variants, such as round body form and biofilm-like aggregated microcolony, form in increasing proportions in contrast to individual spirochetes found in log phase culture (FIG. 13). To more accurately assess the proportion of different morphological variant forms, representative images of each sample taken from cultures of different ages were examined to measure the percentage of different morphological forms of B. burgdorferi (Table 2). It was found that the log phase (3-day-old) B. burgdorferi culture consisted almost entirely of spirochetal form (96%), with few round body form (4%) and no aggregated microcolony form (FIG. 13A). In the 7-day-old stationary phase culture of B. burgdorferi, there were 38% spirochetal form, 23% cystic or round body form, and 39% microcolony form (FIG. 13B). When B. burgdorferi stationary phase culture was cultured for 10 days, the percentage of the microcolony form increased to 64%, and the spirochetal form and the round body form were 20% and 16%, respectively (FIG. 13C).

Persister Frequencies in Log Phase and Stationary Phase Cultures:

Because B. burgdorferi does not form colonies easily on agar plates, the conventional method to assay persister frequency after antibiotic exposure by calculating the percentage of bacteria killed by a bacteriocidal antibiotic cannot be applied to B. burgdorferi. Therefore, the frequency of B. burgdorferi persisters in log phase and stationary phase cultures was assessed using the SYBR Green I/PI viability assay after exposure of the cultures to antimicrobials. E. coli culture was used as a control after exposure to antibiotics to validate the SYBR Green I/PI viability assay for persister frequency assessment. The persister frequency of the log phase E. coli culture with exposure to 50 μg/mL amoxicillin for 3 hours was 4.4% for the SYBR Green I/PI assay and 0.9% for the CFU assay (Table 1). Using the SYBR Green I/PI assay, the persister frequencies of B. burgdorferi ranged from 5-10% for log phase cultures, but ranged 16-27% in stationary phase cultures treated with ceftriaxone, doxycycline or amoxicillin (Table 1). Given that the SYBR Green I/PI viability assay seemed to give about 5 fold (4.4%/0.9%) overestimation of the persister frequency over the CFU assay with the E. coli control, the real persister frequencies of B. burgdorferi are likely to be in the range of 1-2% for B. burgdorferi log phase cultures and 3-5.5% for stationary phase cultures.

Microcolony Form is More Tolerant to Antibiotics than Free-Living Spirochetal and Round Body Forms:

Previous studies showed that the stationary phase B. burgdorferi was more resistant or tolerant to antibiotics than the log phase culture (Feng, Wang, Shi, et al., 2014). In view of the heterogeneity of the morphological variants of the stationary phase culture (FIG. 13B and FIG. 13C), the susceptibility of different variant forms of B. burgdorferi to commonly employed antibiotics for Lyme disease (doxycycline, amoxicillin, and ceftriaxone) was determined in a more quantitative manner. Interestingly, it_{was} found that different variant forms had differing susceptibilities to these antibiotics (Table 2). The log phase culture (3-day-old) primarily consisting of spirochetal form was highly susceptible to these antibiotics, whereas the stationary phase (7- and 10-day old) cultures comprising mainly of round body and biofilm-like microcolony forms were less sensitive to these antibiotics, as shown by increasing proportion of viable cells remaining after the antibiotic exposure (Table 2).

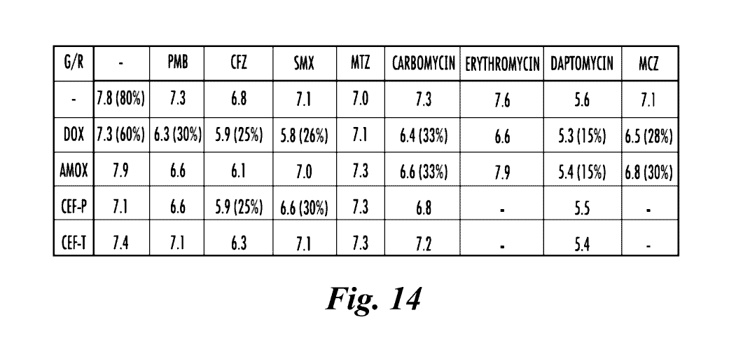

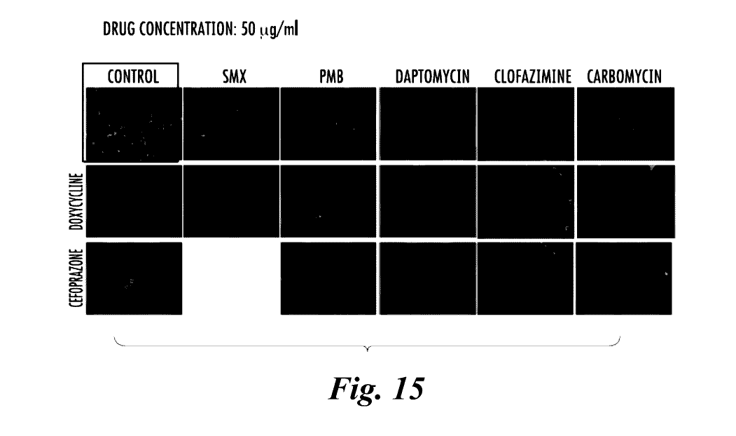

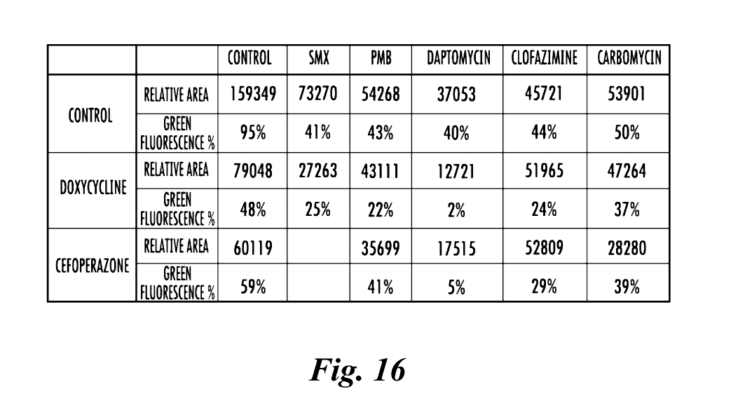

FIG. 14 shows the effect of drugs (50 μg/mL) and combinations on stationary phase Borrelia. FIG. 15 shows some promising drug combinations against Borrelia biofilm. FIG. 16 shows the activity of drug combinations against Borrelia biofilm.

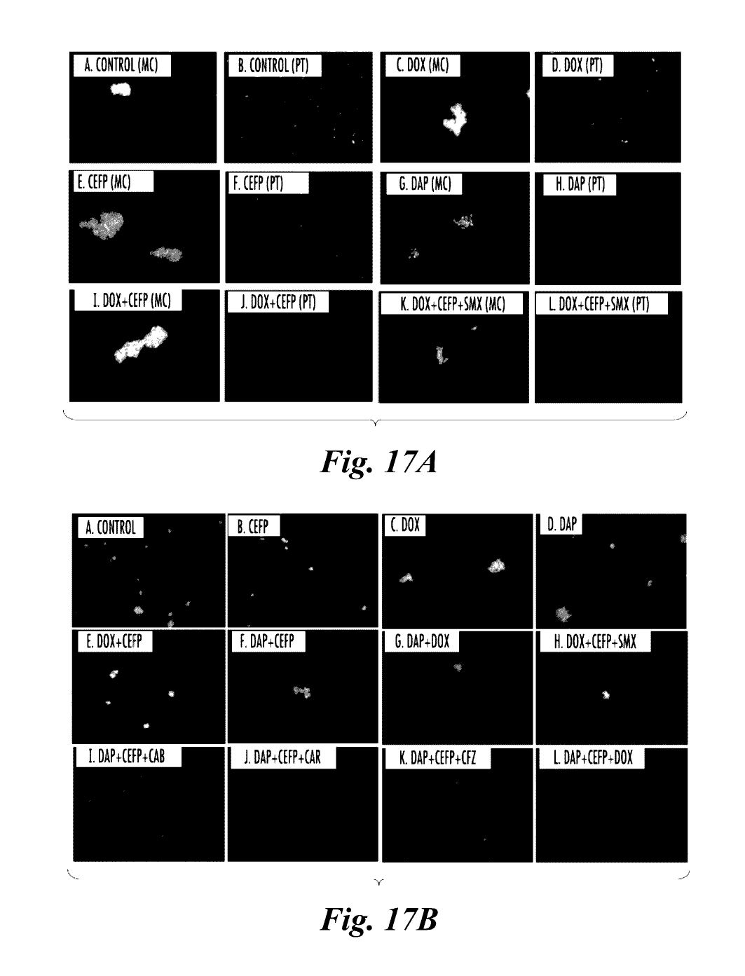

When the 10-day-old stationary phase culture, consisting of mixed populations of spirochetal form in minor portions and round body form and microcolony form in major proportions, was exposed to various antibiotics, it was found that the microcolony form was more tolerant to antibiotics than the free-living spirochetal form and the round body form. Daptomycin at 10 μg/mL, a drug with high activity against B. burgdorferi persisters (Feng, Wang, Shi, et al., 2014), killed all planktonic forms (spirochetal and round body) of stationary phase cells (FIG. 17A, Panel h), but could only partially kill the microcolony form of B. burgdorferi persisters as shown by the presence of significant numbers of red cells (dead cells) mixed with some green cells (viable cells) in the microcolony (FIG. 17A, Panel h). The other persister active drug cefoperazone (Feng, Wang, Shi, et al., 2014) had weaker activity than daptomycin since it had some activity for the planktonic form cells (52% cells were green cells), but little activity for the microcolony form of persisters where most of the microcolony cells remained as green (live) cells (FIG. 17A, Panel e). In contrast, doxycycline had the least activity against stationary phase B. burgdorferi persisters where about 71% free-living planktonic cells including spirochetal form and round body form were not killed by doxycycline as shown by green (live) cells (FIG. 17A, Panels d, f and j), but the microcolony form was almost all live (FIG. 17A, Panels c, e and i). These findings suggest there is a differential tolerance or resistance in different variant forms of persisters in vitro (spirochetal form, round body form and microcolony in increasing order of resistance) to both current Lyme disease antibiotics and also even persister active antibiotics daptomycin and cefoperazone, with the microcolony form being the most tolerant to antibiotics.

Effect of Drug Combinations on Stationary Phase B. burgdorferi Persisters:

Despite the powerful anti-persister activity of daptomycin and cefoperazone, they had limited activity to kill the most resistant microcolony form of persisters at 10 μg/mL (FIG. 17). These findings suggest that these FDA-approved persister drugs may have limited potential if used alone against B. burgdorferi. To identify more effective drug combinations that kill different variant forms of B. burgdorferi stationary phase persisters, 81 drug combinations were evaluated including FDA-approved drugs on a 10-day-old B. burgdorferi culture enriched with microcolony and round body forms at 10 μg/mL of each individual drug (close to or lower than MIC). The results showed that some drug combinations were indeed much more effective than single drugs alone (Table 2). Among them, daptomycin highlighted itself as having the best activity against stationary phase B. burgdorferi persisters when combined with other drugs.

Daptomycin (10 μg/mL) alone could not eliminate the microcolonies by itself (FIG. 17A, Panel g), but daptomycin in combination with doxycycline or beta-lactams was very effective against B. burgdorferi planktonic persisters and also against microcolonies (Table 2, FIG. 17B). However, daptomycin in combination with doxycycline or cefoperazone produced better bacteriocidal activity for the microcolony form than either of these agents alone or drug combinations without daptomycin, such as doxycycline+cefoperazone or even doxycycline+cefoperazone+sulfamethoxazole, as shown by more red cells (dead cells) being produced after daptomycin drug combinations (FIG. 17B, Panels f, g, i, j, k and l). Nevertheless, daptomycin used as part of two drug combinations did not completely eradicate microcolony form of persisters (FIG. 17B, Panels f and g). Remarkably, daptomycin as part of a three drug combination using doxycycline and cefoperazone eradicated all microcolonies with only few traces of red (dead) cells left (FIG. 17B, Panels h, i, j, k and l), whereas other daptomycin-containing three drug combinations using cefoperazone+ either carbenicillin or carbomycin or clofazimine still had some red microcolonies remaining after treatment.

In addition to doxycycline and beta-lactams, some clinical drugs, such as vancomycin, ofloxacin, clarithromycin, and hydroxychloroquine, which are not recommended for treating Lyme disease, also exhibited some weak activity on the 10-day-old stationary phase B. burgdorferi culture, either alone or in combination with doxycycline and cefoperazone. Rifampin alone did not have significant activity for B. burgdorferi persisters, but in combination with doxycycline, amoxicillin, ceftriaxone or cefoperazone had higher activity for B. burgdorferi persisters (Table 3). Among all the other non-daptomycin drug combinations, the only two drug combinations that are close to daptomycin drug combinations in killing B. burgdorferi persisters were Dox+ either CefP or miconazole or sulfamethoxazole (Table 3). In addition, clofazimine showed good activity against stationary phase B. burgdorferi persisters when combined with doxycycline and cefoperazone (Table 3). It is worth noting that the activity of carbenicillin, vancomycin, ofloxacin, clarithromycin, tigecycline, nisin, and hydroxychloroquine when combined with doxycycline only marginally enhanced doxycycline activity and their anti-persister activities were not as effective as when they were combined with daptomycin (Table 3).

Subculture of Antibiotic-Treated B. burgdorferi:

In a previous study, it was found that daptomycin at 50 μM (equivalent to 81 μg/mL, a high dose to achieve in humans) had remarkable anti-persister activity that seemed to kill all B. burgdorferi persisters, as shown by all red cells stained by PI (FIG. 3D in Feng, Wang, Shi, et al., 2014). To confirm that these red cells are indeed dead, a subculture test in fresh BSK-H medium was performed and it was found that indeed these red cells treated with 50 M daptomycin were dead as they failed to grow in the subculture test as shown by lack of any visible green spirochetes after 15 day subculture (data not shown). Having established the subculture test as a reliable assay for assessing the viability of antibiotic treated cells, the above results obtained with select antibiotics or antibiotic combinations that produced the best bacteriocidal effects against persisters were validated (see FIG. 17).

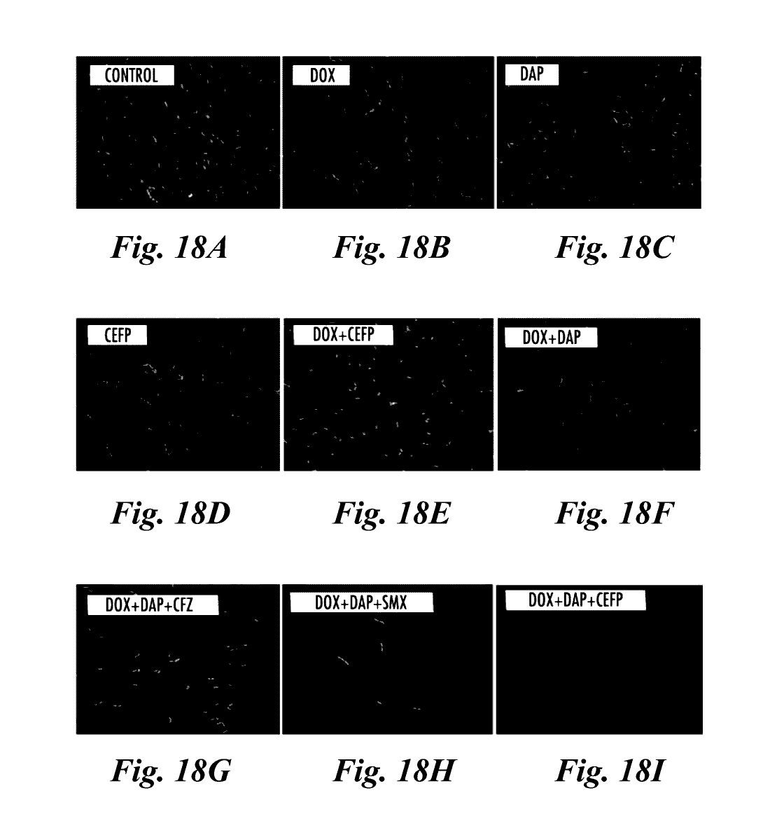

To do this, a 7-day-old stationary phase B. burgdorferi culture was subjected to exposure with select antibiotics and antibiotic combinations for 7 days, followed by subculture in fresh BSK-H medium for 7 days or 15 days. Microscope counting showed that drug-free controls and samples treated with single drug grew in the 7-day subculture. Samples treated with two drug combinations grew more slowly (Table 4). However, after the 7-day subculture, all the three drug combinations, e.g., doxycycline+daptomycin+ either cefoperazone or Smx or Cfz did not show any sign of growth as no visible spirochetal form was observed, whereas other drug combinations all had visible green spirochetes under the microscope. After the 15-day subculture, there were about 1×10⁷ spirochetes in the control sample and 5×10⁶ spirochetes in doxycycline or amoxicillin treated samples (Table 4). Interestingly, daptomycin alone, or two drug combinations doxycycline+cefoperazone and doxycycline+daptomycin, or even three drug combination doxycycline+daptomycin+clofazimine, could not sterilize the B. burgdorferi persisters, as they all had visible spirochetes growing after subculture (FIG. 18).

However, doxycycline+daptomycin+sulfamethoxazole significantly reduced the number of spirochetes with very few spirochetes being visible after the 15-day subculture (FIG. 18h). By far the best result was achieved with daptomycin in combination with doxycycline and cefoperazone, which killed all B. burgdorferi persisters with no viable bacteria observed (FIG. 18i). This is demonstrated by a decrease in the Green/Red fluorescence and lack of any viable green spirochetes, in contrast to samples treated with other drugs alone or drug combinations, which all had higher Green/Red fluorescence and visible green spirochetal bacteria (Table 4, FIG. 18). Importantly, this drug combination could eliminate not only planktonic stationary phase B. burgdorferi persisters (spirochetal and round body forms), but also the more resistant biofilm-like microcolonies (Table 4, FIG. 18). Subculturing the sample treated with this drug combination showed no sign of any detectable organisms by microscopy (detection limit<10⁵) even after 15 days of subculture (Table 4, FIG. 18i). These findings indicate that the microcolony structures are not eliminated by doxycycline, amoxicillin, persister active drugs alone, two drug combinations or even some three drug combinations, but could be eradicated by the drug combination of doxycycline, cefoperazone and daptomycin.

TABLE 2
Varying degrees of susceptibility of different forms of B. burgdorferi to commonly used Lyme antibiotics

| | Percentage of different forms of B. burgdorferi ^{a} | | | | | | | | | | |
|---|---|---|---|---|---|---|---|---|---|---|---|
| | | Cystic or | | | | | | | | E. coli control^{e} | |
| | | round | | Percentage of residual viable cells^{b,c} | | | | | | Persister | Persister |
| | Spirochete | body form | Microcolony | Doxycycline | | Amoxicillin | | Ceftriaxone | | frequency^{f} | frequency^{g} |
| 3-day log phase | 96% | 4% | 0% | 8% | (6.4%) | 23% | (9.6%) | 6% | (5.8%) | 4.4% | 0.9% |
| culture^{d} | | | | | | | | | | | |
| 7-day stationary | 38% | 23% | 39% | 71% | (24%) | 80% | (25%) | 47% | (16%) | — | — |
| phase culture | | | | | | | | | | | |
| 10-day stationary | 20% | 16% | 64% | 80% | (~25%) | 83% | (~27%) | 70% | (~25%) | — | — |
| phase culture | | | | | | | | | | | |

^{a} Percentages of different forms of B. burgdorferi were calculated by measuring three representative microscope images with Image Pro-Plus software.
^{b}Percentages of residual viable B. burgdorferi relative to drug-free control after drug treatment were calculated according to the regression equation and ratio of Green/Red fluorescence obtained by SYBR Green I/PI assay as described (Feng, Wang, Shi, et al., 2014). The samples were treated with antibiotics for 7 days before viability was assessed by the SYBR Green I/PI assay.
^{c}Values in brackets indicate persister frequencies (percentage of live cells after antibiotic treatment). The number of B. burgdorferi assayed by epi-fluorescence microscope counting was calibrated using E. coli as a control.
^{d}The log phase culture was obtained by subculture of a stationary phase culture at 1:50 dilution for 3 days in BSK medium.
^{e}Three hour log phase E. coli culture (1.7 × 10⁸ cfu/mL) was treated with 50 μg/mL amoxicillin for 3 hours followed by persister frequency determination.
^{f}Persister frequency calculated by epi-fluorescence microscope counting after SYBR Green I/PI viability staining.
^{g}Persister frequency calculated by standard plate colony count assay.

TABLE 3
Effect of drug combinations on stationary phase B. burgdorferi culture^{a}

| Live Cell % | C | Mcz | Pmb | Smx | Dap | Cfz^{b} | Car | Van | Cab | Ofl | Clar | Tig | Hcq | Rif^{b} |
|---|---|---|---|---|---|---|---|---|---|---|---|---|---|---|
| C | 87% | 65% | 77% ^{c} | 82% ^{c} | 52% | 73% | 65% | 64% | 67% | 81% ^{c} | 63% | 67% | 71% | 82% ^{c} |
| Dox | 72% | 53% | 64% | 71% | 32% | 43% | 59% | 63% | 64% | 60% | 60% | 59% | 68% | 60% |
| Amo | 75% ^{c} | 51% | 75% | 68% | 41% | 56% | 63% | — | — | — | — | — | — | 60% |
| Ceftriaxone | 68% | 48% | 64% | 64% | 41% | 57% | 63% | — | — | — | — | — | — | 59% |
| Cefoperazone | 64% | 45% | 64% | 65% | 41% | 46% | 60% | 64% | 63% | 62% | 63% | 51% | 53% | 58% |
| Dox + CefP | 59% | 39% | 59% | 37% | 19% | 43% | 55% | 50% | 59% | 56% | 55% | 51% | 48% | — |
| Dap | 48% | 37% | 35% | 27% | 35% | 20% | 26% | 23% | 20% | 27% | 32% | 23% | 31% | — |
| Dap + Dox | 34% | 34% | 33% | 24% | 32% | 20% | 23% | 25% | 16% | 20% | 31% | 21% | 25% | — |

^{a}Ten-day-old stationary phase B. burgdorferi culture enriched with micro-colonies was treated with 10 μg/mL drugs alone or in different combinations for 7 days. The percentage of residual viable B. burgdorferi was calculated according to the regression equation and ratio of Green/Red fluorescence using the SYBR Green I/PI assay as described (Feng, Wang, Shi, et al., 2014). Direct microscopy counting was employed to verify the results of SYBR Green I/PI assay. The most effective drug combinations as reflected by residual viable cell percentages of less than 30% are shown in bold type. The best drug combinations without daptomycin are underlined. Abbreviations: Dox, doxycycline; Amo, amoxicillin; CefP, cefoperazone; Cfz, clofazimine; Mcz, miconazole; Pmb, polymyxin B; Dap, daptomycin; Smx, sulfamethoxazole; Cab, carbencillin; Car, carbomycin; Van, vancomycin; Ofl, ofloxacin; Clar, clarithromycin; Tig, tigecycline; Hcq, hydroxychloroquine; Rif, rifampin. “—” indicates not determined. C = drug-free control
^{b}To eliminate the influence of red color of antibiotics, fluorescence data was corrected using antibiotic control.
^{c} P-values of the standard t-test for the all treated group versus the drug-free control were less than 0.01 except the data marked with “c”.

TABLE 4
Subculture tests to assess the viability of drug-treated stationary phase B. burgdorferi.^{a}

| | Residual | G/R ratio | | | | |
|---|---|---|---|---|---|---|
| | viable | immediately | After 7 day subculture | | After 15 day subculture | |
| Drugs^{b} | cells^{c} | after treatment^{d} | G/R ratio^{d} | Spirochetes^{e} | G/R ratio^{d} | Spirochetes^{e} |
| Control | 82% | 7.32 | 7.28 | 5 × 10⁶ | 7.38 | 6 × 10⁶ |
| Dox | 67% | 6.71 | 6.23 | 9 × 10⁵ | 6.89 | 4 × 10⁶ |
| Amoxicillin | 80% | 7.16 | 6.32 | 1 × 10⁶ | 7.23 | 6 × 10⁶ |
| Dox + Dap + CefP | 10% | 5.32 | 4.93 | <1 × 10⁵ | 4.82 | <1 × 10⁵ |
| Dox + Dap + Cfz | 15% | 4.96 | 4.86 | <1 × 10⁵ | 7.38 | 6 × 10⁶ |
| Dox + Dap + Smx | 18% | 5.85 | 5.34 | <1 × 10⁵ | 6.41 | 2 × 10⁶ |
| Dox + Dap | 23% | 6.03 | 5.91 | 3 × 10⁵ | 7.31 | 6 × 10⁶ |
| Dox + CefP | 56% | 6.35 | 6.01 | 7.5 × 10⁵ | 6.87 | 4 × 10⁶ |
| Dap | 55% | 5.85 | 7.01 | 3.1 × 10⁶ | 7.11 | 5 × 10⁶ |
| CefP | 61% | 6.60 | 6.13 | 1.5 × 10⁶ | 6.95 | 5 × 10⁶ |

^{a}Seven-day-old B. burgdorferi culture (1 × 10⁷ spirochetes/mL) (500 μL) was treated with 10 μg/mL drugs alone or drug combinations for 7 days. Then, 50 μL of washed bacterial cells was subcultured in 1 mL fresh BSK-H medium for 7 days and 15 days.
^{b}Abbreviations: G/R ratio, Green/Red fluorescence ratio; Dox, doxycycline; CefP, cefoperazone; Cfz, clofazimine; Dap, daptomycin; Smx, sulfamethoxazole.
^{c}Residual viable B. burgdorferi was assayed by epifluorescence microscope counting.
^{d}Green/Red fluorescence ratios were obtained by microplate reader after SYBR Green I/PI staining. Each value is the mean of three replicates.
^{e}The number of spirochetes was evaluated by microscope counting.

### Discussion

In the presently disclosed subject matter, the first in vitro drug combination study using persister active drugs was conducted (Feng, Wang, Shi, et al., 2014) in combination with the currently recommended Lyme antibiotics, such as doxycycline or amoxicillin or other antibiotics, to achieve more effective eradication of B. burgdorferi persisters. It was found that it is more effective to kill B. burgdorferi persisters by drug combination than single antibiotic, but bacteriocidal activity depended on the particular antibiotics used (Table 3). It is interesting to note that despite the persister active antibiotics, such as the lipopeptide daptomycin and beta-lactam cefoperazone themselves, were quite active against planktonic B. burgdorferi persisters (both spirochetal and round body forms), they were unable to eradicate the more resistant microcolony form when used alone or even in combination (FIG. 17). Previous studies showed that tinadazole, metronidazole, and tigecycline were more active against B. burgdorferi round body and microcolonies than doxycycline and amoxicillin, but they could not completely kill the microcolonies even at high concentrations of antibiotics (Sapi et al., 2011), indicating the limited activity of these antibiotics used singly against B. burgdorferi persisters. Although tigecycline was the most active antibiotic against the round body form compared with tinadazole and metronidazole in that study (Sapi et al., 2011), it was found that by itself tigecycline was not very effective at killing the biofilm-like microcolonies (Table 3).

Remarkably, it was found that daptomycin in combination with doxycycline and cefoperazone or carbencillin was able to completely eradicate the most resistant microcolonies (FIG. 17), and this was further confirmed by subculture studies, which showed lack of any growth (FIG. 18). While various drug combinations showed improved activity against stationary phase B. burgdorferi persisters, daptomycin combinations had the best activity among drug combinations against persisters (Table 3). The only non-daptomycin regimens that were close to daptomycin combinations contained cefoperazone (FIG. 17, Table 3). Unexpectedly, other antibiotics, such as sulfamethoxazole, clofazimine and miconazole, also had more activity against stationary phase B. burgdorferi persisters in combination with doxycycline and cefoperazone. These drugs are not currently used as antibiotics for treatment of Lyme disease clinically (CDC, Post-Treatment Lyme Disease Syndrome, 2014; Hunfeld and Brade, 2006). Although sulfa drugs are bacteriostatic when used alone for growing bacteria, they could kill non-growing round body or aggregated microcolony form of B. burgdorferi during long-term treatment. Clofazimine with high anti-persister activity improved the combination with daptomycin or daptomycin plus doxycycline (Table 3), which may be due to its multiple mechanisms of action including membrane destabilization, reactive oxygen species production, and inhibition of membrane energy metabolism in M. tuberculosis (Xu et al., 2012). It also was found that miconazole, an imidazole antifungal drug, had high activity against B. burgdorferi persisters when combined with doxycycline and cefoperazone (Table 2). Miconazole has been shown to alter the integrity of lipid membrane (Vanden Bossche et al., 1989) and therefore may facilitate the penetration of other drugs, such as doxycycline and cefoperazone, for improved activity against B. burgdorferi persisters (Table 3).

The complete eradication of the B. burgdorferi biofilm-like microcolonies by the three drug combination of daptomycin+doxycycline+cefoperazone has not been achieved with any single, dual or even some three drug combinations in the presently disclosed subject matter or any other previous studies. The mechanism by which this three drug combination was able to achieve this remarkable activity is worth commenting. Without wishing to be bound to any one particular theory, doxycycline and cefoperazone inhibits protein synthesis and cell wall peptidoglycan synthesis respectively (Kersten et al., 1995). Either may be needed to kill the growing forms present in the B. burgdorferi microcolonies or those occasionally revert to growing forms from microcolonies, but these drugs are less effective against the round body or microcolony persisters themselves (Feng, Wang, Shi, et al., 2014; Brorson et al., 2009; Sapi et al., 2011). This inability could be because of the reduced drug penetration into the microcolony structure, efflux mechanism (Brorson et al., 2009; Casjens, 2000), or decreased protein or cell wall synthesis in persisters. The high efficacy of daptomycin against B. burgdorferi persisters could be due to its effect on membrane disruption or depolarization, resulting in a loss of membrane potential and inhibition of energy metabolism (Feng, Wang, Shi, et al., 2014; Pogliano et al., 2012), which is required for persister survival (Zhang, 2014). Prior studies have suggested that the combination of beta-lactams plus daptomycin increase effectiveness even with daptomycin resistant Gram-positive infections (Dhand et al., 2011). While drugs traditionally active against Gram-positive organisms are not thought to have activity against B. burgdorferi, in vitro studies have previously documented activity with drugs, such as vancomycin (Hall et al., 2014; Dever et al., 1993), but not teicoplanin or daptomycin, though this study was performed examining not persisters but log phase cultures. Though daptomycin is not used for Gram-negative pathogens, a drug, such as colistin, has been suggested to improve polyanionic lipopeptide activity due to outer membrane permeabilization (Morris et al., 1995). Regardless, these studies suggest that combined use of these agents that kill or inhibit both growing organisms (doxycycline and cefoperazone) and non-replicating organisms (daptomycin and cefoperazone) are important for good activity against the highly resistant microcolonies, which is consistent with the proposition to use drugs targeting both growing and non-growing microbial populations for improved treatment of persistent infections (Zhang, 2014).

It is worth noting that short term incubation in subculture studies of antibiotic treated B. burgdorferi is not sufficient to assess the stable eradication of persisters. This is shown by the 7-day subculture of B. burgdorferi persister cells treated with three drug combinations daptomycin+doxycycline+cefoperazone or Smx or Cfz, which all produced no detectable levels of any residual growth (Table 4). However, extended incubation to 15 days of subculture showed that only daptomycin, doxycycline and cefoperazone combination was able to completely eradicate biofilm-like microcolonies with no detectable spirochetes (FIG. 18i). These findings suggest that longer incubation to 15 days or more in post-antibiotic exposure may be needed to thoroughly assess the drug combinations that stably eradicate the persister forms without relapse. The subculture results do validate the SYBR Green I/PI viability assay and is a useful and more sensitive technique to assess the viability of B. burgdorferi persisters or microcolonies after drug treatment in identifying optimal drug combinations for killing more resistant persisters.

B. burgdorferi spirochetes could develop morphological variants as in vitro cultures age or are subjected to adverse conditions (Feng, Wang, Shi, et al., 2014; Brorson et al., 2009; Alban et al., 2000; Sapi et al., 2011; Murgia and Cinco, 2004). The proportions of these variants have not been well characterized over time in culture conditions. With careful measurement, the percentages of morphological variants were determined as they transitioned from spirochetes to progressively round body form to then microcolony form as log phase culture grew to stationary phase (7-10 days) (FIG. 13). Although previous studies reported the round body form or biofilm-like microcolony form is more resistant to antibiotics (Feng, Wang, Shi, et al., 2014; Brorson et al., 2009; Sapi et al., 2011), their relative resistance was not fully studied. Here, a hierarchy or varying levels of stationary phase B. burgdorferi persisters have been found in terms of their levels of persistence as the morphology of the variants changes from spirochetes, to round body, and to microcolony forms, with increasing antibiotic tolerance (Table 2).

The finding that persister frequencies are higher in stationary phase B. burgdorferi cultures than in log phase cultures is consistent with studies in other bacteria. However, the persister frequencies in B. burgdorferi log phase culture (5-10%) and stationary phase cultures (16-27%) determined by SYBR Green I/PI assay seem to be higher than those reported for E. coli (0.001% in log phase to 1% in stationary phase) (Keren et al., 2004). Given that the SYBR Green I/PI assay tended to overestimate the persister frequency by about 5 fold based on the E. coli data (4.4%/0.9%) (Table 2), the converted persister frequencies of 1-2% and 3-5% for B. burgdorferi log phase and stationary phase cultures would still suggest higher persister frequencies with B. burgdorferi. This could reflect differences in their ability to form persisters, the speed of growth of the organisms, the age of culture when antibiotic is added, and the dilution factor, which affects the number of persisters carried over during the subculture. In addition, it has been found that the persister frequencies vary according to the antibiotic used, with the more effective antibiotic ceftriaxone having a lower persister frequency than amoxicillin (Table 2), a finding that is consistent with previous studies (Zhang, 2014; Lu and Zhang, 2007). It remains to be determined if there are differences in persistence of B. burgdorferi strains and if the high persister frequencies in B. burgdorferi strains are associated with recalcitrance to antibiotic therapies.

In summary, it has been found that there is a hierarchy of in vitro B. burgdorferi persisters with increasing antibiotic tolerance as the culture ages from log phase to stationary phase with morphological changes from spirochetal form to round body and microcolony forms. Persister frequencies in log phase B. burgdorferi culture ranged 5.8-9.6% depending on the antibiotic as measured by SYBR Green I/propidium iodide (PI) viability stain and microscope counting, but the corrected log phase B. burgdorferi persister frequencies were at 1-2% using E. coli as a control. To more effectively eradicate these persister forms tolerant to doxycycline or amoxicillin, drug combinations were studied using previously identified drugs from an FDA-approved drug library with high activity against B. burgdorferi persisters. Using a SYBR Green/PI viability assay, daptomycin-containing drug combinations were the most effective at killing B. burgdorferi persisters. Of studied combinations, daptomycin was the common element in the most active regimens against persisters when used with doxycycline plus either beta-lactams (cefoperazone or carbenicillin) or energy inhibitor (clofazimine). Daptomycin plus doxycycline and cefoperazone eradicated the most resistant microcolony form of B. burgdorferi persisters and did not yield viable spirochetes upon subculturing, suggesting durable killing of these persisting forms, which was not achieved by any other two or three drug combinations. These findings may have implications for treatment of Lyme disease patients with stubborn ongoing symptoms or antibiotic-refractory arthritis, if persistent organisms or detritus are responsible for symptoms that do not resolve with conventional therapy.

### Example 3

### FDA-Approved Drugs Active Against the Round Body Form of Borrelia burgdorferi Persisters

### Materials and Methods

Strain, Media and Culture:

B. burgdorferi strain B31 was obtained from American Type Tissue Collection. B. burgdorferi and was cultured in BSK-H media (HiMedia Laboratories Pvt. Ltd.), with 6% rabbit serum (Sigma-Aldrich, Co). All culture media were filter-sterilized by 0.2 μM filter. Cultures were incubated in sterile 50 mL closed conical tubes (BD Biosciences, California, USA) at 33° C. without antibiotics.

Induction of Round Body Form of B. burgdorferi:

For induction of round body form of B. burgdorferi, B. burgdorferi spirochetes (1×10⁵ spirochetes/mL) were cultured in BKS-H medium for 6 days without shaking. After the 6-day incubation, amoxicillin at a final concentration of 50 μg/mL was added to the culture for round body form induction. After 72 h induction at 33° C., the round body forms of B. burgdorferi were examined by the microscopy. The round body cells (100 μL) were transferred to 96-well tissue culture microplates for evaluation of the effects of antibiotic treatment.

Microscopy Techniques:

Specimens were examined on a Nikon Eclipse E800 microscope equipped with differential interference contrast (DIC) and epi-fluorescence illumination, and recorded with a Spot slider color camera. Cell proliferation assays were performed by direct counting using a bacterial counting chamber (Hausser Scientific Partnership, PA, USA) and DIC microscopy. To assay the viability of B. burgdorferi, the SYBR Green I/PI assay (Feng, Wang, Shi, et al., 2014) was performed. The ratio of live (green) and dead (red) B. burgdorferi was calculated by counting the cells using a bacterial counting chamber and epi-fluorescence microscopy.

Antibiotics and FDA Drug Library:

Doxycycline, metronidazole, cefmetazole, roliteracycline, sulfachlorpyridazine, artemisinin, cefoperazone, daptomycin (Sigma-Aldrich) were dissolved in suitable solvents (Wikler and Ferraro, 2008) to form stock solutions. The antibiotic stocks were filter-sterilized by 0.2 μm filter. Then the stocks were pre-diluted into 500 μM pre-diluted stocks and stored at −20° C.

Each drug in the JHCCL FDA-approved drug library (Ricker et al., 2011) was made to 10 mM stock solutions with DMSO. The stock solutions were arrayed in a total of 24 96-well plates, leaving the first and the last columns in each plate as controls. Each solution in these master plates was diluted with PBS to make 500 μM pre-diluted working stock plates. The first and the last columns in each pre-diluted plate were set as blank controls, doxycycline control, and amoxicillin control. The pre-diluted drug stock plates were sealed and stored at −20° C.

Antibiotic Susceptibility Test:

To qualitatively determine the effect of antibiotics, 10 μL of each compound (final concentration 50 μM) from the pre-diluted plate or pre-diluted stock was added to round body form or stationary phase B. burgdorferi culture in the 96-well plate. The final volume per well was adjusted to 100 μL. Plates were sealed and placed in a 33° C. incubator for 7 days. The SYBR Green I/PI viability assay was used to assess the live and dead cells after antibiotic exposure as described (Feng, Wang, Shi, et al., 2014). Briefly, 10 μL of SYBR Green I (10,000×stock, Invitrogen) was mixed with 30 μL propidium iodide (PI, 20 mM, Sigma-Aldrich) into 1.0 mL of sterile dH₂O. Then, 10 μL staining mixture was added to each well and mixed thoroughly. The plates were incubated at room temperature in the dark for 15 minutes followed by plate reading at excitation wavelength at 485 nm and the fluorescence intensity at 535 nm (green emission) and 635 nm (red emission) in microplate reader (HTS 7000 plus Bio Assay Reader, PerkinElmer Inc., USA). With least-square fitting analysis, the regression equation and regression curve of the relationship between percentage of live and dead bacteria as shown in green/red fluorescence ratios was obtained. The regression equation was used to calculate the percentage of live cells in each well of the 96-well plate.

### Results

Induction of Round Body Form of B. burgdorferi by Amoxicillin:

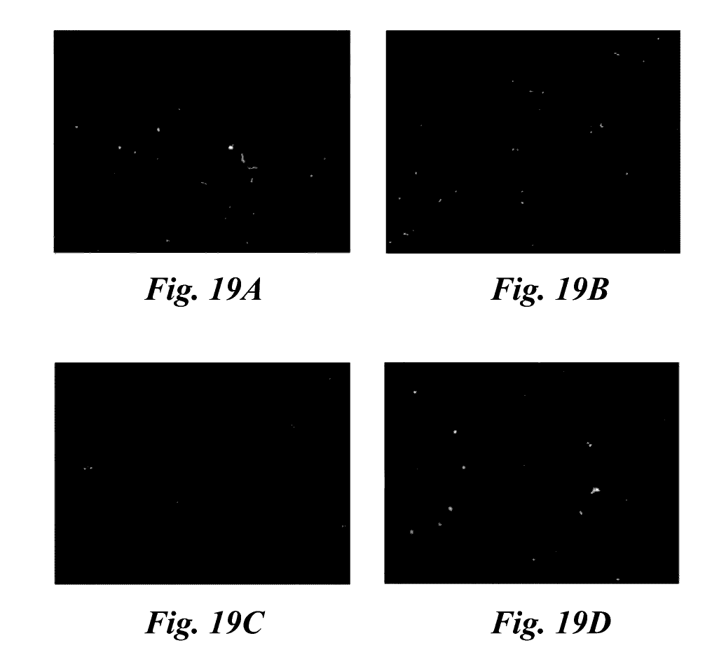

Beta-lactam antibiotics are the most commonly used frontline drugs for the treatment of Lyme disease, but intriguingly could induce spirochetal B. burgdorferi to form round bodies which are resistant to Lyme antibiotics (Brorson et al., 2009; Sapi et al., 2011). In order to identify FDA-approved drugs active against the round body form of B. burgdorferi, the optimal conditions for induction of round body form were assessed. It was found that 6-day or older culture could not be induced to round body form completely with even 100 μg/mL amoxicillin (FIG. 19D). It was found that the best condition for producing the round body for use in a FDA drug library screen was 5-day B. burgdorferi culture treated with 50 μg/mL amoxicillin for 72 h. Microscopic examination showed that under the above inducing condition, up to about 96% of the B. burgdorferi spirochetes could be induced into round body form by amoxicillin (FIG. 19A). To confirm that the induced round body form was still viable, a subculture test in fresh BSK-H medium was performed. The round bodies (in 500 μL culture) were collected by centrifugation and rinsed with 1 mL fresh BSK-H medium followed by resuspension in 500 μL fresh BSK-H medium. Then, 50 μL of cell suspension was transferred to 1 mL fresh BSK-H medium for subculture at 33° C. for 5 days. Microscopy analysis revealed that the amoxicillin-induced round body form of B. burgdorferi could revert to spirochetes (up to 95%) in BSK-H medium after the 5-day subculture (FIG. 19), indicating that the round body form induced by and tolerant to amoxicillin treatment is fully viable.

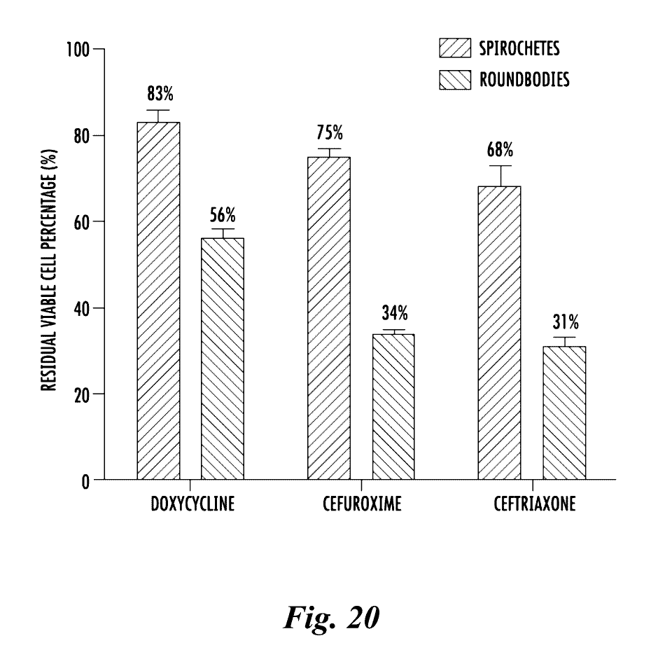

To compare the antibiotic susceptibility of the round body form of B. burgdorferi with the spirochetal form, commonly used Lyme disease antibiotics doxycycline, cefuroxime, and ceftriaxone were tested on 5-day old spirochetes and the amoxicillin induced round body form of B. burgdorferi. The results showed that the round body form of B. burgdorferi was more tolerant or resistant to antibiotics than the spirochetal form (FIG. 20). The amoxicillin induced round body form was subsequently used for drug screens as described below.

Screen for Effective Drugs Against the Round Body Form of B. burgdorferi:

In a previous study, a SYBR Green I/PI assay was developed which can be used as a high-throughput screening method for rapid viability assessment for B. burgdorferi (Feng, Wang, Shi, et al., 2014). In this study, this rapid SYBR Green I/PI assay was used to identify drugs which have activity against the round body form of B. burgdorferi persisters by using an FDA-approved drug library. Since metronidazole was shown to kill the round body form of B. burgdorferi (Amant et al., 2012), metronidazole and doxycycline were included as control drugs in the screen. In the initial screen, the effective hits were selected as having residual viable cell ratios below that of the amoxicillin control. Hit compounds were selected for further rescreens, followed by microscope counting to verify the screening results. Epi-fluorescence microscope counting further validated the effective drug candidates by the SYBR Green I/PI assay (data not shown). Of the 1582 FDA-approved drugs tested, 23 drugs were found to have higher activity against the round body form of B. burgdorferi than doxycycline (Table 5). Among the 23 hits that were more active than doxycycline, 11 drugs, daptomycin, artemisinin, ciprofloxacin, sulfacetamide, sulfamethoxypyridazine, nifuroxime, fosfomycin, chlortetracycline, sulfathiazole, clofazimine and cefmenoxime, in order of decreasing activity, had better activity than the known round body active antibiotic metronidazole or tinidazole (Table 5). Antimalarial drug artemisinin showed high activity against the round body form of B. burgdorferi. Interestingly, ciprofloxacin (28% residual live cells) was the most active among quinolone drugs levofloxacin 41%, norfloxacin 41%, and moxifloxacin 49% (not shown). In addition, chlortetracycline, meclocycline and rolitetracycline were more active than doxycycline (42% residual live cells) against the round body forms (Table 5). On the other hand, some cell wall inhibitors such as vancomycin and macrolide antibiotic carbomycin, which had reasonable activity against stationary phase B. burgdorferi in the previous study (Feng, Wang, Shi, et al., 2014) showed relatively weak activity against the round body form of B. burgdorferi with 38% and 43% residual live cells, respectively (data not shown).

TABLE 5
Activity of top 23 active hits that had good activity against round body form
of B. burgdorferi^{a}

| | Residual viable | Ratios of green/red fluorescence | | | |
|---|---|---|---|---|---|
| Drugs (50 μM) | cells^{b} | Primary screening | Rescreening | Rescreening | p-value^{c} |
| Amoxicillin | 46% | 6.53 | 6.59 | 6.52 | 1.0000 |
| Doxycycline | 42% | 6.34 | 6.39 | 6.67 | 0.4915 |
| Penicillin G | 38% | 6.33 | 6.51 | 6.33 | 0.0767 |
| Cefuroxime | 34% | 6.29 | 6.28 | 6.31 | 0.0005 |
| Ceftriaxone | 36% | 6.37 | 6.29 | 6.39 | 0.0069 |
| Azithromycin | 47% | 6.79 | 6.52 | 6.42 | 0.8116 |
| Metronidazole | 33% | 6.23 | 6.30 | 6.31 | 0.0014 |
| Tinidazole | 33% | 6.24 | 6.21 | 6.36 | 0.0059 |
| Daptomycin^{d} | 19% | 5.90 | 6.09 | 5.93 | 0.0008 |
| Artemisinin | 24% | 5.96 | 6.14 | 6.17 | 0.0028 |
| Ciprofloxacin | 28% | 6.30 | 6.04 | 6.20 | 0.0108 |
| Sulfacetamide | 29% | 6.26 | 6.14 | 6.20 | 0.0011 |
| Sulfamethoxypyridazine | 30% | 6.20 | 6.07 | 6.34 | 0.0149 |
| Nifuroxime | 30% | 6.10 | 6.32 | 6.22 | 0.0079 |
| Fosfomycin | 31% | 6.39 | 6.12 | 6.16 | 0.0191 |
| Chlortetracycline | 31% | 6.20 | 6.36 | 6.18 | 0.0078 |
| Sulfathiazole | 31% | 6.38 | 6.18 | 6.17 | 0.0141 |
| Clofazimine | 32% | 6.29 | 6.22 | 6.24 | 0.0008 |
| Cefmenoxime | 32% | 6.18 | 6.33 | 6.28 | 0.0050 |
| Meclocycline | 33% | 6.52 | 6.23 | 6.09 | 0.1056 |
| Cefmetazole | 33% | 6.13 | 6.25 | 6.46 | 0.0530 |
| Loracarbef | 33% | 6.35 | 6.25 | 6.24 | 0.0036 |
| Sisomicin | 33% | 6.27 | 6.12 | 6.45 | 0.0562 |
| Sulfisoxazole | 33% | 6.53 | 6.15 | 6.18 | 0.1005 |
| Cefazolin | 34% | 6.23 | 6.34 | 6.29 | 0.0026 |
| Aztreonam | 34% | 6.16 | 6.38 | 6.33 | 0.0211 |
| Thymol | 34% | 6.15 | 6.36 | 6.36 | 0.0257 |
| Cefixime | 34% | 6.41 | 6.21 | 6.27 | 0.0169 |
| Sulfanilate | 34% | 6.49 | 6.03 | 6.40 | 0.1649 |
| Ceftazidime | 34% | 6.20 | 6.34 | 6.37 | 0.0126 |
| Rolitetracycline | 35% | 6.23 | 6.35 | 6.37 | 0.0085 |

^{a}Round body form of B. burgdorferi from 7-day-old culture was treated with FDA-approved drugs (50 μM) for 7 days. The line above tinidazole refers to antibiotics used to treat Lyme disease.
^{b}Residual viable B. burgdorferi was calculated according to the regression equation and ratios of Green/Red fluorescence obtained by SYBR Green I/PI assay.
^{c}p-values of standard t-test were calculated for the select antibiotic treated samples in comparison with the amoxicillin treated sample as a control.
^{d}Bold type indicates the 11 drug candidates that had better activity against the round body forms than metronidazole or tinidazole in order of decreasing activity.

MIC Values of Round Body Active Antibiotics:

In the previous study, it was found that the activity of antibiotics against non-growing persisters was not always correlated with their activity against growing B. burgdorferi (Feng, Wang, Shi, et al., 2014). Therefore, the MICs of artemisinin and ciprofloxacin that have excellent activity were tested against the round body form of B. burgdorferi using the SYBR Green I/PI assay and microscope counting. The MIC value of artemisinin was quite high at 50-100 μg/mL, indicating that artemisinin is much less active against growing B. burgdorferi, despite its high activity against the non-growing round body form of B. burgdorferi persisters. In contrast, ciprofloxacin was quite active against the growing B. burgdorferi with a low MIC (0.8-1.6 μg/mL), which is in agreement with a previous study (Kraiczy et al., 2001), indicating that it is quite active against both growing form and non-growing round body form of B. burgdorferi.

Effect of Drug Combinations on the Round Body Form and the Stationary Phase B. burgdorferi Persisters:

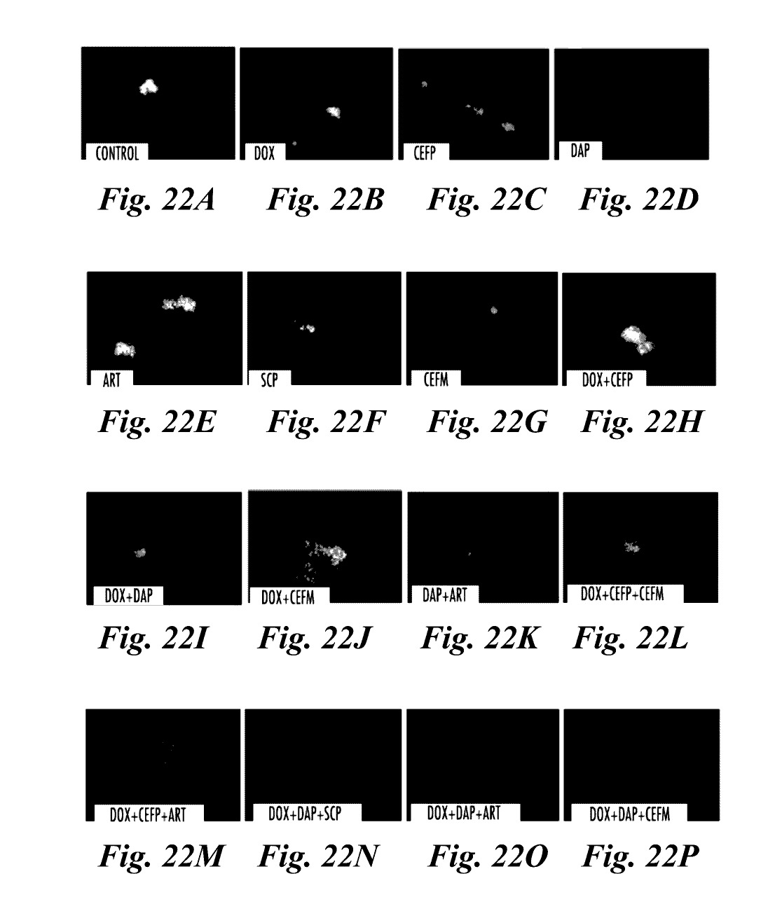

In the previous study (Feng, Wang, Shi, et al., 2014), it was found that stationary phase cultures are enriched with morphological variants such as round body form and biofilm-like aggregated micro-colony form. These morphological variant forms of B. burgdorferi have different antibiotic susceptibilities (Brorson et al., 2009; Sapi et al., 2011), and the recent study showed that drug combinations are more effective at killing the B. burgdorferi persisters than single drugs (Feng et al., submitted). To identify the best drug combinations with the active hits from the above screens against the round body form of B. burgdorferi, some active hits were evaluated including artemisinin, cefmetazole, and sulfachlorpyridazine in combination with promising FDA-approved drugs daptomycin or cefoperazone from the previous persister screen (Feng, Wang, Shi, et al., 2014) and current Lyme antibiotic doxycycline on the round body form and also on a stationary phase B. burgdorferi culture. The results showed that the drug combinations were much more effective than each of these drugs alone (Table 6, FIG. 22). Overall, the round body forms were more susceptible to the tested drugs or drug combinations than the 10 day old stationary phase culture which was enriched with more resistant microcolony forms (Table 6). It is worth noting that antimalarial drug artemisinin highlighted itself as having among the best activity against the stationary phase B. burgdorferi persisters when combined with other drugs. For example, artemisinin in combination with doxycycline and cefoperazone showed remarkable activity against the stationary phase B. burgdorferi persisters (Table 6, FIG. 22o). Cefmenoxime and cefmetazole were the most effective of the few cephalosporin drugs tested against the round body form of B. burgdorferi. In addition, it was noted that the sulfa drug sulfachlorpyridazine when combined with daptomycin and doxycycline showed remarkable activity against B. burgdorferi persisters (FIG. 22n). Moreover, sulfachlorpyridazole combined with doxycycline and daptomycin showed the best activity against the round body form of B. burgdorferi persisters (Table 6).

TABLE 6
Effect of drug combinations on the round body form and stationary phase
culture (10 day old) of B. burgdorferi

| | Live cell % | CefM | Scp | Art | Nft |
|---|---|---|---|---|---|
| Control | 50% (87%) | 34% (53%) | 38% (68%) | 28% (73%) | 33% (82%) |
| Dox | 49% (72%) | 31% (43%) | 29% (62%) | 26% (64%) | 30% (74%) |
| CefP | 30% (64%) | 31% (41%) | 30% (55%) | 25% (42%) | 25% (56%) |
| Dox + CefP | 29% (59%) | 28% (41%) | 25% (69%) | 23% (24%) | 23% (52%) |
| DAP | 17% (48%) | 16% (20%) | 15% (27%) | 24% (29%) | 16% (33%) |
| DAP + Dox | 16% (34%) | 16% (16%) | 8% (21%) | 15% (19%) | 17% (20%) |

Round body form and ten day old stationary phase (in brackets) B. burgdorferi culture was treated with 10 μg/mL drugs or their combinations for 7 days. Residual viable B. burgdorferi was calculated according to the regression equation and ratio of Green/Red fluorescence obtained by SYBR Green I/PI assay as described (Feng, Wang, Shi, et al., 2014). Direct microscopy counting was employed to rectify the results of the SYBR Green I/PI assay. Residual viable percentages less than 30% are shown in bold text and the best drug combinations without daptomycin are underlined. Abbreviation: Dox, doxcycline; CefP, cefoperazone; DAP, daptomycin; CefM, cefmetazole; Scp, sulfachlorpyridazine; Art, artemisinin; Nft, nitrofurantoin.

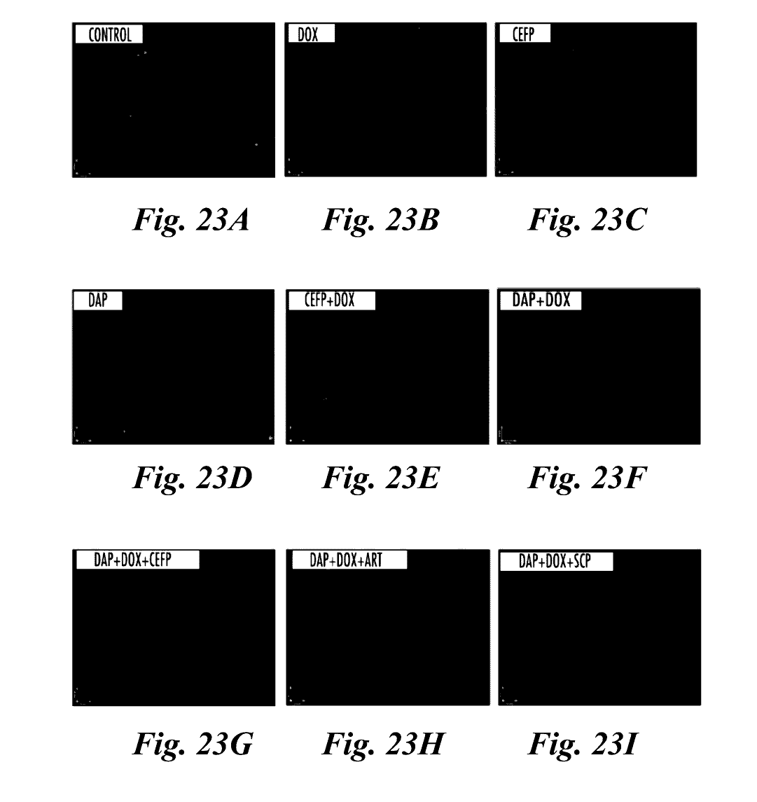

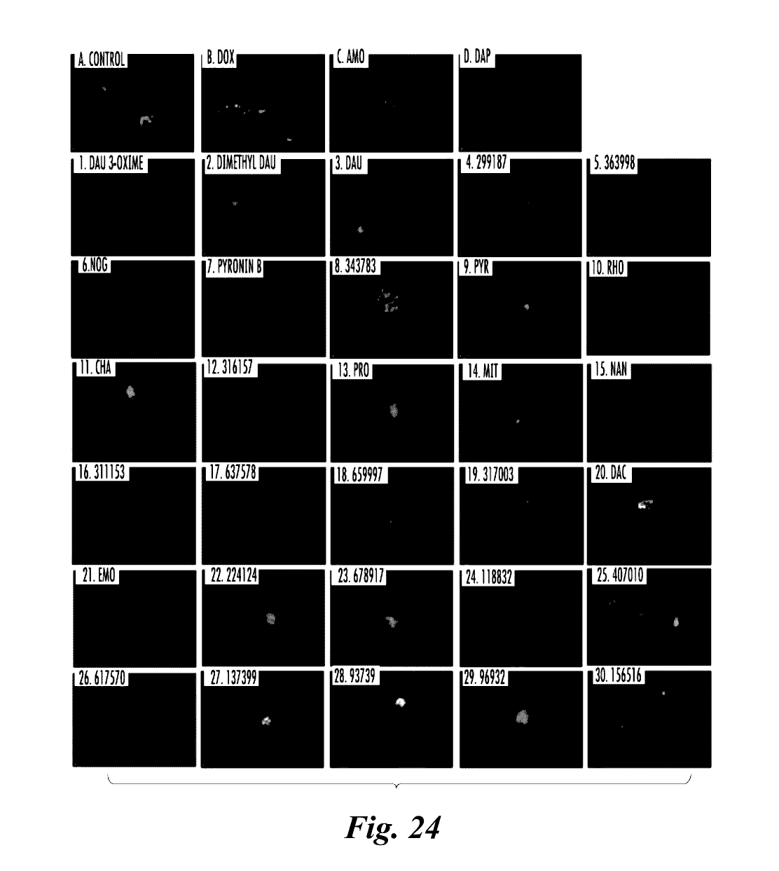

To confirm the drug combination results, subculture studies were performed in fresh BSK-H medium as described in the previous study (Feng, et al., in press) and it was found that drug-free round body controls and samples treated with single drugs grew in 10 day subculture (Table 7). Samples treated with two drug combinations grew more slowly (Table 7). However, after the 10 day subculture, the three drug combinations, e.g., doxycycline+daptomycin+ either cefoperazone or artemisinin or sulfachlorpyridazine did not show any sign of growth as no visible spirochetes were observed, whereas other drug combinations all had visible live spirochetes under the microscope (data not shown). After the 20-day subculture, there were about 8×10⁶ spirochetes in the control sample and about 5×10⁶ spirochetes in doxycycline-treated samples (Table 7). Daptomycin alone, or two drug combinations doxycycline+cefoperazone and doxycycline+daptomycin could not sterilize the round body form of B. burgdorferi persisters, as they all had visible spirochetes growing after the 20-day subculture (FIG. 23). However, doxycycline+daptomycin+artemisinin or sulfachlorpyridazine significantly reduced the number of spirochetes with very few spirochetes being visible after the 20-day subculture (FIG. 23). The daptomycin in combination with doxycycline and cefoperazone still showed the best activity which killed all round body form of B. burgdorferi persisters with no viable spirochetes observed after the 20-day subculture (FIG. 23).

TABLE 7
Subculture tests to assess the viability of drug-treated round
body form of B. burgdorferi^{a}

| | | | Spirochetes |
|---|---|---|---|
| | | Spirochetes number | number |
| | Residual | After 10 day | After 20 day |
| Drugs^{b} | viable cells^{c} | subculture^{d} | subculture^{d} |
| Control | 49% | 2 × 10⁶ | 8 × 10⁶ |
| Dox | 44% | 1 × 10⁶ | 5 × 10⁶ |
| Dox + Dap + CefP | 13% | <1 × 10⁵ | <1 × 10^{5e} |
| Dox + Dap + Art | 15% | <1 × 10⁵ | 3 × 10⁵ |
| Dox + Dap + Scp | 17% | <1 × 10⁵ | 6 × 10⁵ |
| Dox + CefP | 31% | 7.5 × 10⁵ | 4 × 10⁶ |
| Dap | 18% | 5 × 10⁵ | 5 × 10⁶ |
| CefP | 33% | 9 × 10⁵ | 4 × 10⁶ |

^{a}Amoxicillin induced rounded body form B. burgdorferi culture (500 μL) was treated with 10 μg/mL drugs alone or drug combinations for 7 days. Then, 50 μL of washed bacterial cells was subcultured in 1 mL fresh BSK-H medium for 10 days and 20 days, respectively and examined by microscopy.
^{b}Abbreviations: Dox, doxycycline; CefP, cefoperazone; Dap, daptomycin; Art, artemisinin; Scp, sulfachlorpyridazine
^{c}Green/Red fluorescence ratios were obtained by microplate reader after SYBR Green I/PI staining Each value is the mean of three replicates.
^{d}The number of spirochetes was evaluated by microscope count.
^{e}Below detection limit as shown by lack of any visible spirochetes by microscopy.

### Discussion

Previous in vitro and in vivo studies showed the round body form of B. burgdorferi as a persister form could survive in adverse conditions including antibiotic exposure in vitro and are found in chronic Lyme neuroborreliosis in vivo (Brorson and Brorson, 1998; Miklossy et al., 2008). As shown in previous studies (Brorson and Brorson, 1997; Murgia and Cinco, 2004; Brorson and Brorson, 1998) and also in this study, the round body form of B. burgdorferi could still reproduce and revert to spirochetes under suitable conditions upon removal of the stress during subculture. The B. burgdorferi round body form shows lower metabolic activity and is tolerant to antibiotics (Brorson et al., 2009; Kersten et al., 1995; Barthold et al., 2010). Although metronidazole, tinidazole and tigecycline were reported to have certain activity against the round body form, they were not able to completely eradicate these persister forms (Sapi et al., 2011). Thus, no good antibiotics against the round body form of B. burgdorferi are available (Brorson et al., 2009; Barthold et al., 2010). These studies demonstrate that it would be very difficult to kill the round body form of B. burgdorferi using current antibiotics.

In this study, this problem was addressed by first establishing an amoxicillin-induced round body model for B. burgdorferi persisters and then screening an FDA-approved drug library for activities against the round body form of B. burgdorferi. Eleven drug candidates were identified that have better activity against the round body form of B. burgdorferi (Table 5) than metronidazole or tinidazole, a control drug that is known to be active against the round body form (Sapi et al., 2011; Brorson and Brorson, 1999).

In a previous study, several drugs were identified that show excellent activity for stationary phase B. burgdorferi persisters from an FDA-approved drug library (Feng, Wang, Shi, et al., 2014). Some hits against the stationary phase B. burgdorferi persisters are also identified in this screen against round body form such as daptomycin, clofazimine and sulfa drugs, which validates the previous finding that these agents are active against the persister forms. Importantly, some active antibiotics against the round body form B. burgdorferi were found that did not show good activity in the previous drug screen against stationary phase B. burgdorferi persisters (Feng, Wang, Shi, et al., 2014). These include artemisinin, ciprofloxacin, nifuroxime, fosfomycin, tinidazole, loracarbef, and thymol that appear to be specifically active against the round body form. These candidate drugs are FDA approved and used for treatment of infections other than Lyme disease.

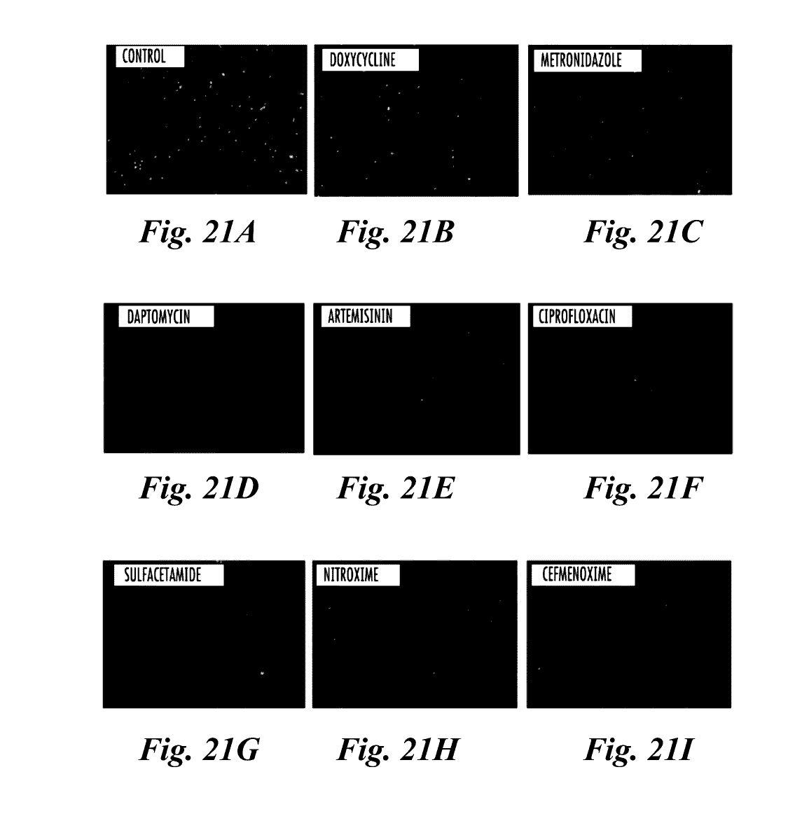

As in the previous study (Feng, Wang, Shi, et al., 2014), daptomycin still remains the most active drug against the round body form of B. burgdorferi persisters. Daptomycin killed most planktonic round body form of B. burgdorferi (FIG. 21d). It is possible that daptomycin preferentially acts on the membrane of the round body form of B. burgdorferi that is different from the membrane of actively growing spirochetal form and thus making it particularly active for the persister forms. Daptomycin is known to disrupt the membrane structure and cause rapid depolarization thus depleting membrane energy that may be required for viability of the persisters.

An interesting finding of the study is the observation that the antimalarial drug artemisinin showed excellent activity against the round body form of B. burgdorferi persisters (FIG. 21e). It is worth noting that artemisinin while having a high MIC (50-100 μg/mL) showed excellent activity against the round body form of B. burgdorferi compared with most commonly used antibiotics. Artemisinin is a commonly used antimalarial drug isolated from the plant Artemisia annua, a Chinese herbal medicine. The mechanism of action of artemisinin is not well understood. The antimalarial activity of artemisinin might involve endoperoxide activation by free ferrous iron from haemoglobin digestion by malaria parasites (Wells et al., 2009). However, the content of ferrous iron or haemoglobin is very low in the B. burgdorferi culture, so the activation of endoperoxide might not be the main mechanism of artemisinin activity against B. burgdorferi round body forms. The study in yeast is noted in which artemisinin impairs the membrane structure and causes depolarization of the mitochondrial membrane (Wang, Huang, et al., 2010; Li et al., 2005). In this respect, it is possible that artemisinin may have a similar mechanism of action of disrupting the bacterial membrane as the basis for its high activity against the round body form of B. burgdorferi. It is noteworthy that artemisinin has been used for treating Lyme co-infections and found to be effective clinically. The reason that artemisinin is effective was interpreted to be due to its action against Babesia co-infection, but it is quite likely that the clinical efficacy of artemisinin may at least partly be due to its activity against the round body form of B. burgdorferi persisters as shown in this study.

It was previously found that daptomycin combined with doxycycline and cefoperazone could best eliminate the most resistant microcolony form of persisters (Feng et al., submitted). In this study, it was found that artemisinin was the best substitute for daptomycin in the drug combination with doxycycline and cefoperazone and showed excellent activity against round body form of persisters (Table 6, FIG. 22m). Also, the lipophilic antibiotic clofazimine, which has complex antimicrobial activity including membrane disruption and depolarization (Van Rensburg et al., 1992; Cholo et al., 2012), showed good activity against both round body form and stationary phase persisters. Based on these findings on daptomycin, artemisinin and clofazimine, and without wishing to be bound to any one particular theory, it is proposed that membrane disruption may be a good approach to killing B. burgdorferi persisters.

Besides the top-ranked hits of screened drugs, many sulfonamide antibiotics, such as sulfacetamide, sulfamethoxypyridazine and sulfaquinoxaline, were found to be highly active against the round body form (Table 5). The sulfonamide antibiotics have also been identified in the previous drug screen against stationary phase persisters and showed low MICs (<0.2 μg/mL) (Feng, Wang, Shi, et al., 2014). The sulfonamides inhibit utilization of PABA required for the synthesis of folic acid, which results in the blockade of several enzymes needed for synthesis of DNA and methionine, glycine, and formylmethionyl-transfer-RNA. It is worth noting that sulfachloropyridazine as the analogue of sulfamethoxypyridazine also showed good activity against stationary phase B. burgdorferi (residual viable cells is about 38%) and, when combined with daptomycin and doxycycline, showed remarkable activity against stationary phase B. burgdorferi (residual viable cells is about 8%) (Table 6). It is believed that further studies on metabolic changes of the round body form of B. burgdorferi could help understand the mechanism by which sulfonamide acts against B. burgdorferi persisters.

In addition to the previously found drugs (Feng, Wang, Zhang, et al., 2014), some novel drugs were discovered to be preferentially active against the round body form of B. burgdorferi in this study. Ciprofloxacin as a fluoroquinolone has shown activity against B. burgdorferi in vitro and could kill the inoculum with 16 μg/mL MBC (41.5 μM) after 72 h (Kraiczy et al., 2001). It has been presently disclosed that ciprofloxacin was the most active fluoroquinolone against the round body form of B. burgdorferi among other quinolones, but ciprofloxacin was not identified to have activity against B. burgdorferi stationary phase persisters in the previous screen (Feng, Wang, Shi, et al., 2014) as it was not in the old version of the FDA drug library. However, ciprofloxacin (50 μM) alone could not completely kill the round body form after 7 days. This result indicates that the round body form is more resistant or tolerant to antibiotics than multiplying B. burgdorferi. On the other hand, some drugs, such as nifuroxime and thymol, did not show activity in the previous drug screen on stationary phase B. burgdorferi, but showed good activity against the round body form in this study. This specific activity against the round body form could be related to the physiological difference of different morphological forms and/or the synergistic activity of these drugs with amoxicillin used to induce round forms used for drug screens. It is of interest to note that chlortetracycline was more active than doxycycline against the round body forms and that nitrofuran derivative nifuroxime was more active than metronidazole or tinidazole (Table 5). These findings could indicate the side chain involved in both cases may have conferred additional activity against the round body persisters. The drug combination test on the stationary phase B. burgdorferi using the nifuroxime analogue nitrofurantoin (residual live cell is 39%) showed that nitrofurantoin combined with cefoperazone was more effective than each drug alone (Table 6). Likewise, natural antimicrobial thymol combined with amoxicillin showed good activity (residual percentage is 34%) in the round body drug screen, but thymol alone did not work on the stationary phase B. burgdorferi (residual live cell percentage is 82%) in the previous drug screen. Palaniappan et al. reported that thymol could reduce the resistance in E. coli and S. aureus to ampicillin and penicillin (2010). This synergistic activity between thymol and beta-lactams may explain its activity against the B. burgdorferi round body form. These results suggest that the drug combination could be an effective approach to fighting against B. burgdorferi persisters.

However, some drugs that had activity against stationary phase B. burgdorferi, such as beta-lactams, vancomycin, streptomycin, and amphotericin B (Feng, Wang, Shi, et al., 2014) did not show good activity against the round body form (Table 5). However, it was noted that two cephalosporins, cefmenoxime and cefmetazole, showed good activity against the round body form of B. burgdorferi. In the previous drug screen on stationary phase B. burgdorferi, cefoperazone, which was the best cephalosporin for killing stationary phase B. burgdorferi, also had certain activity against the round body form (not shown). Future studies are needed to further explore the mechanism of action of these cephalosporins that have activity against B. burgdorferi persisters which may involve targets outside the cell wall synthesis. Vancomycin is a glycopeptide antibiotic acting on the cell wall rather than acting on the cell membrane like daptomycin. Good activity of vancomycin was not found against amoxicillin treated round bodies, though it showed relatively good activity against stationary phase B. burgdorferi in the previous drug screen (Feng, Wang, Shi, et al., 2014). This might be due to cell wall deficiency of the round body form induced by amoxicillin.

In summary, this study represents the first high-throughput drug screens against the round body form of B. burgdorferi persisters and identified a number of FDA-approved antibiotics that show excellent activity against such forms. Despite some overlap in drugs active against both stationary phase persisters and round body form of persisters, some interesting drug candidates were identified that are preferentially active against the round body form of persisters, including artemisinin, ciprofloxacin, nifuroxime, fosfomycin, chlortetracycline, and some sulfa drugs which were found to be active against the round body form for the first time. These round body effective drugs in appropriate combinations can be used to eliminate the persistence phenomenon and improve the treatment of persistent forms of Lyme disease, including antibiotic refractory Lyme arthritis and PTLDS.

### Example 4

### Identification of New Compounds with High Activity Against Borrelia burgdorferi Persisters from the NCI Compound Collection

### Materials and Methods

Bacterial Strain, Media and Culture:

Borrelia burgdorferi strain B31 (ATCC 35210) was obtained from American Type Tissue Collection. B. burgdorferi was cultured in BSK-H medium (HiMedia Laboratories Pvt. Ltd.), with 6% rabbit serum (Sigma-Aldrich). All culture media were filter-sterilized by 0.2 μm filter. Cultures were incubated in sterile 50 mL closed conical tubes (BD Biosciences, California, USA) at 33° C. without antibiotics. Based on a previous study that demonstrated the antibiotic tolerance of the stationary phase cultures, 7 day old stationary phase B. burgdorferi cultures enriched in persisters were chosen for drug screens in 96-well microtiter plates as described (Feng, Wang, Shi, et al., 2014).

Microscopy Techniques:

Specimens were examined on a Zeiss Axiolmager M2 microscope equipped with differential interference contrast (DIC) and epifluorescent illumination, and recorded with a Hamamatsu ORCA-R² C10600 camera. Cell proliferation assay was performed by direct counting using a bacterial counting chamber (Hausser Scientific Partnership, PA, USA) and DIC microscopy. SYBR Green I/PI assay was performed to assess the viability of B. burgdorferi as described (Feng, Wang, Zhang, et al., 2014). The ratio of live (green) and dead (red) B. burgdorferi was calculated by counting these cells using a bacterial counting chamber and epi-fluorescence microscopy as previously described (Feng, Wang, Zhang, et al., 2014).

Antibiotics and the NCI chemical compound library: Antibiotics including doxycycline, amoxicillin, and daptomycin were purchased from Sigma-Aldrich and dissolved in appropriate solvents (Clinical and Laboratory Standards Institute, 2007) to form stock solutions. All the antibiotic stocks were filter-sterilized by 0.2 μm filter. Then the stocks were diluted into 500 μM pre-diluted stocks and stored at −20° C.

The NCI compound library collection, consisting of diversity set V (Moody et al., 1978), mechanistic diversity set II (DTP-Mechanistic Set Information, 2015) and the natural products set III (DTP-Natural Products Set Information, 2015), was kindly supplied by National Cancer Institute Developmental Therapeutic Program's Open Compound Repository. These NCI compound libraries were prepared in 1 mM stock solutions with DMSO in 96-well plates leaving the first and the last columns in each plate for controls, which included DMSO blank controls, doxycycline control, and amoxicillin control. The pre-diluted drug plates were sealed and stored at −20° C.

Screening NCI Compound Libraries Against B. burgdorferi Stationary Phase Persisters:

To qualitatively determine the effect of compounds on B. burgdorferi persisters, each compound (5 μL) from the pre-diluted stocks was added to a 7 day old B. burgdorferi stationary phase culture in 96-well microtiter plates. The final volume per well was adjusted to 100 μL to achieve a final drug library concentration of 50 μM in the drug screen. The plates were sealed and placed in a 33° C. incubator for 7 days when the viability of the bacteria was assessed by SYBR Green I/PI assay as described in a previous study (Feng, Wang, Zhang, et al., 2014). With the excitation wavelength at 485 nm, the fluorescence intensities at 535 nm (green emission) and 615 nm (red emission) were measured for each well of the screening plate using SpectraMax M2 Microplate Reader (Molecular Devices Inc., USA). Some effective candidates were further confirmed by epifluorescence microscopy as described (Feng, Wang, Zhang, et al., 2014).

MIC Determination:

The standard microdilution method was used to determine the minimum inhibitory concentration (MIC) that would inhibit visible growth of B. burgdorferi after a 72 hours incubation period (Sapi et al., 2011; Dever et al., 1992; Boerner et al., 1995). B. burgdorferi cells (1×10⁵) were inoculated into each well of a 96-well microplate containing 90 μL fresh BSK-H medium per well. Each diluted compound (10 μL) was added to the culture. All experiments were run in triplicate. The 96-well plate was sealed and placed in an incubator at 33° C. for 5 days. Cell proliferation was assessed using the SYBR Green I/PI assay and a bacterial counting chamber after the incubation as described (Feng, Wang, Shi, et al., 2014).

### Introduction

To identify drugs that can more effectively kill B. burgdorferi persisters, a new viability assay using SYBR Green I/propidium iodide (PI) dyes was recently developed (Feng, Wang, Zhang, et al., 2014), which allowed screening of a FDA-approved drug library against stationary phase B. burgdorferi persisters (Feng, Wang, Zhang, et al., 2014). Using this high-throughput assay, a number of interesting drug candidates were identified, such as daptomycin, clofazimine, cefoperazone, carbomycin, which have excellent activity against in vitro B. burgdorferi persisters (Feng, Wang, Shi, et al., 2014). In the previous study, daptomycin was found to have the highest activity against B. burgdorferi persisters among all the candidate drugs. Although daptomycin could almost eradicate B. burgdorferi persisters at 50 μM, this drug concentration is quite high for clinical use, and in addition, daptomycin generally has to be used intravenously, which is not convenient to administer.

To identify new and more effective drugs than daptomycin in killing B. burgdorferi persisters, in this example, new drug screens were performed on stationary phase B. burgdorferi persisters using the chemical repository collection of the National Cancer Institute (NCI compound library collection). This NCI compound library collection has three compound libraries: the diversity set IV compound library (1593 compounds), the mechanistic set II library (816 compounds), and the natural product set III library (117 compounds), for a total of 2526 compounds. These compounds are chosen based on structural diversity from more than 250,000 natural products and synthetic compounds (Open Repository Collection of Synthetics and Pure Natural Products, 2014). By screening this NCI compound library collection, new anti-persister compounds were identified that were not found in the previous screens (Feng, Wang, Shi, et al., 2014). These new persister active hits can be used for a treatment for Lyme disease.

### Results

Screening NCI Compound Library to Identify Effective Drugs Active Against Dormant B. burgdorferi Persisters:

The SYBR Green I/PI assay was used as a high-throughput screening method for rapid viability assessment for B. burgdorferi after exposure to the compound libraries (Feng, Wang, Shi, et al., 2014). Based on a previous study, some red colored compounds caused interference to the SYBR Green I/PI assay, which could make the background red and cause false positive results. Thus, in the presently disclosed subject matter, microscopic counting rescreens were done to examine the hit compounds in SYBR Green I/PI assay.

To identify effective chemical compounds that have activity against B. burgdorferi persisters, stationary phase B. burgdorferi was used as a persistence model to screen the NCI compound libraries. Meanwhile, the currently used Lyme disease antibiotics doxycycline and amoxicillin were included as control drugs. Consistent with previous results (Feng, Wang, Shi, et al., 2014), the currently used Lyme antibiotics had poor activity against the stationary phase B. burgdorferi persisters, and the bacteria treated with the two antibiotics still had 75% and 76% viable cells remaining, respectively, compared with 93% viable cells in drug-free control (Table 8).

Of the 2526 compounds in the NCI compound library collection tested, 237 were found to have higher activity against B. burgdorferi persisters than doxycycline and amoxicillin in the primary screen. The 237 candidates were rescreened by microscope counting with the SYBR Green I/PI viability assay. After the rescreen by microscopy, the top 30 active hits that had less than 50% residual viable cells after treatment were confirmed (Table 8, FIG. 24). Among the 30 active hits, 22 compounds were found in Mechanistic Set II, 9 compounds in Diversity Set IV, and 3 compounds in Natural Product Set III. Nanaomycin and dactinomycin showed up in both Mechanistic Set II and Natural Product Set III and NSC311153 and NSC637578 in both Mechanistic Set II and Diversity Set IV. It is interesting to note that all of them are aromatic compounds. Several clinically used drugs were identified that had excellent activity against stationary phase B. burgdorferi persisters. Anti-persister activities of some drugs were significantly higher than that of frontline antibiotics doxycycline or amoxicillin and even more active than daptomycin, the best antibiotic against B. burgdorferi persisters in a previous study (Table 8, FIG. 24). Six anthraquinone antibiotics and compounds, daunomycin 3-oxime, dimethyldaunomycin, daunomycin, NSC299187, NSC363998 and nogalamycin, showed the highest activities (residual viable cells from 6% to 15%) against stationary phase B. burgdorferi persisters. These six compounds showed higher activity than daptomycin (18% residual viable cells). In addition, another five anthraquinone compounds, pyrromycin, rhodomycin A, NCS316157, emodin, and NSC156516, also showed good activity against stationary phase B. burgdorferi persisters (residual viable cells 21% to 50%). Following the six anthraquinones, pyronin B, a xanthene compound highlighted itself as having a good activity (residual viable cells 19%) against stationary phase B. burgdorferi persisters. Seven nitrogen-containing aromatic compounds, NSC343783 (residual viable cells 20%), Prodigiosin (24% residual viable cells), NSC637578, NSC 678917, NSC118832, NSC617570 and NSC96932, were found to be among the 30 most active compounds. Moreover, chaetochromin, a bis-naphtho-γ-pyrone compound, showed good activity with 22% residual viable cells. Mitomycin, an aziridine-containing benzoquinone antitumour drug, showed reasonably good activity with 25% residual viable cells. Three 1,4-naphthoquinones, nanaomycin (residual viable cells 26%), NCS659997 and NCS224124, had relatively good activity against stationary phase B. burgdorferi persisters. A polypeptide antibiotic dactinomycin also had relatively high activity against B. burgdorferi persisters (residual viable cells 30%). Besides 11 clinically used drugs (daunomycin 3-oxime, dimethyldaunomycin, daunomycin, nogalamycin, pyrromycin, chaetochromin, prodigiosin, mitomycin, nanaomycin and dactinomycin), 19 non-medicinal compounds also were found that showed good activity against stationary phase B. burgdorferi persisters to varying levels (Table 8, FIG. 24).

TABLE 8
Activity of top 30 active hits that had good activity (better than current
clinical drugs) against stationary phase B. burgdorferi persisters^{a}

| | | | Residual | Residual |
|---|---|---|---|---|
| | | | viable | viable |
| NSC^{b} | Compounds (50 μM) | Chemical structure | cells^{c} | cells^{d} |
| | Control | — | 93% | 95% |
| | Amoxicillin | — | 71% | 77% |
| | Doxycycline | — | 68% | 77% |
| | Daptomycn | — | 23% | 18% |
| 143491 | Daunomycin 3-oxime hydrochloride | 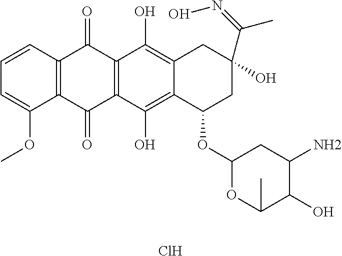 | 0% | 6% |
| 258812 | Dimethyldaunomycin hydrochloride | 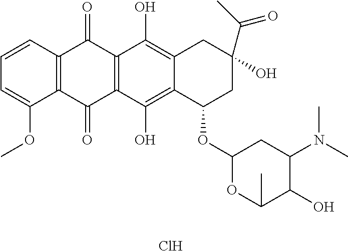 | 0% | 10% |
| 82151 | Daunorubicin hydrochloride | 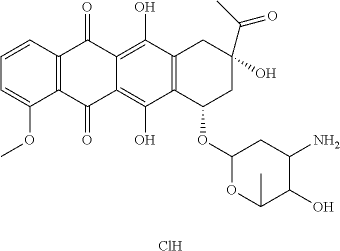 | 0% | 10% |
| 299187 | 9,10-Anthracenedione, 1- hydroxy-4-[[2-[(2- hydroxyethyl)amino]ethyl] amino]- | 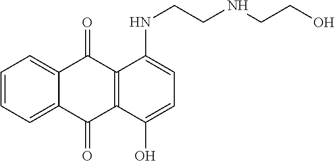 [SMILES: O=C1c2ccccc2C(=O)c2c(NCCNCCO)ccc(O)c21] | 5% | 13% |
| 363998 | Anthracene-9,10-dione, 1,5-bis[3-[[(2- hydroxyethyl)amino] propyl]amino]-9,10- dihydro-, dihydrochloride | 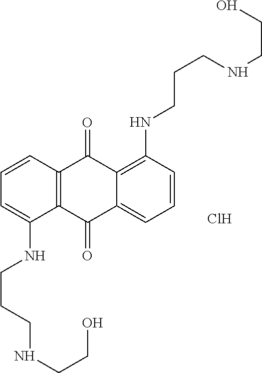 [SMILES: O=C1c2cccc(NCCCNCCO)c2C(=O)c2cccc(NCCCNCCO)c21 ; Cl] | 0% | 13% |
| 70845 | Nogalamycin | 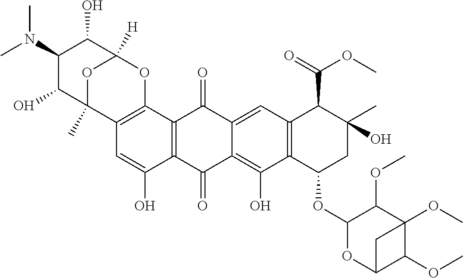 | 0% | 15% |
| 44690 | Pyronin B | 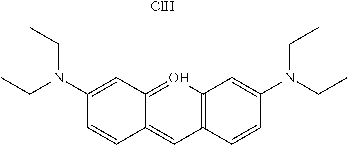 | 0% | 19% |
| 343783 | N-Allyl-2- (methylthio)[1,3]thiazolo [5,4-d]pyrimidin-7-amine | 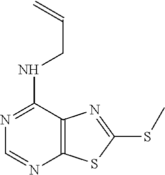 | 67% | 20% |
| 267229 | Pyrromycin | 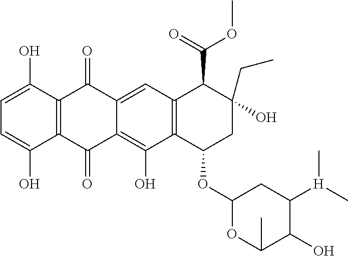 | 0% | 21% |
| 136044 | Rhodomycin A | 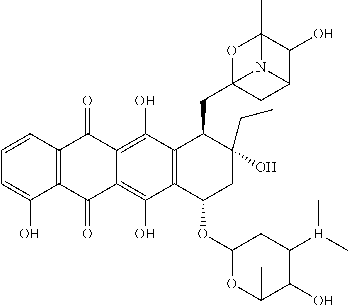 | 0% | 22% |
| 345647 | Chaetochromin | 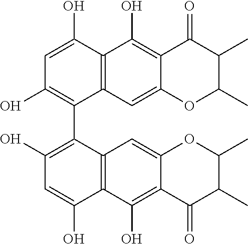 [SMILES: CC1Oc2cc3c(-c4c(O)cc(O)c5c(O)c6c(cc45)OC(C)C(C)C6=O)c(O)cc(O)c3c(O)c2C(=O)C1C] | 8% | 22% |
| 316157 | 9,10-Anthracenedione, 1,4-dihydroxy-2-[[2-[(2- hydroxyethyl)amino]ethyl] amino]- | 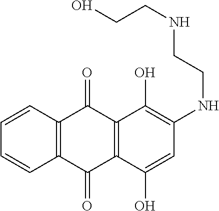 [SMILES: O=C1c2ccccc2C(=O)c2c(O)c(NCCNCCO)cc(O)c21] | 0% | 23% |
| 47147 | Prodigiosin | 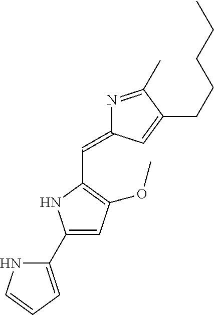 [SMILES: CCCCCC1=C/C(=C\c2[nH]c(-c3ccc[nH]3)cc2OC)N=C1C] | 0% | 24% |
| 26980 | Mitomycin | 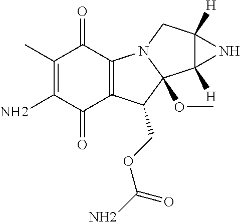 [SMILES: CO[C@@]12[C@H](COC(N)=O)C3=C(C(=O)C(C)=C(N)C3=O)N1C[C@@H]1N[C@@H]12] | 20% | 25% |
| 267461 | Nanaomycin | 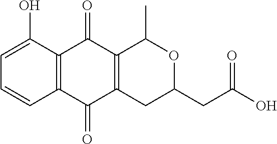 [SMILES: CC1OC(CC(=O)O)CC2=C1C(=O)c1c(O)cccc1C2=O] | 34% | 26% |
| 311153 | 9-Hydroxy-2-(2- piperidinylethyl)ellipticini- ium acetate | 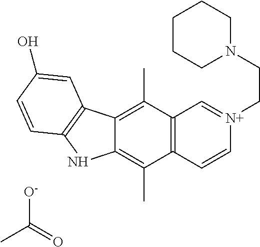 | 45% | 26% |
| 637578 | N-[3-(2- Pyridyl)isoquinolin-1-yl]- 2-pyridinecarboxamidine | 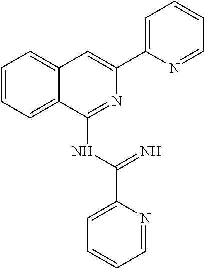 [SMILES: N=C(Nc1nc(-c2ccccn2)cc2ccccc12)c1ccccn1] | 46% | 26% |
| 659997 | Naphthalene-1,4-dione, 2- chloro-5,8-dihydroxy-3- (2-methoxyethoxy)- | 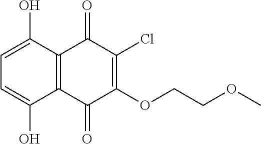 [SMILES: COCCOC1=C(Cl)C(=O)c2c(O)ccc(O)c2C1=O] | 1% | 28% |
| 317003 | 9H-Thioxanthen-9-one, 1- [[2- (dimethylamino)ethyl]ami- no]-7-hydroxy-4-methyl-, monohydriodide | 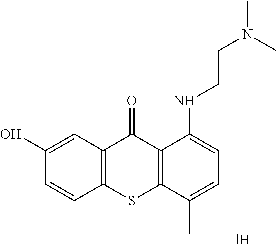 [SMILES: Cc1ccc(NCCN(C)C)c2c(=O)c3cc(O)ccc3sc12] | 29% | 30% |
| 3053 | Dactinomycin | 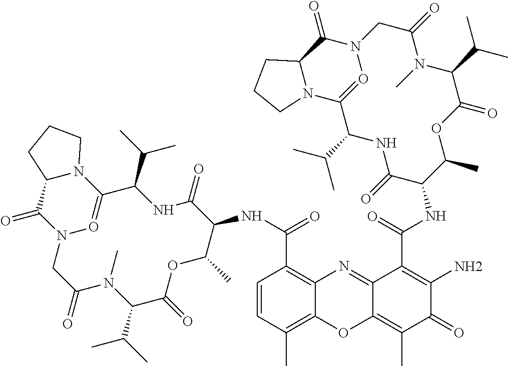 | 37% | 30% |
| 408120 | Emodin | 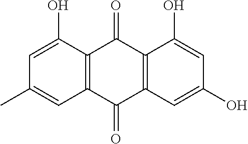 [SMILES: Cc1cc(O)c2c(c1)C(=O)c1cc(O)cc(O)c1C2=O] | 18% | 31% |
| 224124 | (5,8-dihydroxy-1,4-dioxo- 1,4-dihydronaphthalene- 2,3-diyl)dimethanediyl dicarbamate | 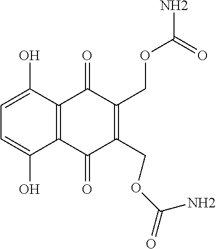 [SMILES: NC(=O)OCC1=C(COC(N)=O)C(=O)c2c(O)ccc(O)c2C1=O] | 32% | 31% |
| 678917 | 1-Phenazinecarboxamide, N-[2- (dimethylamino)ethyl]- 6,7-dimethoxy-, monohydrochloride | 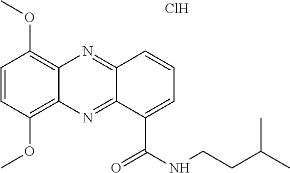 [SMILES: COc1ccc(OC)c2nc3c(C(=O)NCCC(C)C)cccc3nc12 ; Cl] | 44% | 35% |
| 118832 | (5-phenyl-1,3-thiazol-2- yl)methanol | 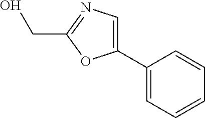 [SMILES: OCc1ncc(-c2ccccc2)o1] | 30% | 38% |
| 407010 | 2-(3,4-dihydroxyphenyl)- 3,7-dihydroxy-4H-1- benzopyran-4-one | 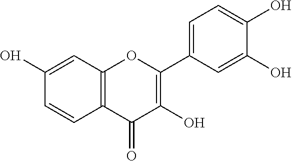 [SMILES: O=c1c(O)c(-c2ccc(O)c(O)c2)oc2cc(O)ccc12] | 39% | 38% |
| 617570 | Benzoic acid, 2-hydroxy-, (2,6- pyridinediyldiethylidyne) dihydrazide, nickel complex | 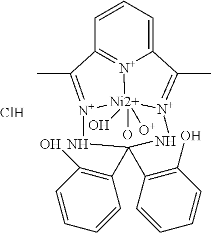 | 44% | 38% |
| 137399 | 1-(1,2-Dihydro-5- acenaphthylenyl)-N- hydroxy-1- phenylmethanimine | 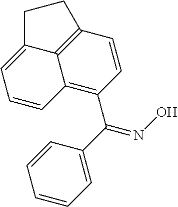 [SMILES: O/N=C(/c1ccccc1)c1ccc2c3c(cccc13)CC2] | 51% | 41% |
| 93739 | 2-Methyl-4,4′-[(4-imino- 2,5-cyclohexadien-1- ylidene)methylene]di- aniline hydrochloride | 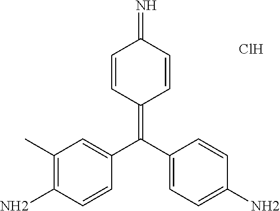 [SMILES: Cc1cc(C(=C2C=CC(=N)C=C2)c2ccc(N)cc2)ccc1N ; Cl] | 0% | 43% |
| 96932 | 3,3′-Diethyl-9- methylthiacarbocyanine iodide | 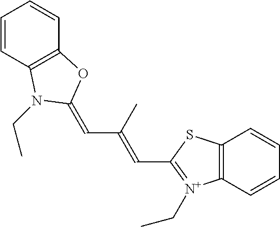 [SMILES: CCN1/C(=C/C(C)=C/c2sc3ccccc3[n+]2CC)Oc2ccccc21] | 0% | 46% |
| 156516 | 1,8- Di(phenylthio)anthraquin one | 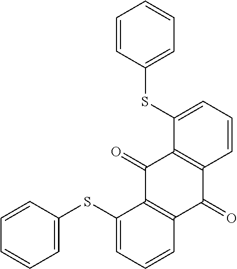 [SMILES: O=C1c2cccc(Sc3ccccc3)c2C(=O)c2c(Sc3ccccc3)cccc21] | 46% | 50% |

^{a}Seven day old stationary phase B. burgdorferi culture was treated with drugs or compounds (50 μM) for 7 days when the viability of the bacteria was determined as described (Feng, Wang. Shi, et al., 2014).
^{b}The NSC number is a numeric identifier for substances submitted to the National Cancer Institute (NCI).
^{c}Residual viable B. burgdorferi was calculated according to the regression equation and ratio of Green/Red fluorescence obtained by SYBR Green I/PI assay as described (Feng, Wang, Shi, et al., 2014).
^{d}Residual viable B. burgdorferi was assayed by epifluorescence microscope counting as described (Feng, Wang, Shi, et al., 2014).

Relationship Between MIC Values and Anti-Persister Activity:

Some compounds that have good activity against the non-growing stationary phase B. burgdorferi persisters were found (Table 8), but it is necessary to determine the MICs of these compounds against growing B. burgdorferi (Table 9). The standard microdilution method was used to determine the MIC as described in a previous study (Feng, Wang, Shi, et al., 2014). It was found that three anthracycline antibiotics, daunomycin 3-oxime, daunorubicin and pyrromycin, in addition to having good activity against stationary phase B. burgdorferi persisters, also were highly active against log phase growing B. burgdorferi with low MICs (≦0.36, ≦0.36, 0.36-0.72 g/mL, respectively). Another anthraquinone compound, NSC299187, showed relatively high MIC (3.26-6.52 μg/mL) although it had excellent anti-persister activity (residual viable cells 13%). It was also noted that prodigiosin (nitrogen-containing aromatic rings compound), mitomycin (aziridine-containing benzoquinone), nanaomycin (1,4-naphthoquinone) and dactinomycin (polypeptide antibiotic) had very good activity against replicating B. burgdorferi with low MICs (≦0.2, ≦0.21, 0.76-1.57, ≦0.78 μg/mL, respectively). On the other hand, pyronin B and chaetochromin were less potent against growing B. burgdorferi with relatively high MICs (1.8-3.6, 2.74-5.47 μg/mL, respectively) but had excellent anti-persister activity.

TABLE 9
Comparison of the MIC values and anti-persister activity
of some compounds for B. burgdorferi

| | | Activity against |
|---|---|---|
| Antibiotics | MIC (μg/mL) | persisters^{a} |
| Doxycycline^{b} | ≦0.25 | 77% |
| Amoxicillin^{b} | ≦0.25 | 77% |
| Daptomycin^{b} | 12.5-25 | 18% |
| Daunomycin 3-oxime | ≦0.36 | 6% |
| Daunorubicin | ≦0.35 | 10% |
| NSC299187 | 3.26-6.52 | 13% |
| Pyronin B | 1.8-3.6 | 19% |
| Pyrromycin | 0.37-0.73 | 21% |
| Chaetochromin | 2.74-5.47 | 22% |
| Prodigiosin | ≦0.2 | 24% |
| Mitomycin | ≦0.21 | 25% |
| Nanaomycin | 0.76-1.57 | 26% |
| Dactinomycin | ≦0.78 | 30% |

^{a}Shown as residual viable cell percentage.
^{b}Cₘₐₓ values are derived from the published literature.

Comparison of Anti-Persister Activity at Low Drug Concentrations:

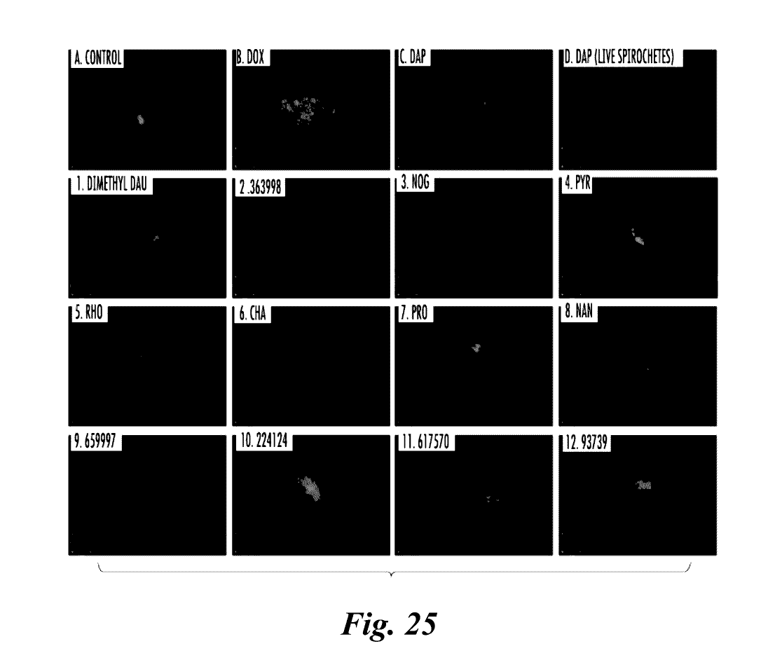

Although many highly effective hits were obtained from the NCI compound library with 50 μM compound screen, this drug concentration is likely too high for the in vivo experiments. Daptomycin at 50 μM has shown strong activity against stationary phase B. burgdorferi persisters in a previous study (Feng, Wang, Shi, et al., 2014), but it could not kill the microcolony form B. burgdorferi persisters at lower concentration such as 10 μg/mL (Feng et al. 2015, in press). To further compare the activity of hit compounds and daptomycin, the activity was tested against stationary phase B. burgdorferi persisters with 20 μM drug concentration (about 10 μg/mL for most compounds and 32 μg/mL for daptomycin). Most residual viable percentage of stationary phase B. burgdorferi increased with the decrease of drug concentration (Table 10, FIG. 25), but five anthracyclines, dimethyldaunomycin, NCS363998, nogalamycin, pyrromycin and Rhodomycin A, at 20 μM still showed as strong an activity against stationary phase B. burgdorferi persisters as 50 μM (Table 10, FIG. 25). Other non-anthracycline compounds showed relatively weaker activity than the daptomycin at 20 μM.

TABLE 10
Comparison of activity of some hit compounds at 20 μM
and 50 μM against stationary phase B. burgdorferi persisters^{a}

| | | Residual viable cells (%) | | |
|---|---|---|---|---|
| NSC^{d} | Compounds | 20 μM^{b} | 20 μM^{c} | 50 μM^{c} |
| | Control | 93% | 94% | 95% |
| | Amoxicillin | 77% | 77% | 77% |
| | Doxycycline | 76% | 77% | 77% |
| | Daptomycin | 32% | 25% | 18% |
| 258812 | Dimethyldaunomycin | 0% | 10% | 10% |
| | hydrochloride | | | |
| 363998 | Anthracene-9,10-dione, 1,5-bis[3- | 22% | 14% | 13% |
| | [[(2-hydroxyethyl)amino] | | | |
| | propyl]amino]-9,10-dihydro-, | | | |
| | dihydrochloride | | | |
| 70845 | Nogalamycin | 3% | 15% | 15% |
| 267229 | Pyrromycin | 6% | 20% | 21% |
| 136044 | Rhodomycin A | 5% | 21% | 21% |
| 345647 | Chaetochromin | 31% | 33% | 22% |
| 47147 | Prodigiosin | 0% | 45% | 24% |
| 267461 | Nanaomycin | 39% | 45% | 26% |
| 659997 | Naphthalene-1,4-dione, | 40% | 50% | 28% |
| | 2-chloro-5,8-dihydroxy-3-(2- | | | |
| | methoxyethoxy)- | | | |
| 224124 | (5,8-dihydroxy-1,4-dioxo- | 54% | 77% | 31% |
| | 1,4-dihydronaphthalene-2,3- | | | |
| | diyl)dimethanediyl dicarbamate | | | |
| 617570 | Benzoic acid, 2-hydroxy-, | 66% | 50% | 38% |
| | (2,6-pyridinediyldiethylidyne) | | | |
| | dihydrazide, nickel complex | | | |
| 93739 | 2-Methyl-4,4′-[(4-imino-2,5- | 0% | 77% | 43% |
| | cyclohexadien-1-ylidene)- | | | |
| | methylene]dianiline hydrochloride | | | |

^{a}Seven day old stationary phase B. burgdorferi culture was treated with drugs for 7 days.
^{b}Residual viable B. burgdorferi was calculated according to the regression equation and ratio of Green/Red fluorescence obtained by SYBR Green I/PI assay.
^{c}Residual viable B. burgdorferi was assayed by epifluorescence microscope counting.
^{d}The NSC number is a numeric identifier for substances submitted to the National Cancer Institute (NCI).

### Discussion

A number of interesting drug candidates have recently been identified that have excellent activity against non-replicating B. burgdorferi persisters from the FDA-approved drug library (Feng, Wang, Shi, et al., 2014). The goal of this study was to identify new chemical compounds that have high activity against B. burgdorferi persisters using the NCI compound library collection. From the 2526 compounds in three NCI compound libraries, 237 compounds were found to have higher activity against B. burgdorferi persisters than doxycycline or amoxicillin, from which the top 30 active hits were confirmed by microscopy rescreen. The use of the mechanistic compound library helped to identify the anthraquinone (anthracycline) class of drugs that have high activity against B. burgdorferi persisters. It is interesting to note that more than one third of the 30 most active compounds possess an anthraquinone (also called anthracenedione or dioxoanthracene) structure. The top 6 active compounds, daunomycin 3-oxime, dimethyldaunomycin, daunomycin (daunorubicin), NSC299187, NSC363998 and nogalamycin, are all anthraquinone derivatives, characterized by 3 aromatic rings linked together with benzoquinone in the center. Previously, the anti-persister activity of anthracycline antibiotic doxorubicin was noted (Feng, Wang, Shi, et al., 2014), but it was mistakenly excluded from the active drugs as it interfered with the SYBR Green I/PI staining. However, careful examination by microscopy confirmed the anti-persister activity by red colored anthraquinone drugs including doxorubicin. It is worth noting that not all red colored anthraquinone compounds have good anti-persister activity. For example, NCS156516 had weak anti-persister activity and showed 50% residual viable (green) cells (FIG. 24). Thus confirmation is needed in assessing compounds that have red color and have activity against B. burgdorferi persisters by careful microscopic examination, using low concentration of compounds and subculture studies.

It is of interest to note that the top six anthraquinone compounds with residual viable cells ranging from 6-15% seem to be even more active than daptomycin, which had 18% residual viable cells in the SYBR Green I/PI viability assay (Table 8). Nevertheless, since any single drug is unlikely to kill all bacterial populations and a drug combination using agents targeting both growing and non-growing persisters is required to more effectively kill heterogeneous bacterial populations in vitro and during persistent infection (Zhang, 2014), it would be necessary to evaluate the efficacy of an anthraquinone combination with current Lyme antibiotics against more resistant forms of B. burgdorferi persisters including microcolonies and biofilm-like growth. It has recently been shown that indeed daptomycin alone could not kill the aggregated form of B. burgdorferi persisters, such as microcolonies or biofilm-like structures, while daptomycin in combination with doxycycline and cefoperazone was able to completely eradicate the more resistant microcolonies or biofilm-like structures without any regrowth in subculture (Feng, et al., 2015, in press). Thus, further drug combinations and subculture studies are needed to confirm if the top six anthraquinone compounds are indeed more active than daptomycin against B. burgdorferi persisters in vitro and in vivo.

Anthraquinones are a class of naturally occurring phenolic compounds isolated from Streptomyces and have diverse medical uses including anti-cancer, antimalarial, and laxatives. Anthracycline antibiotics, such as daunomycin, nogalamycin, pyrromycin and rhodomycin A, were used in chemotherapy of some cancers, especially for several specific types of leukemia (Tan et al., 1967). It has been reported that anthracycline drugs have antibacterial activity against S. aureus, and the MICs of daunomycin and doxorubicin are 8-32 μg/mL and 0.12-0.5 μg/mL, respectively (Zhu et al., 2005). Daunomycin did not show bactericidal activity for Gram-negative bacteria Pseudomonas aeruginosa, Klebsiella pneumoniae and E. coli (Moody et al., 1978). This study is the first to demonstrate the activity of this class of compounds active against both growing and non-growing forms of Gram-negative B. burgdorferi. However, the mechanisms of action of this class of anthraquinone compounds against B. burgdorferi are unclear and remain to be determined. Anthracycline antibiotics could inhibit DNA and RNA synthesis by inserting into base pairs of the DNA/RNA strand (Mizuno et al., 1975). In addition, anthracycline antibiotics could stabilize the topoisomerase II complex and prevent dissociation of topoisomerase II from its nucleic acid substrate, leading to DNA damage and blocking DNA transcription and replication as well as producing reactive oxygen species, which could damage mitochondria and lead to cardiotoxicity as side effects (Jensen et al., 1993; Pommier et al., 2010). The sugar moiety of daunomycin plays a critical role in determining its anticancer activity (Zhu et al., 2005). In this study, it was found that anthracycline antibiotics with sugar structure and anthraquinone compounds without sugar structure (NCS299187 and NCS363998) all had good activity against B. burgdorferi persisters. These findings suggest that the mechanism of action of anthraquinone drugs may not be identical for its anti-cancer activity in eukaryotes and anti-persister activity in B. burgdorferi. Future studies are needed to identify the mechanism of action of anthracycline antibiotics against B. burgdorferi persisters, address the structure activity relationship of this class of compounds for B. burgdorferi persisters, and also to explore the possibility of utilizing the strong anti-persister activity of this class of compounds without untoward toxicity for the host cells.

Besides the anthracycline antibiotics, it was also found that some 1,4-naphthoquinones, such as nanaomycin, NCS659997 and NCS224124, showed high activity against stationary phase B. burgdorferi persisters. 1,4-naphthoquinone has an analogue molecular skeleton similar to anthraquinone. Nanaomycin may interfere with the function of the bacterial cell membrane and interact with the respiratory chain of bacteria (Hayashi et al., 1982), and such mode of action may explain its activity against B. burgdorferi persisters.

It was found that chaetochromin, a bis-naphtho-γ-pyrone produced by several species of chaetomium, also showed high activity against stationary phase B. burgdorferi. Bis-naphtho-γ-pyrones have a broad-range of biological activities such as inhibition of ATP synthesis in mitochondria, cells proliferation inhibition, triacylglycerol synthesis inhibition, and antimicrobial activity (Lu et al., 2014). Bis-naphtho-γ-pyrones were active against various bacteria such as S. aureus, E. coli and M. tuberculosis, with MIC values ranging from 2 to 50 μg/mL (Lu et al., 2014). Inhibition of ATP synthesis could explain the activity of bis-naphtho-γ-pyrone against B. burgdorferi persisters. Cephalochromin has been shown to inhibit fatty acid biosynthesis (Campbell and Cronan, 2001). It is possible that fatty acid synthesis might play a role in B. burgdorferi persister formation, and future studies are needed to confirm this possibility.

In this study, it was found that some antibiotic compounds, such as prodigiosin, mitomycin, and dactinomycin, had decent activity against B. burgdorferi persisters, though their activities (24-30% residual viable cells) were not as strong as daptomycin (18% residual viable cells) (Table 8). Prodigiosin is a secondary metabolite of Serratia marcescens and is well known to have antibacterial, antifungal, antiprotozoal, antimalarial, immunosuppressive and anticancer activities (Williamson et al., 2007). Mitomycin shows its activity as a DNA crosslinker through its aziridine functional group and crosslinks the complementary strands of the DNA double helix to cause the death of a bacterial cell (Szybalski and Iyer, 1964; Tomasz, 1995). The activity of mitomycin against B. burgdorferi persisters may also be due to its DNA crosslinking activity. Dactinomycin is a polypeptide antitumor antibiotic isolated from soil bacteria Streptomyces (Hollstein, 1974) and is known to bind DNA and interfere with DNA replication (Hollstein, 1974), and also inhibit RNA transcription (Sobell, 1985).

In addition, some unstudied compounds, such as NSC343783 and NCS311153, were found to be effective against stationary phase B. burgdorferi persisters to a varying extent. These newfound interesting compounds have more anti-persister activity than current Lyme antibiotics and may be explored in the future as leads for further drug development and mechanism study for bacterial persistence.

In summary, the anthracycline class of compounds and antibiotics along with some other compounds, including prodigiosin, mitomycin, nanaomycin and dactinomycin, have been identified as having excellent activity against B. burgdorferi persisters. The structure activity relationship and mechanisms of action of the anthracycline/anthraquinone class of compounds against B. burgdorferi persisters should be addressed in future studies. Drug combination studies with the anthracycline/anthraquinone class of compounds and the current Lyme antibiotics to eradicate B. burgdorferi persisters in vitro and in animal models should be of value for improved treatment of Lyme disease.

### Example 5

### An Ultra Rapid Antibiotic Susceptibility Test in 30 Minutes to 2 Hours by SYBR Green/PI Assay

### Introduction

Pathogens isolated from clinical specimens undergo antimicrobial drug susceptibility testing to help clinicians provide better antimicrobial therapy. It is important to identify drugs that can be used most effectively to cure the patient especially those who are infected with antibiotic resistant pathogens. For patients who are infected with bacteria that can cause life-threatening infections (Staphylococcus, Neisseria, Acinetobacter, Pseudomonas, Stenotrophomonas, Haemophilus, Escherichia), it is crucial that antimicrobial drug susceptibility tests be performed in a quick manner. Currently, the common drug susceptibility tests all rely on growth of the organisms and take at least one day for non-fastidious organisms (CLSI, 2016) and can be up to 1 month for fastidious organisms (CLSI, 2011) before any information can be determined about the pathogen, risking patients' lives during the waiting period for antibiotic susceptibility testing results, with potential worsened disease outcome and spread of antibiotic resistant organisms. Here, a rapid antibiotic susceptibility test is disclosed that does not rely on growth of the organism, but rather rely on detecting the viability of the organisms after drug exposure in a short time, which have been found to produce robust results in only 30 minutes—two hours for fast growing organisms such as S. aureus and E. coli and in 16 hrs for slow growing organisms, such as M. tuberculosis.

Antimicrobial activity is measured by determining the minimum inhibitory concentration (MIC), the lowest amount of antibiotics that is needed to inhibit the growth of the organism. Hence, the current tests are lengthy in time and depend on the growth of the pathogen. The disk diffusion (Kirby-Bauer Disk Diffusion) test consists of inoculating an agar plate with a known organism and then adding antibiotics (with concentrations recommended by the Clinical and Laboratory Standards Institute) onto filter-paper disks that are then placed onto the surface of the agar (CLSI, 2016). During incubation, which can take up to at least 16 hours (one day) even for fast growing organisms, the antibiotic will diffuse from the disk and into the agar. A zone of inhibition will form once the bacteria are grown up and the diameter of the zone of inhibition will be measured and used to extrapolate susceptibility categories based on zone size. The standards for comparison are provided by the Clinical and Laboratory Standards Institute (CLSI) (CLSI, 2016; CLSI, 2011). Similarly, the MIC determination procedure in liquid culture broth also takes a long time and is dependent on how fast the organism grows. Tubes or wells containing serial dilutions of drugs are inoculated with bacteria and growth in the presence of drug is observed by the turbidity of the culture, which also takes at least 16 hours for fast growing organisms to several weeks for slow growing organisms.

To expedite the process of antimicrobial susceptibility testing, the presently disclosed methods use SYBR Green/Propidium Iodide (PI) staining viability assay, which was initially developed for quantitating the viability of Borrelia burgdorferi (Feng, et al., 2014) and has since been adapted for viability assessment and antibiotic susceptibility testing for different bacteria as described herein. The SYBR Green I dye is commonly used in molecular biology to stain nucleic acids and was used for viability assessment for some bacteria by flow cytometry, (Barbesti, et al., 2000) but has not been commonly used for bacterial viability assessment, until it was used for drug susceptibility testing for B. burgdorferi (Feng, et al., 2014). SYBR Green I is a permeable dye that stains all cells green and PI is an impermeant dye that stains only dead or damaged cells with a compromised cell membrane (Feng, et al., 2014). Hence, the live cells will be stained green by SYBR Green I while dead cells stained red by PI. One rapidly determines the viability of a bacterial population based on the ratio of green cells and red cells as measured by fluorescence microscopy or fluorescence microplate readers. Because the SYBR Green/PI stain conveniently measures the presence of live and dead cells by green versus red fluorescence ratio, this assay is not growth-dependent and eliminates a bottleneck in current antibiotic susceptibility tests—which depend on the lag period waiting for bacteria to grow as well as bacterial growth to certain numbers for detection by sensors in automated systems or visual measurements. It is important to note that aside from producing rapid results, the SYBR Green/PI staining assay also has other advantages. Compared to other colorimetric viability assays such as MTT and Alamar Blue, the SYBR Green/PI assay is more rapid and has lower detection limit, background, rate of error, is extremely cost-effective, and can be used in a high-throughput manner in 96-well or 384 well plates. In addition, the SYBR Green I/PI assay has been used for drug screens with compound libraries (Feng, et al., 2014; Feng, et al., Emerg Microbes Infect, 2014).

As provided herein, the SYBR Green/PI stain can successfully stain both live and dead populations of five different pathogens, Gram-positive Staphylococcus aureus (USA300), Gram-negative Escherichia coli (W3110), Klebsiella pneumoniae (Isolate 7), Acinetobacter baumanii and acid-fast Mycobacterium tuberculosis (H37Ra), as representatives for other bacteria, to demonstrate the applicability of this method for antibiotic susceptibility testing of different bacterial species. The SYBR Green/PI assay can detect the MIC of antibiotics that is concordant with results from the traditional broth dilution method and also, detect the amount of drug tolerant persisters that is concordant with results from traditional cell forming unit (CFU) enumeration. Further, the SYBR Green/PI assay also has been optimized to assign different strains of S. aureus, E. coli, and K. pneumoniae with different antibiotic sensitivity and resistance profiles into their respective drug susceptibility categories (as defined by CLSI standards) under 2 hours.

### Methods

### Culture Media, Chemicals, and Antibiotics

Staphylococcus aureus, Escherichia coli, Klebsiella pneumoniae, Acinetobacter baumanii cultures were cultivated in tryptic soy broth (TSB) and tryptic soy agar (TSA) (Becton Dickinson) and Mycobacterium tuberculosis cultures were cultivated in 7H9 medium at 37° C. with the appropriate antibiotics. Staphylococcus aureus strains USA300, CA-409, NY-315, CA-127, Escherichia coli strains W3110, UTI89, CFT073, and KTE181, Klebsiella pneumoniae (Isolate 7), Acinetobacter baumanii isolate and M. tuberculosis H37Ra, were obtained from ATCC (Manassas, Va.). Stock solutions of the antibiotics ampicillin, chloramphenicol, gentamicin, erythromycin, trimethoprim, ciprofloxacin, streptomycin, ceftriaxone, and cefotaxime (Sigma-Aldrich Co.) were prepared and sterilized through filtration or autoclaved, if necessary, and used at indicated concentrations. Antibiotic susceptibility assays for different bacteria were performed using Mueller-Hinton broth and agar per CLSI methods as described below.

### Conventional Antibiotic Susceptibility Tests

The Kirby-Bauer disk diffusion assay was used to determine the susceptibility of the various strains to antibiotics. Instructions and also interpretation of zone diameters were made using the established standards as listed in the Clinical and Laboratory Standards Institute (CLSI). Briefly, bacterial cultures were adjusted to a 0.5 McFarland turbidity standard and spread onto Mueller-Hinton agar. Paper Disks were impregnated onto the agar and the antibiotics at concentrations recommended by CLSI were pipetted onto each blank disk and allowed to air dry for 10 minutes. The plates were incubated at 37° C. overnight before the zone of inhibition was measured in millimeters. The protocol used to test for antibiotic susceptibility using a broth dilution method was based on the recommendations of CLSI. Briefly, bacterial cultures were adjusted to a 0.5 McFarland turbidity standard in Mueller-Hinton broth and drug concentrations of 2-fold dilutions beginning with 32 μg/mL down to 1 g/mL were tested. The minimum inhibitory concentration was defined as the lowest drug concentration that completely inhibited visible bacterial growth.

### SYBR Green I/PI Assay

SYBR Green I (10,000× stock, Invitrogen) was mixed with propidium iodide (20 mM, Sigma) in distilled H₂O. The staining dye for Staphylococcus aureus, Escherichia coli, Acinetobacter baumanii, and Mycobacterium tuberculosis were made by mixing SYBR Green I to propidium iodide (1:3) in 100 μl distilled H₂O. The staining dye for Klebsiella pneumoniae was made by mixing SYBR Green I to propidium iodide (3:1) in 100 μl distilled H₂O. The SYBR Green/PI staining mix (10 μl) was added to each 100 μl of each sample. The sample was vortexed and incubated at room temperature in the dark for 20 minutes. The green and red fluorescence intensity was detected using a Synergy H1 microplate reader by Bio Tek Instruments (VT, USA) at excitation wavelength of 485 nm, 538 nm (green emission), and 612 nm (red emission). The percentage of live cells in each sample was determined by a regression equation generated by a standard curve. To generate a standard curve, different proportions of live and isopropyl alcohol killed cells were made. The staining mixture was added to each sample and the green/red fluorescent ratios were measured as described above. The regression equation was generated using the least-square fitting analysis. Specimens were examined on the Keyence BZ-X710 Fluorescence Microscope and images were recorded and processed using BZ-X Analyzer provided by Keyence (Osaka, Japan).

### SYBR Green/PI Viability Staining to Determine Antimicrobial Susceptibility to Antibiotics

Stationary phase cultures were diluted to indicated concentrations before treatment with varying concentrations of antibiotics (0 μg/mL, 5 μg/mL, 30 μg/mL, 50 μg/mL, and 100 μg/mL). After 1.5 hr of incubation at 37° C., the cultures were stained with SYBR Green/PI and the percentage of viable cells was determined as described above.

### Persister Assay

Stationary phase cultures of E. coli UTI89 were treated with ofloxacin (5 μg/mL) for 4 hours to enrich for persisters, followed by drug exposure with different antibiotics including ampicillin (100 μg/mL), ofloxacin (5 μg/mL), polymycin B (10 μg/mL) and gentamicin (20 μg/mL). The bacterial viability was determined by CFU enumeration and SYBR Green/PI assay as described above.

### Results

### SYBR Green/PI Assay can be Used for Assessing the Viability of Various Bacterial Pathogens

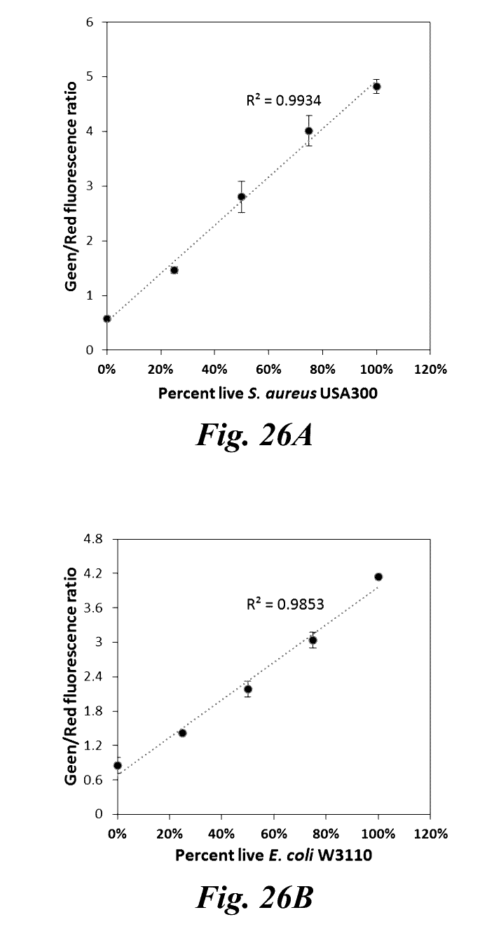

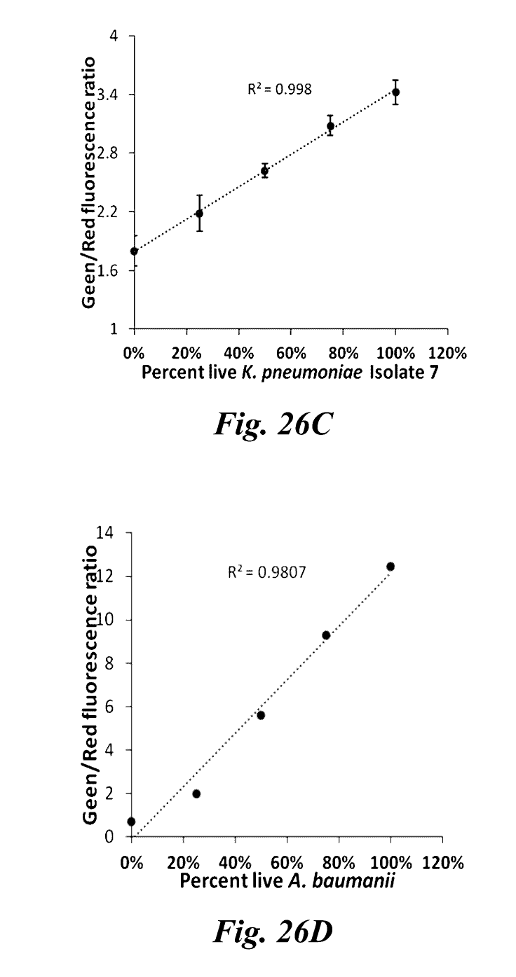

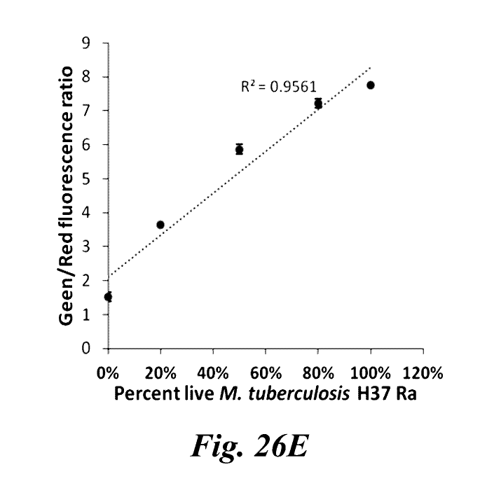

To develop a rapid test to evaluate antimicrobial susceptibility in the clinic, it is important to ensure that SYBR Green/PI staining can assay the viability for a range of organisms. To validate that the SYBR Green/PI stain can accurately measure the viability of different bacteria, the SYBR Green/PI assay was applied to generate a standard curve for Gram-positive bacteria using S. aureus as an example, Gram-negative bacteria using E. coli as an example, and mycobacteria using M. tuberculosis as an example. The SYBR Green/PI assay, which was developed for assessing the viability of Borrelia burgdorferi (Feng, et al., 2014), was adapted. First, the bacteria were grown in their normal culture media, and to generate dead cells, the bacteria were killed by treating the cells with 70% isopropyl alcohol for one hour. Mixtures were then made of live and dead cells in known proportions. After staining the cells with SYBR Green/PI, the green and red fluorescence intensities of the different samples were measured using a fluorescence microplate reader (BioTek Synergy HT) and a standard curve was generated. A linear relationship between the green/red fluorescence ratio and the percentages of live S. aureus, E. coli, Klebsiella pneumoniae, Acinetobacter baumanii, and M. tuberculosis with R² values of 0.9934, 0.9853, 0.9980, 0.9807, and 0.9561, respectively, were generated (FIG. 26A, FIG. 26B, FIG. 26C, FIG. 26D, and FIG. 26E).

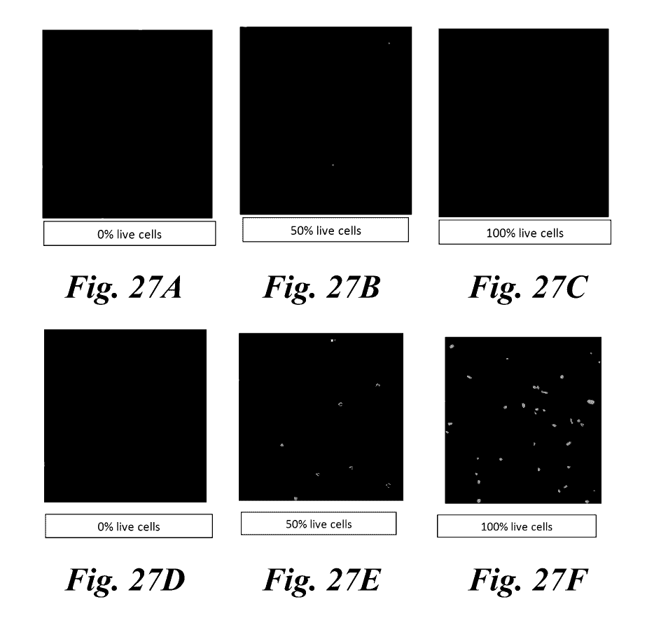

The results indicate that SYBR Green/PI can successfully act as a viability stain for S. aureus, E. coli, K. pneumoniae, A. baumanii, and M. tuberculosis. Additionally, staining of varying viable proportions (0%, 50%, and 100%) of Gram-positive S. aureus and Gram-negative K. pneumoniae samples with SYBR Green/PI revealed that there is a good correlation between the amount of viable cells in the samples from fluorescence microscopy imaging and the actual known viable percentages (FIG. 27A, FIG. 27B, FIG. 27C, FIG. 27D, FIG. 27E, and FIG. 27F).

### Validation of the SYBR Green/PI Assay for Antibiotic Susceptibility Testing

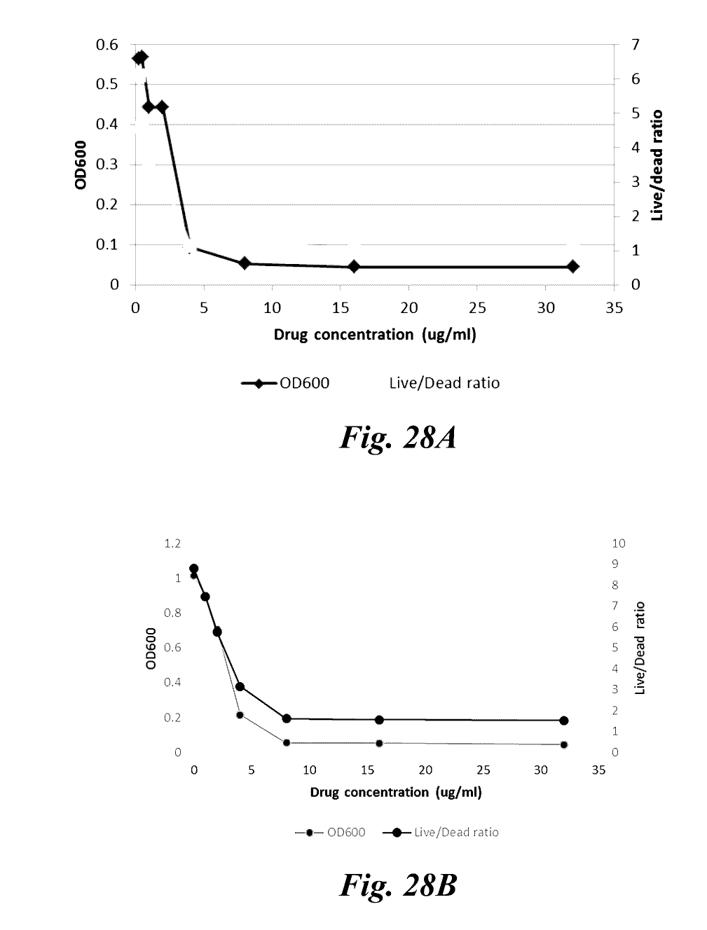

To assess if the SYBR Green/PI assay could be used for antimicrobial susceptibility testing, the SYBR Green/PI assay was tested to detect the MIC of chloramphenicol and ampicillin for S. aureus and E. coli, respectively. Following the protocols for antibiotic susceptibility testing (AST) method based on measuring the turbidity of bacterial growth as described by the CLSI (CLSI, 2016), the bacterial cultures were subjected to 2-fold dilutions of antibiotics ranging from (0 μg/mL to 32 μg/mL). After overnight incubation, no signs of turbidity was detected in the sample with 4 μg/mL of chloramphenicol for S. aureus (FIG. 28A) and 8 μg/mL of ampicillin for E. coli (FIG. 28B). No signs of turbidity were determined by OD600 readings of approximately 0.5, the read-out of the background signal. Similarly, the decrease and subsequent plateau of the live/dead ratio from SYBR Green/PI staining also reveals the same MIC for both bacteria and the respective drug, suggesting that SYBR Green/PI can be used to determine antibiotic susceptibility results that are similar to the currently accepted standard culture based AST method.

### Development of a Rapid Drug Susceptibility Test Using SYBR Green/PI Assay

Because resistant strains are tougher to kill than susceptible strains, SYBR Green/PI assay will be able to distinguish resistant and susceptible categories based on the amount of residual viable cells in the culture after drug treatment. Since the SYBR Green/PI assay detects viability and killing (Feng, et al., 2014), this assay will eliminate the lag time needed for growth to occur, and produce antimicrobial susceptibility results in a shorter time than the traditional growth based methods.

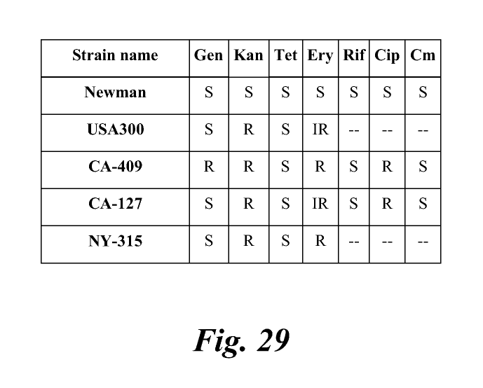

To develop and validate the usage of SYBR Green/PI staining as a means for rapid antimicrobial testing, it had to be ensured that the assay can determine results that are in concordance with the current standards provided by CLSI. Thus, various S. aureus (Newman, USA300, CA-409, CA-127, NY-315) strains from ATCC were obtained and tested their susceptibility categories against bacteriostatic and bactericidal drugs based on the traditional Kirby-Bauer disk diffusion assay (FIG. 29). Next, after diluting overnight cultures of bacteria 1:500 (approximately 10⁶ CFU/mL and OD600=0.05), treatment of varying concentrations of antibiotics (0 μg/mL, 5 μg/mL, 30 μg/mL, 50 μg/mL, and 100 μg/mL) were added. After incubation with antibiotics for 1.5 hours, SYBR Green/PI staining was done to determine the amount of viable cells remaining after antibiotic treatment.

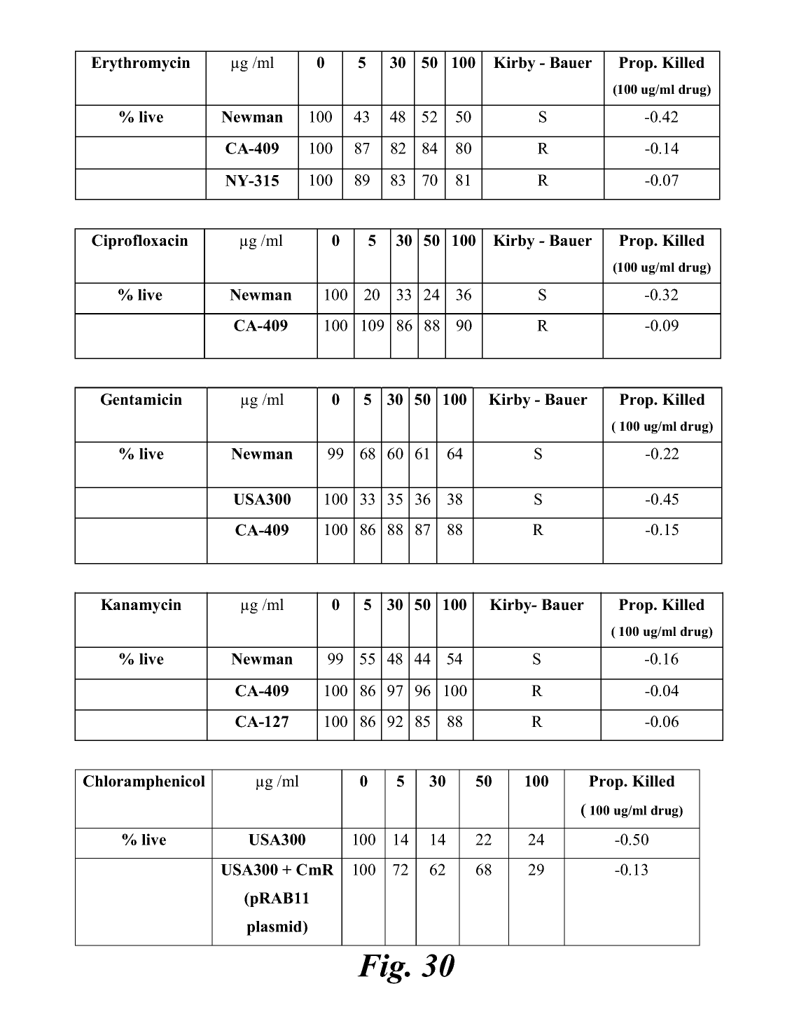

For S. aureus strains CA-409, NY-315, and CA-127, the percentages of live cells across the increasing drug concentration gradients of erythromycin, ciprofloxacin, gentamicin, and/or kanamycin determined by the green/red fluorescence ratio from SYBR Green/PI staining were higher than pan-susceptible Newman strain, a laboratory sensitive control strain, indicating that strains CA-409, NY-315, and CA-127 are resistant to the respective drugs (FIG. 30). Similarly, the SYBR Green/PI staining also was able to detect the difference between strain USA300 and USA300 inserted with a chloramphenicol resistance (pRAB11) plasmid (FIG. 30). On the other hand, the strain USA300 showed equal to or a lower percentage of viable cells than Newman across the increasing concentrations of gentamicin, suggesting that USA300 is susceptible to gentamicin (FIG. 30). The antibiotic susceptibility determined by the SYBR Green/PI stain matched completely with the susceptibility results of the traditional Kirby-Bauer disk diffusion test but was performed in only 2 hours (FIG. 29 and FIG. 30).

To establish a quantitative cut-off to distinguish resistant and susceptible strains, a mathematical formula was used to determine the proportion of cells killed based on the green and red fluorescence values from microtiter plate readings. The proportion of cells killed is calculated by the formula:

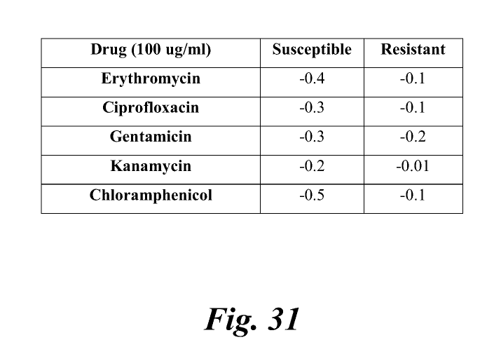

(LD_{treated}−LD_{untreated})/LD_{untreated}

where LD is equal to the ratio of live (green fluorescence) and dead (red fluorescence) cells. Upon calculating the respective killed proportions for all susceptible and resistant strains tested (FIG. 30), cut-offs distinguishing resistant and susceptible strains for the different antibiotics tested were established by averaging all the values (i.e., proportion of killed cells) from the respective susceptibility categories (FIG. 31). The cut-off for S. aureus strains susceptible and resistant to erythromycin (100 μg/mL) is −0.4 and −0.1, respectively; the cut-off for ciprofloxacin (100 μg/mL) susceptibility and resistance is −0.3 and −0.1, respectively; the cut-off for gentamicin (100 μg/mL) susceptibility and resistance is −0.3 and −0.2, respectively; the cut-off for kanamycin (100 μg/mL) susceptibility and resistance is −0.2 and −0.01, respectively; the cut-off for chloramphenicol (100 μg/mL) susceptibility and resistance is −0.5 and −0.1, respectively (FIG. 31).

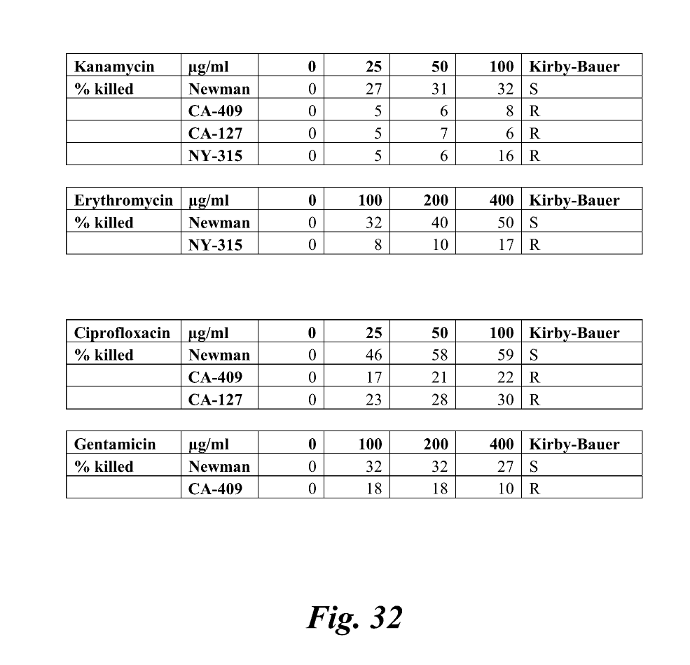

To evaluate if the detection time of the presently disclosed SYBR Green/PI AST could be accelerated to under 2 hours, the inoculum size of the bacteria was increased before drug treatment. As opposed to diluting overnight cultures of bacteria to TSB medium 1:500 in the rapid 1.5 hr assay, overnight cultures were diluted to TSB 1:25. The data suggests that increasing the inoculum size of the culture can decrease the time of the AST down to 30 minutes. By generating a cut-off of 20% in proportion of killed cells, the presently disclosed AST can distinguish sensitive and resistant strains as determined by traditional Kirby-Bauer methods. After 30 minutes, kanamycin (25 μg/mL) killed only 5% of resistant strains CA-409, CA-127, NY-315 as opposed to 27% killing of the sensitive Newman strain. After erythromycin (400 μg/mL) treatment, resistant strains NY-315 had 17% killed as opposed to 50% killing in the sensitive Newman strain. Ciprofloxacin (50 μg/mL) killed 21% and 28% in resistant strains CA-409 and CA-127, respectively, while 58% was killed in sensitive Newman strain. Lastly, gentamicin (400 μg/mL) killed 10% in the resistant strain CA-409 and 27% in the sensitive Newman strain (FIG. 32).

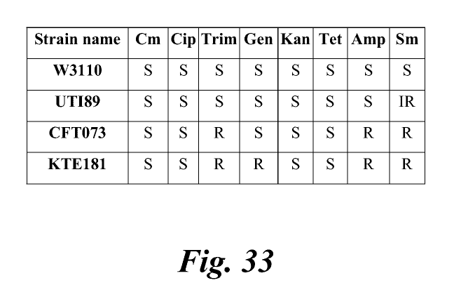

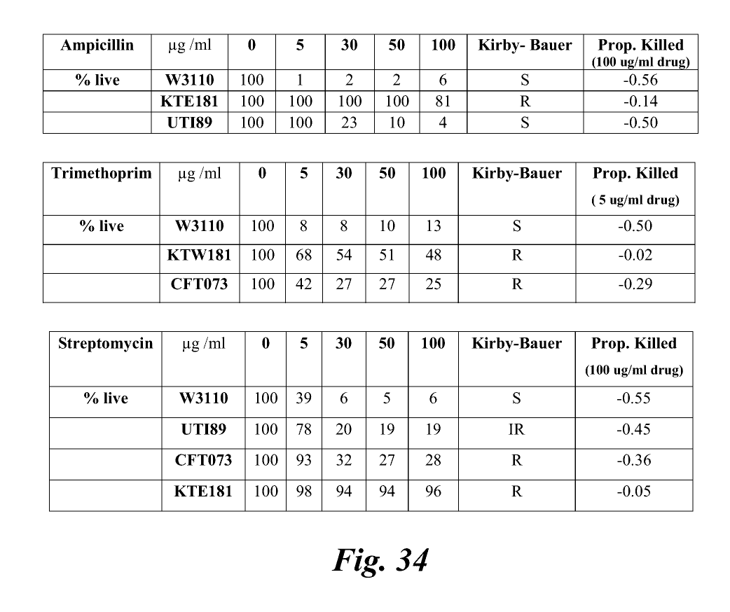

To ensure that the SYBR Green/PI assay can be applied to different bacterial pathogens, the antimicrobial susceptibility testing was performed for various E. coli strains including K12-strain W3110, and uropathogenic clinical strains UTI89, CFT073, KTE181 and tested their susceptibility categories against bactericidal and bacteriostatic drugs using the traditional Kirby-Bauer disk diffusion assay as a reference control method (FIG. 33). After performing the protocol for rapid antimicrobial susceptibility testing as mentioned above, the strains KTE181 and CFT073 showed a higher percentage of live cells throughout increasing concentrations of ampicillin, trimethoprim, and streptomycin compared to W3110, the laboratory strain used as a susceptible control strain, suggesting that KTE181 is resistant to ampicillin, trimethoprim, and streptomycin (FIG. 34). At increasing concentrations of ampicillin, the strain UT189 have an amount of live cells that was comparable to W3110, which indicates that KTE181 is sensitive to ampicillin (FIG. 34). Not only was the SYBR Green/PI viability assay able to distinguish between susceptible and resistant strains that correlate with the traditional Kirby-Bauer disk diffusion method, the assay also distinguished intermediate resistance as exemplified by findings for UTI89 in streptomycin testing. For streptomycin testing, strains KTE181 and CFT073 showed higher percentages of live cells across all drug concentrations compared to the susceptible control strain W3110, suggesting their resistance. However, while strain UTI89 showed higher percentages of live cells in all drug concentrations compared to W3110, the percentages of live cells were lower than the resistant strains, suggesting that UTI89 has intermediate resistance against streptomycin (FIG. 33 and FIG. 34).

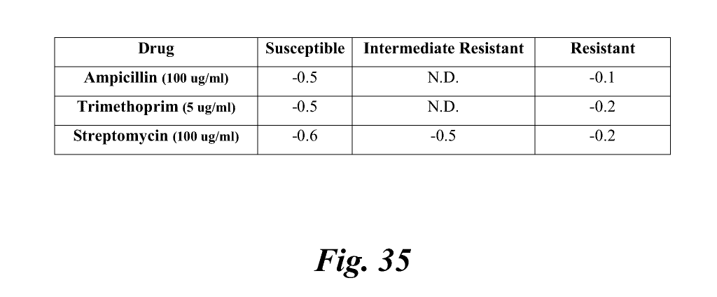

The same formula as described above was used to establish cut-offs for susceptibility and resistance for E. coli AST. The cut-off for E. coli strains susceptible and resistant to ampicillin (100 μg/mL) is −0.5 and -0.1, respectively; the cut-off for trimethoprim (5 μg/mL) susceptibility and resistance is −0.5 and -0.2, respectively; the cut-off for streptomycin (100 μg/mL) susceptibility, intermediate resistance and resistance is −0.6, −0.5 and −0.1, respectively (FIG. 34 and FIG. 35).

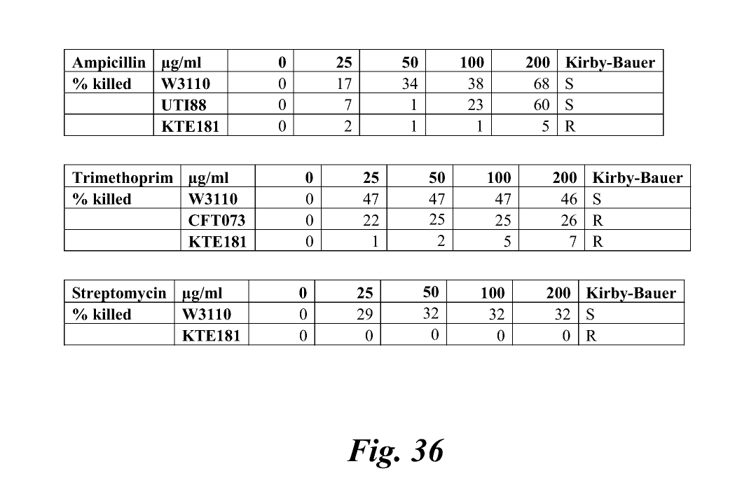

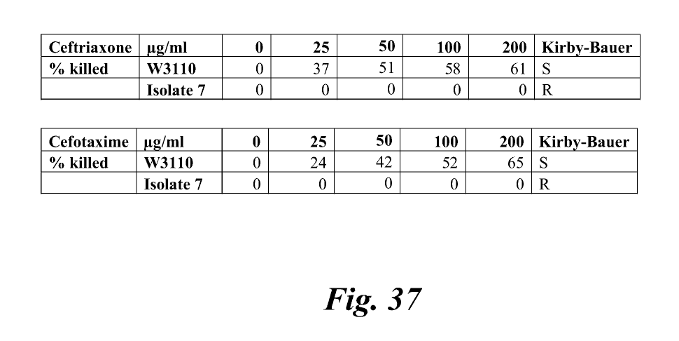

Similar to S. aureus, whether AST can be performed in 30 minutes in E. coli was tested, as well. Using the same conditions of diluting overnight cultures to fresh TSB media 1:25, similar AST results as traditional Kirby-Bauer using the SYBR Green/PI assay were observed. Upon ampicillin treatment (100 μg/mL), sensitive strains W3110 and UTI89 were killed 38% and 23%, respectively, while resistant strain KTE181 was killed only 1%. Trimethoprim treatment (25 μg/mL) killed 47% of sensitive strain W3110 but 22% and 1% of resistant strains CFT073 and KTE181, respectively. Streptomycin treatment (25 μg/mL) killed 29% of sensitive strain W3110 but 0% in resistant strain KTE181. Similarly, the presently disclosed assay was tested in another Gram-negative organism, K. pneumoniae. Upon ceftriaxone and cefotaxime (25 μg/mL) treatment, sensitive strain W3110 was killed 37% and 24%, but the resistant K. pneumoniae strain was not killed at all. The presently disclosed AST produced findings in concordance with results from the Kirby-Bauer disk diffusion test for both E. coli and K. pneumoniae. (FIG. 36 and FIG. 37).

### Validation of the SYBR Green/PI Assay in Quantitating Persister Numbers

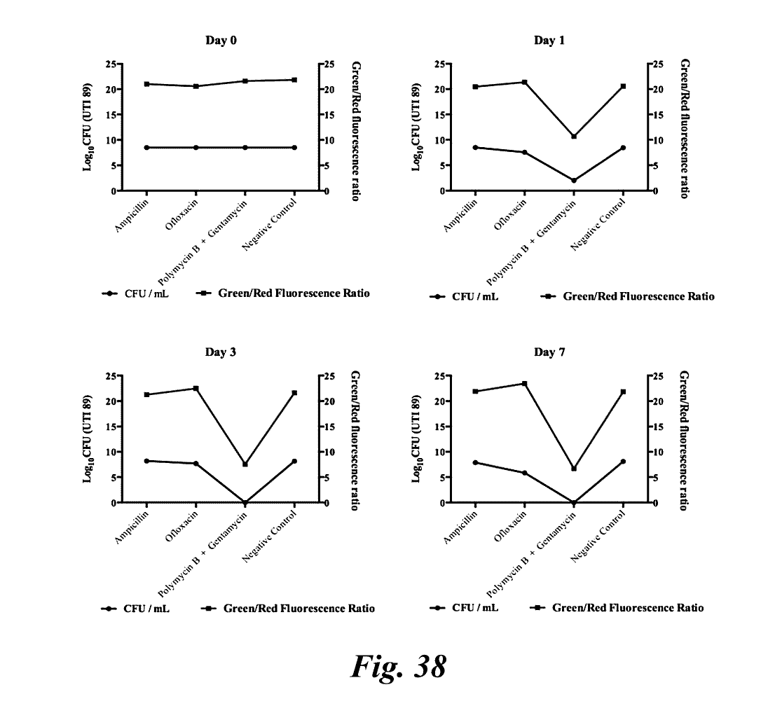

While antibiotic resistance is a major public health problem, antibiotic persistence mediated by persister cells play a significant role in causing recurrent and relapsing infections (Zhang, 2014). Research and development in novel drug therapies to eradicate persistent infections such as persistent Lyme Disease (Feng, et al., Emerg Microbes Infect, 2014), biofilm infections from indwelling devices, endocarditis & osteomyelitis caused by S. aureus (Niu, et al., 2015) and urinary tract infections caused by E. coli (Niu, et al., Antibiotics, 2015) are of utmost importance. Currently, researchers are depending on serial dilutions to enumerate colonies forming on agar plates, which is also dependent on bacterial growth and hence, time-consuming. To test if SYBR Green/PI can be used to rapidly detect the presence of persisters in different drug treatments, persister-enriched cultures of E. coli were exposed to single drug treatment of ampicillin and ofloxacin, and a drug combination of gentamicin and polymycin B. Starting at Day 3, the decrease in the level of persisters after exposure with the combination of polymycin B and gentamicin can be detected by both CFU enumeration and also green/red fluorescence ratio from SYBR Green/PI staining. In the samples with a single drug treatment (ampicillin and ofloxacin) and the no drug treated sample (negative control), both CFU enumeration and SYBR Green/PI staining revealed that the amount of persisters stayed constant throughout the course of 7 days (FIG. 38). The presently disclosed findings suggest that the SYBR Green/PI assay can also be used to perform high-throughput drug screens as was demonstrated with Borrelia burdorferi (Feng, et al., Emerg Microbes Infect, 2014; Feng, et al., 2015; Feng, et al., 2016) to search for better therapies for recurrent infections by other bacterial pathogens.

### Discussion

The presently disclosed subject matter provides an ultra-rapid antibiotic susceptibility testing (AST) method using SYBR Green I/PI viability assay that is growth independent and can be performed in hours rather than days and weeks for the current AST. The current conventional AST relies on the growth of the test organism in the presence of the antibiotic, which can be time consuming depending the growth rate of the test organism, ranging from days with fast growing bacteria to weeks for slow growing bacteria. The presently disclosed subject matter demonstrates that by using an optimized SYBR Green I/PI assay, the AST for representative Gram-positive, Gram-negative and acid fast bacteria using S. aureus, E. coli could be determined in less than 2 hrs, and for and for slow growing mycobacteria, M. tuberculosis, in 16 hrs rather than days or weeks as current tests. The presently method takes advantage of the rapid SYBR Green I/PI viability assay which was developed initially for rapid assessment of viability of B. burgdorferi (Feng, et al., 2014) and was used for high throughput drug screens against B. burgdorferi persisters (Feng, et al., Emerg Microbes Infect, 2014; Feng, et al., 2015; Feng, et al., 2016). Here, this SYBR Green I/PI viability assay was adapted and found to work very well for AST for different bacteria (FIG. 29, FIG. 30, FIG. 31, FIG. 32, FIG. 33, and FIG. 34).

Antibiotics with different modes of action, including cell wall inhibitor ampicillin, protein synthesis inhibitor streptomycin, folate synthesis inhibitor sulfa drug trimethoprim, DNA gyrase inhibitors ciprofloxacin (quinolones) were tested in the SYBR Green I/PI assay and they all worked well and could determine the AST results in less than 2 hr for fast growing bacteria like E. coli and S. aureus. It is worth noting that the SYBR Green I/PI assay not only works for bactericidal antibiotics such as ampicillin, streptomycin, ciprofloxacin, gentamicin, and kanamycin and also bacteriostatic drugs such as sulfa drug trimethoprim, erythromycin, and chloramphenicol (FIG. 30 and FIG. 33).

The presently disclosed subject matter used liquid stationary phase cultures as inocula, which is more compatible with the current blood culture system used commonly in the clinic lab setting. Previous studies have shown that flow cytometry can be used for AST testing for M. tuberculosis in 24 hr (Hughes, et al., 2005). However, the test still relies on detecting of growth by fluorescence diacetate, and the most significant drawbacks are the requirement for expensive instrumentation and lack of throughput by the flow cytometry method. Because of these shortcomings, the flow cytometry method has not been adopted by clinical or field settings. Although a luciferase phage system has been developed for rapid AST for RIF for mycobacteria in 2 days (Wilson, et al., 1997; Jacobs, et al., 1993), it has contamination issues that limit its use in field settings (Mole, et al., 2007).

Advantages of the new SYBR Green I/PI assay include: rapid AST results in less than 2 hrs for fast growers and 16 hrs for slow growers, low cost using plate reader without use of expensive instrumentation (mass spectrometry and flow cytometry), and high throughput capability in 96-well or 384 well plate format.

### Example 6

### A Rapid Pyrazinamide Susceptibility Test in 16 Hours by SYBR Green/PI Assay Abstract

A rapid PZA drug susceptibility testing (DST) method was developed based on a modified SYBR Green I/PI assay for viability assessment of M. tuberculosis. Using this SYBR Green/PI assay, PZA resistance in resistant mutants could clearly be distinguished from PZA susceptible control strain. This SYBR Green/PI assay has allowed the time required for DST results to be reduced from weeks to less than 1 day. This is made possible using the SYBR Green I/PI viability assay by changing the concept of DST through converting a conventional time-consuming growth/culture-dependent DST to a viability based and growth/culture-independent DST. The presently disclosed subject matter demonstrates the feasibility and reliability of adapting the SYBR Green I/PI assay for rapidly identifying PZA resistance in M. tuberculosis. The SYBR Green I/PI assay overcomes not only the inefficiency of classic microbiology growth/culture based DST relying on growth of slow growing M. tuberculosis (which takes several weeks), but also the limitations of molecular testing methods (with compromised sensitivity compared with phenotype based DST as mutations are not all covered by molecular tests). The SYBR Green I/PI assay can be developed as a convenient, accurate and quantitative test for rapid DST for all TB drugs, with capability of high throughput, thus providing timely guidance on clinical treatment of drug resistant TB, with high efficiency and low cost that can be easily adapted for field application.

### BACKGROUND

Tuberculosis (TB) remains a leading infectious killer worldwide, with 9 million new TB cases and 1.5 million deaths annually (WHO, 2008). Drug-resistant tuberculosis (TB), especially multidrug-resistant TB (MDR-TB) (with bacillary resistance to at least isoniazid and rifampicin) and extensively drug-resistant TB (XDR-TB) poses an increasing challenge for TB control (WHO, 2008). The standard 6 month TB therapy recommended by WHO for treatment of drug susceptible TB consists of 2 months of daily treatment with isoniazid (INH), rifampicin (RIF), pyrazinamide (PZA) and ethambutol, followed by 4 months of daily or intermittent treatment with INH and RIF (WHO, 2008). The lengthy therapy makes patient compliance difficult and increases the risk of development of drug resistant TB.

PZA plays a unique role in shortening of the treatment from previously 9-12 months to 6 months and is used for treating both drug susceptible and drug resistant TB (Mitchison, 1985). The powerful sterilizing activity of PZA is due to its ability to kill a population of persister tubercle bacilli that are not killed by other TB drugs (Mitchison, 1985). However, PZA can have potential hepatotoxicity (Chang, et al., 2008), and should be avoided when there is bacillary resistance demonstrated. In a number of available studies, PZA resistance in MDR-TB ranges from 10% in Papua New Guinea (Simpson, et al., 2011), 25% in Turkey (Senol, et al., 2008), 49% in Thailand (Jonmalung, et al., 2010), to 52% in South Africa (Louw, et al., 2006), 53% in Japan (Ando, et al., 2010), 76.6% in Pakistan (Rao, et al., 2010); 85% in India (Shenau, et al., 2009). Bacillary resistance to PZA is even higher in XDR-TB cases (Velayati, et al., 2009). In view of the potential importance of PZA resistance on MDR-TB treatment outcome (Migliori, et al., 2007), it is critical to determine the prevalence of PZA resistance and susceptibility in MDR-TB strains.

However, the PZA drug susceptibility testing (DST) methods, such as Lowenstein-Jensen medium and 7H10/11 agar at pH 5.5, BACTEC 460 and MGIT 960 or BacT/ALERT systems at pH 6.0 (Aragon, et al., 2007), are not reliable and are subject to frequent false resistance problem (Zhang, et al., 2003; Hewlett, et al., 1995; Chedore, et al., 2010). In a recent study, the MGIT960 PZA susceptibility testing method showed even less reliable results than the discontinued radioactive BACTEC 460 method causing more false resistance problem presumably due to higher inoculum size used in the MGIT method (Chedore, et al., 2010). These phenotype based PZA DST methods rely on growth of slow growing M. tuberculosis, which can take a lengthy period of 2-6 weeks to obtain the DST results.

Since mutation in the pncA gene encoding pyrazinamidase/nicotinamidase (Scorpio, et al., 1996) is the major mechanism for PZA resistance in M. tuberculosis (Scorpio, et al., 1996; Scorpio, et al., 1997; Cheng, et al., 2000) and in view of the good correlation of pncA mutations and PZA resistance (Scorpio, et al., 1997; Cheng, et al., 2000; Somoskovi, et al., 2007), detection of pncA mutations by rapid molecular tests such as DNA sequencing is considered a good correlate of PZA resistance and can circumvent the problem of phenotype based PZA DST (Zhang, et al., 2012). However, a small percentage of PZA-resistant strains do not have pncA or rpsA mutations but due to other unknown resistance mechanisms (Zhang, et al., Microbiol Spectrum, 2014), therefore, molecular test detecting pncA mutations cannot replace phenotypic PZA susceptibility testing.

A SYBR Green I/PI viability assay for rapid assessment of Borrelia burgdorferi viability was recently developed (Feng, et al., 2014) and this method has successfully been used for high throughput drug screens against B. burgdorferi persisters (Feng, et al., Emerg Microbes Infect, 2014; Feng, et al., 2016). The presently disclosed subject matter provides the successful adaptation of this SYBR Green I/PI viability assay for rapid PZA DST in M. tuberculosis in 16 hrs.

### Methods

Isolation of Spontaneous PZA-Resistant Mutants of M. tuberculosis

Mycobacterium tuberculosis H37Ra was grown in 7H9 liquid medium (Difco) supplemented with 0.05% Tween 80 and 10% bovine serum albumin-dextrose-catalase (ADC) enrichment at 37° C. for approximately 10-14 days (mid- to late-log phase). Pyrazinamide (Sigma-Aldrich Co.) was dissolved in deionized water at a stock concentration of 10 mg/mL and filter-sterilized and incorporated into 7H11 agar plates containing ADC at concentrations of 100, 200, 300 μg/mL, pH6.0. Mutants that grew on the PZA containing plates after 3-4 week incubation at 37° C. were picked and grown in 7H9 liquid medium for confirming PZA resistance phenotype by repeated PZA susceptibility testing. The PZA susceptibility testing of the PZA-resistant mutants was performed on 7H11 agar plates containing 100, 200, 300 μg/mL PZA (pH6.0) as described (Zhang, et al., 2013).

### Microscopy Techniques

Specimens of M. tuberculosis cultures or cells were examined on a Zeiss Axiolmager M2 microscope equipped with epifluorescence illumination. Pictures were taken using ORCA-R² high resolution digital camera (HAMAMATSU, Japan). A SYBR Green I/PI assay was performed as previously described to assess cell viability using the ratio of green:red fluorescence to determine the live: dead cell ratio (Feng, et al., 2014). This residual cell viability was confirmed by analyzing three representative images of the bacterial culture using epifluorescence microscopy. Image Pro-Plus software was used to quantitatively determine the fluorescence intensity.

Establishing a Rapid Viability Test of M. tuberculosis Using SYBR Green I/PI Assay.

For assaying the live and dead cells in 96-well plates, SYBR Green I and PI were used for double staining of nucleic acids. SYBR Green I (10,000× stock, Invitrogen) (10 μL) was mixed with 30 μL propidium iodide (20 mM, Sigma) into 1.0 mL of sterile dH₂O and vortexed thoroughly. The staining mixture (10 μL) was added to each well (100 μL) and mixed thoroughly. The plate was incubated at room temperature in the dark for 45 minutes. With excitation wavelength at 485 nm, the fluorescence intensities at 538 nm (green emission) and 612 nm (red emission) were measured for each well of the plate using Synergy H1 microplate reader (BioTek Inc., USA).

Meanwhile the M. tuberculosis suspensions (live and 70% isopropyl alcohol killed) in five different proportions of live:dead cells (0:10, 2:8, 5:5, 8:2, 10:0) was mixed in wells of 96-well plate. The SYBR Green I/PI was added to each well and the green/red fluorescence ratios were measured for each proportion of live/dead M. tuberculosis using Synergy H1 microplate reader. With least-square fitting analysis, the regression equation and regression curve of the relationship between percentage of live bacteria and green/red fluorescence ratios were obtained. The regression equation was used to calculate the percentage of live cells in each well.

### SYBR Green I/PI Assay for Rapidly Testing PZA Susceptibility

The culture of wild type H37Ra (H37Ra WT) and PZA (Pyrazinamide) resistant mutants were respectively added into a 96-well plate, each well contains 100 μL culture (20 day old, 5×10⁶). 4 μL PZA (50 mg/mL) alone or with enhancer (salicylic acid 40 μg/mL, or acetic acid 8 μM) was added into 100 μL culture as PZA treated group; meanwhile untreated group was set as control. After one night incubation, 10 μL SYBR green I/PI staining mixture was added to 100 μL PZA treated and untreated cultures. The plate was incubated at room temperature in the dark for 45 minutes. The fluorescence intensities at 538 nm (green emission) and 612 nm (red emission) were measured for each well of the plate using Synergy H1 microplate reader (BioTek Inc., USA). Relative to the untreated group, the viability of PZA treated H37Ra WT and PZA resistant mutants were calculated according to the green and red fluorescence ratio. The difference in residual viability between H37Ra WT and PZA resistant mutants was compared by standard t-test.

### Results and Discussion

Isolation of Spontaneous PZA-Resistant Mutants of M. tuberculosis H37Ra

The parent M. tuberculosis strain H37Ra was susceptible to 100 μg/mL PZA (pH5.9). To isolate PZA-resistant spontaneous mutants, early stationary phase cultures of M. tuberculosis H37Ra were plated on 7H11 agar plates containing 200 μg/mL PZA (pH5.9). Four PZA resistant mutants were selected for developing the rapid PZA DST method by SYBR Green/PI assay as below.

A Rapid Viability Test for M. tuberculosis by SYBR Green I/PI Assay

SYBR Green I dye is commonly used for staining nucleic acids in gels and RT-PCR in molecular biology techniques. In previous studies with Borrelia burgdorferi, it was found that SYBR Green I/PI assay showed more sensitivity, more reliability and low background, than other viability assays such as MTT and XTT assays, FDA assay, commercial LIVE/DEAD BacLight kit, and the Sytox Green/Hoechst 33342 assay (Feng, et al., 2014). In the presently disclosed subject matter, the SYBR Green I/PI assay was adapted to be used as a rapid viability assay for M. tuberculosis.

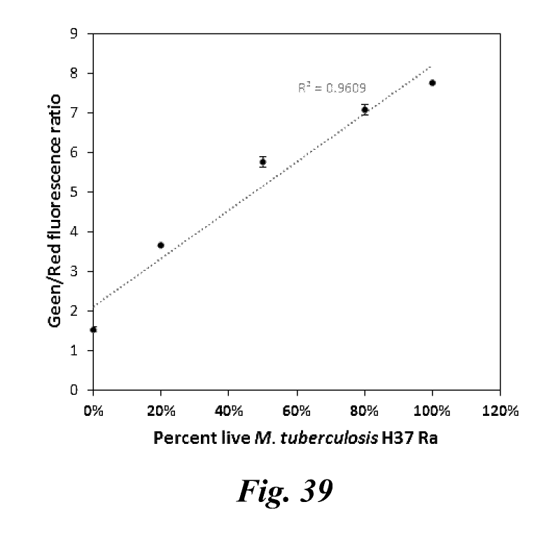

For M. tuberculosis H37Ra, the SYBR Green I/PI viability assay was optimized with a range of concentrations of SYBR Green I and PI dyes and tested with serial dilutions of M. tuberculosis culture. A linear relationship between the fluorescence ratio and the number of M. tuberculosis cells was found when the concentration of bacteria was higher than 1×10⁶ bacteria/mL. To validate the SYBR Green I/PI viability assay for M. tuberculosis, a mixture of live and dead M. tuberculosis cells in known proportions was used as standards. Live and 70% isopropyl alcohol killed M. tuberculosis cells were prepared in five different proportions (10⁸ bacteria/mL) in wells of the 96-well plate for the SYBR Green I/PI assay. The ratios of the integrated intensity of the portion of each spectrum at 538 nm (green) and 612 nm (red) for each bacterial suspension were calculated. The results are consistent with the results of fluorescence microscope counting, showing that the percentages of live bacteria correlated well with the ratio of green fluorescence to red fluorescence in a linear relationship for the SYBR Green/PI assay (FIG. 39).

### SYBR Green I/PI Assay for Rapid PZA Susceptibility Testing

The major impetus for this study is because there is no reliable PZA drug susceptibility testing (DST) method available. Thus there is a need to develop such a rapid PZA DST test for identifying PZA resistance for more effective clinical care. Since the SYBR Green I/PI assay was successfully adapted for rapid viability testing of M. tuberculosis (FIG. 1), whether the newly adapted SYBR Green I/PI assay could be used as a rapid PZA DST for detecting PZA resistance was evaluated.

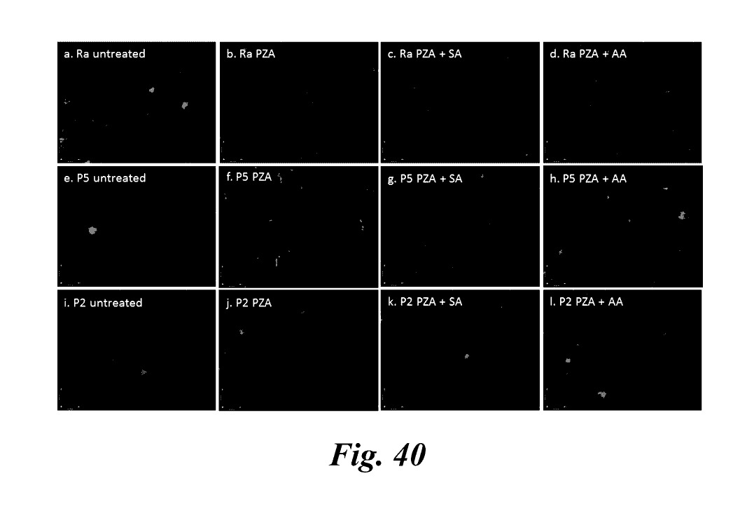

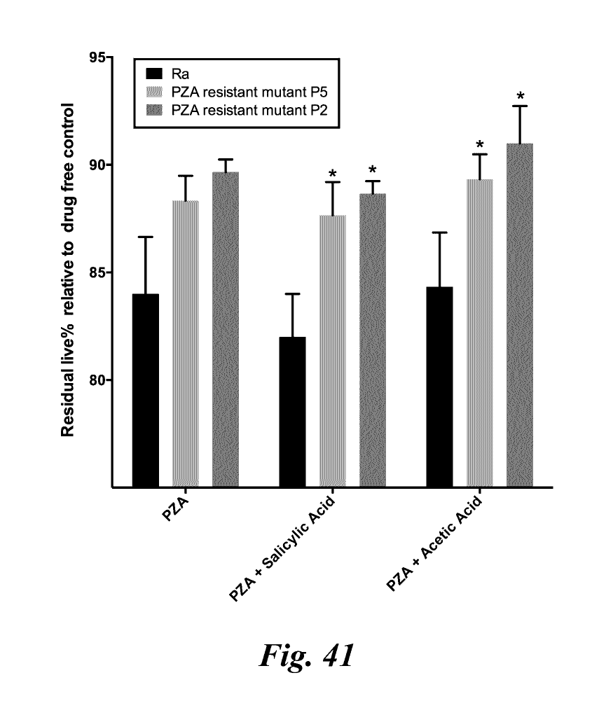

To do so, the residual viability of H37Ra wild type (WT) and the PZA resistant H37Ra mutants were compared after PZA treatment. The H37Ra WT and PZA resistant mutants were treated with different PZA concentrations (0.1, 0.5, 1 and 2 mg/mL) in 96-well microtiter plate, the SYBR Green I/PI assay was then used to determine the residual viability of the mycobacteria (see Methods). It was found that the residual viability difference between H37Ra WT and PZA resistant strains increased with higher PZA concentration in overnight (16 hr) treatment. Higher PZA concentration (2 mg/mL PZA) was more effective to kill M. tuberculosis H37Ra WT in a short time span while rapidly distinguishing PZA resistant mutants from drug susceptible H37Ra WT. After optimization, the residual viability of H37Ra WT was found to be obviously decreased as compared with the PZA resistant mutants after 2 mg/mL PZA overnight treatment (FIG. 40 and FIG. 41). This result provided the possibility to rapidly identify PZA resistance in less than one day.

Previous studies showed that PZA acts differently from common antibiotics by killing nonreplicating M. tuberculosis persisters (Zhang, et al., Microbiol Spectrum, 2014). Some weak acids and acid pH could enhance the activity of PZA against M. tuberculosis in vitro (Wade, et al., 2006; Zhang, et al., 1999). Therefore adding salicylic acid (SA) and acetic acid (AA) was tested in the PZA treatment to see if they help to show more significant difference in residual viability between parent drug susceptible strain H37Ra and PZA resistant mutants. It was found that the 40 μg/mL salicylic acid or 8 μM acetic acid could indeed enhance the activity of PZA against H37Ra WT but not PZA resistant mutants at 2 mg/mL PZA in overnight treatment (FIG. 40 and FIG. 41). Using the SYBR Green I/PI assay, the residual viability of H37Ra WT was significantly lower than the residual viability of the two PZA resistant mutants P5, P2 (FIG. 41), and their difference achieved statistical significance (P<0.05). These results showed that the SYBR Green I/PI assay with weak acids could quantitatively assay the PZA resistance in M. tuberculosis in less than 1 day. This provides proof of principle test for performing DST with the newly developed SYBR Green I/PI assay for PZA and all other TB drugs in less than one day, thus revolutionizing TB drug susceptibility testing, which currently takes weeks to complete and often with expensive instrumentation.

Previous studies have shown that flow cytometry can be used for PZA DST for M. tuberculosis in 24 hr (Fredericks, et al., 2006). However, this method still relies on detecting of growth by fluoroscein diacetate, and the most significant drawbacks are the requirement for expensive instrumentation of flow cytometer and lack of high throughput. Because of these shortcomings, the flow cytometry method has not been adopted by clinical or field settings.

In summary, the SYBR Green I/PI assay was adapted for viability assessment of M. tuberculosis and developed a rapid PZA DST, reducing the time required for DST results from previously weeks to less than 1 day. This is made possible using the SYBR Green I/PI viability assay by converting a conventional growth/culture-dependent DST to a viability based and growth/culture-independent DST. The presently disclosed subject matter is the first to demonstrate the feasibility and reliability of the adapted SYBR Green I/PI assay for rapidly identifying PZA resistant M. tuberculosis mutants. The SYBR Green I/PI assay overcomes the inefficiency of classic microbiology growth/culture based DST relying on growth of slow growing M. tuberculosis (which takes several weeks) and the limitations of molecular testing methods (with compromised sensitivity compared with phenotype based DST as mutations are not all covered by molecular tests). The SYBR Green I/PI assay can be developed as a convenient and accurate test for rapid TB DST for all TB drugs and thus provide timely feedback for guiding clinical treatment.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Alban, P. S., et al., Serum-starvation-induced changes in protein synthesis and morphology of Borrelia burgdorferi. Microbiology 2000, 146 (Pt 1): 119-127.
Amant, F., et al., (2012) Chemotherapy during pregnancy. Curr. Opin. Oncol. 2012, 24: 580-586.
Ando H, Mitarai S, Kondo Y, Suetake T, Sekiguchi J I, et al. (2010) Pyrazinamide resistance in multidrug-resistant Mycobacterium tuberculosis isolates in Japan. Clin Microbiol Infect 16: 1164-1168.
Aragon L M, Garrigo M, Moreno C, Espanol M, Coll P (2007) Evaluation of the BacT/ALERT PZA kit in comparison with the BACTEC 460 TB PZA for testing Mycobacterium tuberculosis susceptibility to pyrazinamide. J Antimicrob Chemother 60: 655-657.
Arbiser, J. L. and Moschella, S. L., Clofazimine: a review of its medical uses and mechanisms of action. J. Am. Acad Dermatol. 1995, 32(2 Pt 1): p. 241-7.
Armed Forces Health Surveillance, Center, Surveillance snapshot: Lyme disease among beneficiaries of the Military Health System, 2001-2012. MSMR 2013, 20(8): p. 23.
Barbesti, S., et al., Two and three-color fluorescence flow cytometric analysis of immunoidentified viable bacteria. Cytometry 2000, 40:214-8.
Bacon, R. M., et al., Surveillance for Lyme disease—United States, 1992-2006. MMWR Surveill. Summ. 2008, 57(10): p. 1-9.
Barthold, S. W., et al., Ineffectiveness of tigecycline against persistent Borrelia burgdorferi. Antimicrobial Agents and Chemotherapy 2010, 54: 643-651.
Bergström, S. and Zückert, W. R., Structure, function and biogenesis of the Borrelia cell envelope, in Borrelia: Molecular Biology, Host Interaction and Pathogenesis. Caister Academic Press 2010, p. 139-166.
Berndtson, K., Review of evidence for immune evasion and persistent infection in Lyme disease. Int. J. Gen. Med. 2013, 6:291-306.
Bockenstedt, L. K., et al., Spirochete antigens persist near cartilage after murine Lyme borreliosis therapy. J. Clin. Invest. 2012, 122: 2652-2660.
Boemer, J., et al., In vitro antimicrobial susceptibility testing of Borrelia burgdorferi: influence of test conditions on minimal inhibitory concentration (MIC) values. Zentralbl Bakteriol 1995, 283(1): p. 49-60.
Boulos, L., et al., LIVE/DEAD BacLight: application of a new rapid staining method for direct enumeration of viable and total bacteria in drinking water. J. Microbiol. Methods 1999, 37:77-86.
Brorson, O., and Brorson, S. H. Transformation of cystic forms of Borrelia burgdorferi to normal, mobile spirochetes. Infection 1997, 25: 240-246.
Brorson, O. and Brorson, S. H. In vitro conversion of Borrelia burgdorferi to cystic forms in spinal fluid, and transformation to mobile spirochetes by incubation in BSK-H medium. Infection 1998, 26: 144-150.
Brorson, O. and Brorson, S. H. An in vitro study of the susceptibility of mobile and cystic forms of Borrelia burgdorferi to metronidazole. APMIS 1999, 107(6): p. 566-76.
Brorson, O., et al., Destruction of spirochete Borrelia burgdorferi round-body propagules (RBs) by the antibiotic tigecycline. PNAS 2009, 106(44): p. 18656-61.
Brouqui, P., et al., Eucaryotic cells protect Borrelia burgdorferi from the action of penicillin and ceftriaxone but not from the action of doxycycline and erythromycin. Antimicrob. Agents Chemother. 1996, 40(6): p. 1552-1554.
Campbell, J. W. and Cronan, J. E., Jr., Bacterial fatty acid biosynthesis: targets for antibacterial drug discovery. Annu. Rev. Microbiol. 2001; 55:305-32.
Casjens, S. (2000) Borrelia genomes in the year 2000. J. Mol. Microbiol. Biotechnol. 2000, 2: 401-410.
Centers for Disease Control (2012 [cited by 2014 February]) Lyme disease data.
Centers for Disease Control (2014) Post-Treatment Lyme Disease Syndrome.
Chang K C, Leung C C, Yew W W, Lau T Y, Tam C M (2008) Hepatotoxicity of pyrazinamide: cohort and case-control analyses. American Journal of Respiratory and Critical Care Medicine 177: 1391-1396.
Chedore P, Bertucci L, Wolfe J, Sharma M, Jamieson F (2010) Potential for erroneous results indicating resistance when using the Bactec MGIT 960 system for testing susceptibility of Mycobacterium tuberculosis to pyrazinamide. Journal of Clinical Microbiology 48: 300-301.
Cheng S J, Thibert L, Sanchez T, Heifets L, Zhang Y (2000) pncA mutations as a major mechanism of pyrazinamide resistance in Mycobacterium tuberculosis: spread of a monoresistant strain in Quebec, Canada. Antimicrob Agents Chemother 44: 528-532.
Cholo, M. C., et al., Clofazimine: current status and future prospects. J. Antimicrob. Chemother. 2012, 67: 290-298.
Chong, C. R., et al., A clinical drug library screen identifies astemizole as an antimalarial agent. Nat. Chem. Biol. 2006, 2(8): p. 415-6.
Clinical and Laboratory Standards Institute, Performance Standards for Antimicrobial Susceptibility Testing; Seventeenth Informational Supplement. CLSI document M100-S17 2007, 27(1): p. 154-161.
CLSI (2011) Susceptibility Testing of Mycobacteria, Nocardiae, and Other Aerobic Actinomycetes; Approved Standard-Second Edition. CLSI document M24-A2. Wayne, Pa.: Clinical and Laboratory Standards Institute.
CLSI (2016) Performance Standards for Antimicrobial Performance Standards for Antimicrobial Susceptibility Testing. 26th ed. CLSI supplement M100S. Wayne, Pa.: Clinical and Laboratory Standards Institute.
Dever, L. L., et al., In vitro antimicrobial susceptibility testing of Borrelia burgdorferi: a microdilution MIC method and time-kill studies. J. Clin. Microbiol. 1992, 30(10): p. 2692-7.
Dever, L. L., et al., In vitro activity of vancomycin against the spirochete Borrelia burgdorferi. Antimicrob Agents Chemother. 1993, 37: 1115-1121.
Dhand, A., et al., Use of antistaphylococcal beta-lactams to increase daptomycin activity in eradicating persistent bacteremia due to methicillin-resistant Staphylococcus aureus: role of enhanced daptomycin binding. Clin. Infect. Dis. 2011, 53: 158-163.
Diterich, I., et al., Borrelia burgdorferi-induced tolerance as a model of persistence via immunosuppression. Infect. Immun. 2003, 71(7): p. 3979-87.
DTP—Mechanistic Set Information. 2015 [cited 2015].
DTP—Natural Products Set information. 2015 [cited 2015].
Embers, M. E., et al. Persistence of Borrelia burgdorferi in Rhesus Macaques following Antibiotic Treatment of Disseminated Infection. PLoS One 2012, 7(1):e29914.
Fallon, B. A., et al., A randomized, placebo-controlled trial of repeated IV antibiotic therapy for Lyme encephalopathy. Neurology 2008, 70: 992-1003.
Feng J, Shi W, Zhang S, Zhang Y (2015) Identification of new compounds with high activity against stationary phase Borrelia burgdorferi from the NCI compound collection. Emerg Microbes Infect 4: e31.
Feng J, Shi W, Zhang S, Sullivan D, Auwaerter P G, et al. (2016) A Drug Combination Screen Identifies Drugs Active against Amoxicillin-Induced Round Bodies of In Vitro Borrelia burgdorferi Persisters from an FDA Drug Library. Front Microbiol 7: 743.
Feng, J., Wang, T., Shi, W., et al., Identification of novel activity against Borrelia Burgdorferi persisters using an FDA approved drug library. Emerging Microbes and Infections 2014, e49.
Feng, J., Wang, T., Zhang, S., et al., An Optimized SYBR Green I/PI Assay for Rapid Viability Assessment and Antibiotic Susceptibility Testing for Borrelia burgdorferi. PLoS One 2014, 9: e111809.
Fredricks B A, DeCoster D J, Kim Y, Sparks N, Callister S M, et al. (2006) Rapid pyrazinamide susceptibility testing of Mycobacterium tuberculosis by flow cytometry. J Microbiol Methods 67: 266-272.
Gabrielson, J., et al., Evaluation of redox indicators and the use of digital scanners and spectrophotometer for quantification of microbial growth in microplates. J Microbiol Methods 2002, 50:63-73.
Gregori, G., et al., Resolution of viable and membrane-compromised bacteria in freshwater and marine waters based on analytical flow cytometry and nucleic acid double staining. Appl. Environ. Microbiol. 2001, 67:4662-70.
Hall Snyder, A., et al., Evaluation of the novel combination of daptomycin plus ceftriaxone against vancomycin-resistant enterococci in an in vitro pharmacokinetic/pharmacodynamic simulated endocardial vegetation model. The Journal of Antimicrobial Chemotherapy 2014, 69: 2148-2154.
Hayashi, M., et al., The mode of action of nanaomycins D and A on a gram-negative marine bacterium Vibrio alginolyticus. J. of Antibiotics. 1982; 35(8):1078-85.
Hewlett D, Jr., Horn D L, Alfalla C (1995) Drug-resistant tuberculosis: inconsistent results of pyrazinamide susceptibility testing. JAMA 273: 916-917.
Hodzic, E., et al., Resurgence of Persisting Non-Cultivable Borrelia burgdorferi following Antibiotic Treatment in Mice PLoS One. 2014, 9: e86907.
Hodzic, E., et al., Persistence of Borrelia burgdorferi following antibiotic treatment in mice. Antimicrobial Agents and Chemotherapy 2008, 52:1728-36.
Hollstein, U. Actinomycin. Chemistry and mechanism of action. Chemical Reviews. 1974; 74(6):625-52.
Hughes A J, Hutchinson P, Gooding T, Freezer N J, Holdsworth S R, et al. (2005) Diagnosis of Mycobacterium tuberculosis infection using ESAT-6 and intracellular cytokine cytometry. Clin Exp Immunol 142: 132-139.
Hunfeld, K. P. and Brade, V., Antimicrobial susceptibility of Borrelia burgdorferi sensu lato: what we know, what we don't know, and what we need to know. Wien Klin Wochenschr. 2006, 118(21-22): p. 659-68.
Jacobs W R, Jr., Barletta R G, Udani R, Chan J, Kalkut G, et al. (1993) Rapid assessment of drug susceptibilities of Mycobacterium tuberculosis by means of luciferase reporter phages. Science 260: 819-822.
Jensen, P. B., et al., Different modes of anthracycline interaction with topoisomerase II. Separate structures critical for DNA-cleavage, and for overcoming topoisomerase II-related drug resistance. Biochem. Pharmacol. 1993; 45(10):2025-35.
Jonmalung J, Prammananan T, Leechawengwongs M, Chaiprasert A (2010) Surveillance of pyrazinamide susceptibility among multidrug-resistant Mycobacterium tuberculosis isolates from Siriraj Hospital, Thailand. BMC Microbiol 10: 223.
Keren, I., et al., Persister cells and tolerance to antimicrobials. FEMS Microbiol. Lett. 2004, 230:13-18.
Kersten, A., et al., Effects of penicillin, ceftriaxone, and doxycycline on morphology of Borrelia burgdorferi. Antimicrob. Agents Chemother. 1995, 39(5): p. 1127-33.
Klempner, M. S. et al., Treatment trials for post-Lyme disease symptoms revisited. Am. J. Med. 2013, 126: 665-669.
Kraiczy, P., et al., In vitro activities of fluoroquinolones against the spirochete Borrelia burgdorferi. Antimicrob. Agents Chemother. 2001, 45: 2486-2494.
Krupp, L. B., et al., Study and treatment of post Lyme disease (STOP-LD): a randomized double masked clinical trial. Neurology 2003, 60: 1923-1930.
Lantos, P. M., et al. A systematic review of Borrelia burgdorferi morphologic variants does not support a role in chronic Lyme disease. Clin. Infect. Dis. 2014, 58: 663-671.
Li, W., et al., Yeast model uncovers dual roles of mitochondria in action of artemisinin. PLoS Genet 2005, 1: e36.
Li, Y. and Zhang, Y. PhoU is a persistence switch involved in persister formation and tolerance to multiple antibiotics and stresses in Escherichia coli. Antimicrob. Agents Chemother. 2007, 51: 2092-2099.
Louw G E, Warren R M, Donald P R, Murray M B, Bosman M, et al. (2006) Frequency and implications of pyrazinamide resistance in managing previously treated tuberculosis patients. Int J Tuberc Lung Dis 10: 802-807.
Lu, S., et al., Bis-naphtho-gamma-pyrones from fungi and their bioactivities. Molecules. 2014; 19(6):7169-88.
Marques, A., et al., Xenodiagnosis to detect Borrelia burgdorferi Infection: A first-in-Human Study. Clinical Infectious Diseases 2014, 58 (7): 937-945.
Meek, J. I., et al., Underreporting of Lyme disease by Connecticut physicians, 1992. J. Public Health. Pract. 1996, 2(4): p. 61-65.
Migliori G B, Besozzi G, Girardi E, Kliiman K, Lange C, et al. (2007) Clinical and operational value of the extensively drug-resistant tuberculosis definition. European Respiratory Journal 30: 623-626.
Miklossy, J., et al., Persisting atypical and cystic forms of Borrelia burgdorferi and local inflammation in Lyme neuroborreliosis. J. of Neuroinflammation 2008, 5:40.
Mitchison D A (1985) The action of antituberculosis drugs in short course chemotherapy. Tubercle 66: 219-225.
Mizuno, N. S., et al., Binding of daunomycin to DNA and the inhibition of RNA and DNA synthesis. Cancer Res. 1975; 35(6):1542-6.
Mole R, Trollip A, Abrahams C, Bosman M, Albert H (2007) Improved contamination control for a rapid phage-based rifampicin resistance test for Mycobacterium tuberculosis. J Med Microbiol 56: 1334-1339.
Moody, M. R., et al., Effect of two cancer chemotherapeutic agents on the antibacterial activity of three antimicrobial agents. Antimicrob. Agents Chemother. 1978; 14(5):737-42.
Morris, C. M., et al., Effect of polymyxin B nonapeptide on daptomycin permeability and cell surface properties in Pseudomonas aeruginosa, Escherichia coli, and Pasteurella multocida. J Antibiot. (Tokyo) 1995, 48: 67-72.
Mshana, R. N., et al., Use of 3-(4,5-dimethylthiazol-2-yl)-2,5-diphenyl tetrazolium bromide for rapid detection of rifampin-resistant Mycobacterium tuberculosis. J Clin. Microbiol. 1998, 36:1214-9.
Murgia, R. and Cinco, M. Induction of cystic forms by different stress conditions in Borrelia burgdorferi. APMIS: acta pathologica, microbiologica, et immunologica Scandinavica 2004, 112: 57-62.
Niu H, Peng Cui, Rebecca Yee, Wanliang Shi, Shuo Zhang, Jie Feng, David Sullivan, Wenhong Zhang, Bingdong Zhu, Ying Zhang (2015) A Clinical Drug Library Screen Identifies Tosufloxacin as Being Highly Active against Staphylococcus aureus Persisters. Antibiotics 4: 329-336.
Niu H, Cui P, Shi W, Zhang S, Feng J, et al. (2015) Identification of Anti-Persister Activity against Uropathogenic Escherichia coli from a Clinical Drug Library. Antibiotics (Basel) 4: 179-187.
Open Repository Collection of Synthetics and Pure Natural Products. 2014
[cited 2015 Mar. 15].
Orloski, K. A., et al., Surveillance for Lyme disease—United States, 1992-1998. MMWR CDC Surveill Summ. 2000, 49(3): p. 1-11.
Palaniappan, K., and Holley, R. A. Use of natural antimicrobials to increase antibiotic susceptibility of drug resistant bacteria. International Journal of Food Microbiology 2010, 140: 164-168.
Pogliano, J., et al., Daptomycin-mediated reorganization of membrane architecture causes mislocalization of essential cell division proteins. J Bacteriol. 2012, 194(17): p. 4494-504.
Pommier, Y., et al., DNA topoisomerases and their poisoning by anticancer and antibacterial drugs. Chemistry & Biology. 2010; 17(5):421-33.
Radolf, J. D., et al., Of ticks, mice and men: understanding the dual-host lifestyle of Lyme disease spirochaetes. Nat. Rev. Microbiol. 2012, 10: 87-99.
Rao N A, Irfan M, Soomro M M, Mehfooz Z (2010) Drug resistance pattern in multidrug resistance pulmonary tuberculosis patients. J Coll Physicians Surg Pak 20: 262-265.
Ricker, L. J., et al. Persistent subfoveal fluid and increased preoperative foveal thickness impair visual outcome after macula-off retinal detachment repair. Retina 2011, 31: 1505-1512.
Sapi, E., et al., Characterization of biofilm formation by Borrelia burgdorferi in vitro. Plos One 2012, 7(10): p. e48277.
Sapi, E., et al., Evaluation of in-vitro antibiotic susceptibility of different morphological forms of Borrelia burgdorferi. Infect. Drug Resist. 2011, 4: p. 97-113.
Scorpio A, Lindholm-Levy P, Heifets L, Gilman R, Siddiqi S, et al. (1997) Characterization of pncA mutations in pyrazinamide-resistant Mycobacterium tuberculosis. Antimicrob Agents Chemother 41: 540-543.
Scorpio A, Zhang Y (1996) Mutations in pncA, a gene encoding pyrazinamidase/nicotinamidase, cause resistance to the antituberculous drug pyrazinamide in tubercle bacillus. Nat Med 2: 662-667.
Senol G, Coskun M, Gunduz A T, Bicmen C, Gayaf M, et al. (2008) [Investigation of pyrazinamide resistance in multidrug-resistant tuberculosis cases in Hospital of Pulmonary Diseases, Izmir, Turkey]. Mikrobiyoloji Bulteni 42: 591-597.
Shenai S, Rodrigues C, Sadani M, Sukhadia N, Mehta A (2009) Comparison of phenotypic and genotypic methods for pyrazinamide susceptibility testing. Indian J Tuberc 56: 82-90.
Shopov, A., et al., Improvements in image analysis and fluorescence microscopy to discriminate and enumerate bacteria and viruses in aquatic samples. Aquatic Microbial Ecology 2000, 22:103-110.
Simpson G, Coulter C, Weston J, Knight T, Carter R, et al. (2011) Resistance patterns of multidrug-resistant tuberculosis in Western Province, Papua New Guinea. Int J Tuberc Lung Dis 15: 551-552.
Sobell, H. M. Actinomycin and DNA transcription. Proceedings of the National Academy of Sciences. 1985; 82(16):5328-31.
Somoskovi A, Dormandy J, Parsons L M, Kaswa M, Goh K S, et al. (2007) Sequencing of the pncA gene in members of the Mycobacterium tuberculosis complex has important diagnostic applications: Identification of a species-specific pncA mutation in “Mycobacterium canettii” and the reliable and rapid predictor of pyrazinamide resistance. J Clin Microbiol 45: 595-599.
Steere, A. C. and Glickstein, L., Elucidation of Lyme arthritis. Nat. Rev. Immunol. 2004, 4: 143-152.
Straubinger, R. K., et al., Persistence of Borrelia burgdorferi in experimentally infected dogs after antibiotic treatment. Journal of Clinical Microbiology 1997, 35: 111-116.
Stricker, R. B. and L. Johnson, Lyme disease: the next decade. Infect. Drug Resist. 2011, 4: p. 1-9.
Sun, Z. and Zhang, Y., Spent culture supernatant of Mycobacterium tuberculosis H37Ra improves viability of aged cultures of this strain and allows small inocula to initiate growth. Journal of Bacteriology 1999, 181:7626-7628.
Szybalski, W. and Iyer, V. N. Crosslinking of DNA by Enzymatically or Chemically Activated Mitomycins and Porfiromycins, Bifunctionally “Alkylating” Antibiotics. Federation Proceedings. 1964; 23:946-57.
Tan, C., et al., Daunomycin, an antitumor antibiotic, in the treatment of neoplastic disease. Clinical evaluation with special reference to childhood leukemia. Cancer 1967; 20(3):333-53.
Taylor, J. P., et al., Comparison of microbial numbers and enzymatic activities in surface soils and subsoils using various techniques. Soil Biology & Biochemistry 2002, 34:387-401.
Tomasz, M., Mitomycin C: small, fast and deadly (but very selective). Chem. Biol. 1995; 2(9):575-9.
Vanden Bossche, H., et al., Biochemical approaches to selective antifungal activity. Focus on azole antifungals. Mycoses 1989, 32 Suppl 1: 35-52.
Van Rensburg, C. E., et al., Antimicrobial activities of clofazimine and B669 are mediated by lysophospholipids. Antimicrob. Agents Chemother. 1992, 36: 2729-2735.
Velayati A A, Masjedi M R, Farnia P, Tabarsi P, Ghanavi J, et al. (2009) Emergence of new forms of totally drug-resistant tuberculosis bacilli: super extensively drug-resistant tuberculosis or totally drug-resistant strains in iran. Chest 136: 420-425.
Wade M M, Zhang Y (2006) Effects of weak acids, U V and proton motive force inhibitors on pyrazinamide activity against Mycobacterium tuberculosis in vitro. J Antimicrob Chemother 58: 936-941.
Wang, H., Cheng, H., et al., An improved 3-(4,5-dimethylthiazol-2-yl)-2,5-diphenyl tetrazolium bromide (MTT) reduction assay for evaluating the viability of Escherichia coli cells. J Microbiol Methods 2010, 82:330-3.
Wang, J., Huang, L., et al. Artemisinin directly targets malarial mitochondria through its specific mitochondrial activation. Plos One 2010, 5: e9582.
Welch, H., The antibiotic saga. The University of Michigan 1960, p. 96.
Wells, T. N., et al., New medicines to improve control and contribute to the eradication of malaria. Nat. Rev. Drug Discov. 2009, 8: 879-891.
WHO (2008) Anti-tuberculosis Drug Resistance in the World, Report No. 4.
Wikler, M. A., and Ferraro, M. J., Correction of a reference to clinical laboratory standards institute interpretive criteria. Clin. Infect. Dis. 2008, 46: 1798; author reply 1798-1799.
Williamson, N. R., et al., Anticancer and immunosuppressive properties of bacterial prodiginines. Future Microbiol. 2007; 2(6):605-18.
Wilson S M, al-Suwaidi Z, McNerney R, Porter J, Drobniewski F (1997) Evaluation of a new rapid bacteriophage-based method for the drug susceptibility testing of Mycobacterium tuberculosis. Nat Med 3: 465-468.
Wormser, G. P., et al., The clinical assessment, treatment, and prevention of Lyme disease, human granulocytic anaplasmosis, and babesiosis: clinical practice guidelines by the Infectious Diseases Society of America. Clin. Infect. Dis. 2006, 43: 1089-1134.
Xu, J., et al., In vitro and in vivo activity of clofazimine against Mycobacterium tuberculosis persisters. Int. J. Tuberc. Lung Dis. 2012, 16: 1119-1125.
Zhang, Y., Persisters, persistent infections and the Yin-Yang Model. Emerging Microbes & Infections 2014, 3, e3.
Zhu, L., et al., Syntheses and biological activities of daunorubicin analogs with uncommon sugars. Bioorganic & Medicinal Chemistry. 2005; 13(23):6381-7.
Zhang S, Chen J, Shi W, Liu W, Zhang W H, et al. (2013) Mutations in panD encoding aspartate decarboxylase are associated with pyrazinamide resistance in Mycobacterium tuberculosis. Emerging Microbes & Infections 2: e34; doi: 10.1038/emi.2013.1038.
Zhang Y, Chang K, Leung C, Yew W, Gicquel G, et al. (2012) “ZS-MDR-TB” versus “ZR-MDR-TB”: Improving Treatment of MDR-TB by Identifying Pyrazinamide Susceptibility. Emerging Microbes and Infections (Nature Publishing Group) 1: e5; doi:10.1038/emi.2012.1018.
Zhang Y, Mitchison D (2003) The curious characteristics of pyrazinamide: a review. Int J Tuberc Lung Dis 7: 6-21.
Zhang Y, Scorpio A, Nikaido H, Sun Z (1999) Role of acid pH and deficient efflux of pyrazinoic acid in unique susceptibility of Mycobacterium tuberculosis to pyrazinamide. J Bacteriol 181: 2044-2049.
Zhang Y, Shi W, Zhang W, Mitchison D (2014) Mechanisms of Pyrazinamide Action and Resistance. Microbiol Spectrum, ASM Press 2(4): July 2014, 2012:2012.2014.2003. doi:2010.1128/microbiolspec.MGM2012-0023-2013.

Although the foregoing subject matter has been described in some detail by way of illustration and example for purposes of clarity of understanding, it will be understood by those skilled in the art that certain changes and modifications can be practiced to include other microbes within the scope of the appended claims.

That which is claimed:

1. A method for assessing the viability or antimicrobial susceptibility of bacteria from a selected genus, the method comprising:
(a) establishing a bacterial culture comprising isolated bacteria from the selected genus;
(b) incubating the bacterial culture with a staining mixture comprising:
(i) a first agent which emits fluorescence of a first color that is indicative of live bacterial cells in the culture, and
(ii) a second agent which emits fluorescence of a second color that contrasts from the first color and is indicative of dead bacterial cells in the culture; and
(c) calculating a ratio of the intensity of emitted fluorescence of the first color to the intensity of emitted fluorescence of the second color; and
(d) assessing the viability of the bacteria in the culture, wherein the ratio calculated in (c) is indicative of the percentage of live bacteria in the culture.

2. The method of claim 1, wherein the first fluorescent agent is SYBR Green I and the second fluorescent agent is propidium iodide.

3. The method of claim 1, wherein the selected genus is selected from the group consisting of Borrelia, Staphylococcus, Escherichia, Klebsiella, Acinetobacter, and Mycobacterium.

4. The method of claim 1, further comprising assessing the susceptibility of bacteria from a selected genus to at least one antimicrobial agent by incubating the culture of step (a) under suitable conditions for bacterial growth to occur with at least one dose of at least one antimicrobial agent and assessing the susceptibility of bacteria from the selected genus to at least one antimicrobial agent by calculating the ratio in step (b) without the need for bacterial growth, after a period of exposure to the at least one dose of the at least one antimicrobial agent, wherein the bacteria are assessed as susceptible to the at least one antimicrobial agent if the ratio in step (b) remains the same or decreases after the period of exposure to the at least one dose of the at least one antimicrobial agent, and wherein the bacteria are assessed as resistant to the at least one antimicrobial agent if the ratio in step (b) increases after the period of exposure to the at least one dose of the at least one agent.

5. The method of claim 4, further comprising determining a minimum inhibitory concentration breakpoint for the at least one antimicrobial agent.

6. The method of claim 1, further comprising identifying a candidate agent that is capable of inhibiting growth or survival of bacteria from the selected genus by contacting the culture of step (a) with a test agent and assessing a viability of the bacteria in the culture in the presence of the test agent as compared to the viability of the bacteria in a control culture which lacks the test agent.

7. The method of claim 6, wherein the culture comprises a stationary phase culture comprising non-replicating persister cells.

8. A kit for rapid screening or antimicrobial susceptibility testing of at least one candidate agent that is capable of inhibiting growth or survival of bacteria from a selected genus, the kit comprising:
(a) a population of isolated bacteria comprising bacteria from the Borellia genus or a culture thereof;
(b) a staining mixture comprising:
(i) a first agent which emits fluorescence of a first color that is indicative of live bacterial cells, and
(ii) a second agent which emits fluorescence of a second color that contrasts from the first color and is indicative of dead bacterial cells, wherein when the staining mixture is incubated with the bacteria population or culture thereof a calculated ratio of the intensity of emitted fluorescence of the first color to the intensity of emitted fluorescence the second color is indicative of the percentage of live bacteria in the population or culture thereof; and
(c) instructions for using the bacteria in (a) and the staining mixture in (b) to screen for at least one candidate agent that is capable of inhibiting growth or survival of bacteria from the selected genus.

9. The kit of claim 8, further comprising at least one test agent to screen or test for antimicrobial susceptibility for its ability to inhibit the growth or survival of bacteria from the selected genus, wherein the selected genus is selected from the group consisting of Borrelia, Staphylococcus, Escherichia, Klebsiella, Acinetobacter, and Mycobacterium.

10. The kit of claim 8, further comprising instructions for one or more of the following:
(i) contacting the population of bacteria or population thereof with the at least one test agent;
(ii) incubating the staining mixture with the population of bacteria or culture thereof;
(iii) assessing the viability of the bacteria in the population or culture thereof; and
(iv) instructions for calculating a ratio of the intensity of emitted fluorescence of the first color to the intensity of emitted fluorescence the second color.

11. The kit of claim 8, wherein the bacteria are selected from the group consisting of Borrelia burgdorferi, Staphylococcus aureus, Escherichia coli, Klebsiella pneumoniae, Acinetobacter baumanii, and Mycobacterium tuberculosis.

12. The kit of claim 8, wherein the culture comprises a stationary phase culture comprising non-replicating persister cells.

13. The kit of claim 8, wherein the first agent is SYBR Green I and the second agent is propidium iodide.

14. A method for treating a bacterial infection from a selected genus in a subject in need of treatment thereof, the method comprising administering to the subject an effective amount of:
(a) at least one compound selected from the group consisting of daptomycin, artemisinin, ciprofloxacin, sulfacetamide, sulfamethoxypyridazine, nifuroxime, fosfomycin, chlortetracycline, sulfathiazole, clofazimine, cefmenoxime, cefoperazone, carbomycin, cefotiam, cefepime, amodiaquin, fosfomycin and streptomycin;
(b) at least one compound selected from the group consisting of daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone;
(c) a combination of at least two compounds comprising:
(i) a first compound selected from the group consisting of daptomycin, cefoperazone, miconazole and sulfamethoxypyridazine; and
(ii) a second compound other than the first compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone; or
(d) a combination of at least three compounds comprising:
(i) doxycycline as a first compound;
(ii) a second compound selected from the group consisting of daptomycin or cefoperazone; and
(iii) a third compound other than the second compound selected from the group consisting of daptomycin, amoxicillin, cefuroxime, ceftriaxone, miconazole, doxycycline, carbenicillin, clofazimine, artemisinin, ciprofloxacin, sulfacetamide, sulfamethoxypyridazine, sulfachlorpyridazine, nifuroxime, nitrofurantoin, fosfomycin, chlortetracycline, sulfathiazole, clofazimine, chlortetracycline, cefmenoxime, cefmetazole, cefoperazone, carbomycin, cefotiam, cefepime, amodiaquin, fosfomycin, streptomycin, daunomycin 3-oxime; dimethyldaunomycin; daunorubicin; 9,10-anthracenedione, 1-hydroxy-4-[[2-[(2-hydroxyethyl)amino]ethyl]amino]-; anthracene-9,10-dione, 1,5-bis[3-[[(2-hydroxyethyl)amino]propyl]amino]-9,10-dihydro-; nogalamycin; pyronin B; N-allyl-2-(methylthio)[1,3]thiazolo[5,4-d]pyrimidin-7-amine; pyrromycin; rhodomycin A; chaetochromin; 9,10-anthracenedione, 1,4-dihydroxy-2-[[2-[(2-hydroxyethyl)amino]ethyl]amino]-; prodigiosin; mitomycin; nanaomycin; 9-hydroxy-2-(2-piperidinylethyl)ellipticinium acetate; N-[3-(2-pyridyl)isoquinolin-1-yl]-2-pyridinecarboxamidine; naphthalene-1,4-dione, 2-chloro-5,8-dihydroxy-3-(2-methoxyethoxy)-; 9H-thioxanthen-9-one, 1-[[2-(dimethylamino)ethyl]amino]-7-hydroxy-4-methyl-,monohydriodide; dactinomycin; emodin; (5,8-dihydroxy-1,4-dioxo-1,4-dihydronaphthalene-2,3-diyl)dimethanediyl dicarbamate; 1-phenazinecarboxamide, N-[2-(dimethylamino)ethyl]-6,9-dimethoxy-; (5-phenyl-1,3-thiazol-2-yl)methanol; 3,3′,4′,7-tetrahydroxyflavone; benzoic acid, 2-hydroxy-, (2,6-pyridinediyldiethylidyne) dihydrazide, nickel complex; 1-(1,2-dihydro-5-acenaphthylenyl)-N-hydroxy-1-phenylmethanimine; 2-methyl-4,4′-[(4-imino-2,5-cyclohexadien-1-ylidene)methylene]dianiline; 3,3′-diethyl-9-methylthiacarbocyanine iodide; and 1,8-di(phenylthio)anthraquinone.

15. The method of claim 14, wherein the bacteria are selected from the group consisting of Borrelia burgdorferi, Staphylococcus aureus, Escherichia coli, Klebsiella pneumoniae, Acinetobacter baumanii, and Mycobacterium tuberculosis.

16. The method of claim 14, wherein the bacteria comprise replicating forms of the bacteria, non-replicating persister forms of the bacteria, and combinations of replicating forms of the bacteria and non-replicating persister forms of the bacteria.

17. The method of claim 14, wherein the disease or condition is Lyme disease.

18. The method of claim 17, wherein the subject has, or is suspected of having, post-treatment Lyme disease syndrome (PTLDS) and/or antibiotic refractory Lyme arthritis.

19. The method of claim 14, further comprising:
(a) administering to the subject an effective amount of a combination of at least two agents comprising:
(i) at least one agent that inhibits growth and/or survival of replicating forms of bacteria from the selected genus; and
(ii) at least one agent that inhibits growth and/or survival of non-replicating persister forms of bacteria from the selected genus.

20. The method of claim 19, further comprising one or more steps selected from the group consisting of:
(b) obtaining from the subject a biological sample comprising one or more morphological forms of bacteria from the selected genus;
(c) isolating at least one of the morphological forms of the bacteria;
(d) culturing the isolated bacteria; and
(e) assessing the susceptibility of the cultured bacteria to the at least one agent that inhibits the growth and/or survival of replicating forms of bacteria, the at least one agent that inhibits the growth and/or survival of non-replicating persister forms of bacteria, or both.
